(19) United States
(12) Patent Application Publication
(10) Pub. No.: US 2023/0059186 A1
(43) Pub. Date: Feb. 23, 2023

(54) METHODS AND COMPOSITIONS RELATED TO A HYBRID DNA REPAIR GLYCOSYLASE AND A THERMOSTABLE DNA LYASE

(71) Applicant: BOARD OF REGENTS, THE UNIVERSITY OF TEXAS SYSTEM, Austin, TX (US)

(72) Inventors: Lawrence Sowers, Galveston, TX (US); Mark Sowers, Galveston, TX (US); Chia Wei Hsu, Galveston, TX (US); Baljinnyam Tuvshintugs, Galveston, TX (US)

(21) Appl. No.: 17/748,871

(22) Filed: May 19, 2022

Related U.S. Application Data
(60) Provisional application No. 63/338,001, filed on May 3, 2022.
(60) Provisional application No. 63/226,140, filed on Jul. 27, 2021.

(51) Int. Cl. C12N 15/10; C12N 15/63; C12N 9/24

(52) U.S. Cl. CPC C12N 15/102; C12N 15/63; C12N 9/2497; C12Y 302/02029

(57) ABSTRACT

Certain embodiments are directed to compositions and methods for solving problems associated with measuring T:G mispairs, U:G mispairs and other 5-substituted uracil mispairs. Certain embodiments are directed to a hybrid enzyme that is capable of finding and cutting the T of the T:G mispair or other mispaired uracil analogs creating a method for their measurement. In certain embodiments the hybrid enzyme is a fusion of a human thymine DNA glycosylase (TDG) activator segment and a catalytic domain of an archaeal thermophilic thymine glycosylase (tTDG).

### RELATED APPLICATIONS

This application claims priority to U.S. Provisional Patent Application Ser. No. 63/226,140 filed Jul. 27, 2021 and 63/338,001 filed May 3, 2022, each of which is incorporated herein by reference in its entirety.

### STATEMENT REGARDING FEDERALLY FUNDED RESEARCH

This invention was made with government support under R01CA184097, R01CA228085, and F30CA225116 awarded by the National Institutes of Health (NIH). The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Cytosine to thymine transition mutations are the most abundant single-base changes observed in human cancer cells (1-5). These mutations are believed to arise from the hydrolytic deamination of cytosine and cytosine analogs (6-10) generating a mispaired intermediate with guanine (FIG. 1). The deaminated bases comprise an important class of DNA adducts; however, they cannot be measured by current approaches. Methods are therefore needed to measure the formation and persistence of the deaminated mispairs.

Several laboratories have developed sensitive and specific methods for measuring a wide array of DNA base adducts; however, such methods would require either enzymatic or acid hydrolysis prior to analysis (11-16). The mutagenic significance of the deaminated cytosine adducts is a consequence of residing in a base mispair with guanine, and DNA hydrolysis eliminates the base-pairing context. Further, PCR-based analytical methods would convert the mispaired intermediate to a G:C base pair and an A:T mutation, erasing the initial mispair context as well.

Other laboratories have used DNA repair glycosylases to selectively remove damaged bases from DNA for analysis by mass spectrometry-based methods (17-21). Uracil-DNA glycosylase (UDG) has been used to measure total uracil in DNA; however, UDG removes uracil from single-stranded DNA as well as U:A and U:G base pairs and therefore cannot distinguish a deaminated base pair (U:G) from a dUTP misincorporation event (U:A). On the other hand, Thymine DNA glycosylases (TDG) can remove uracil and thymine selectively from mispairs (U:G and T:G). However, the activity of the human thymine DNA glycosylase (hTDG) is very weak against T:G (22,23).

There remains a need for additional reagents and methods for measuring the formation and persistence of deaminated mispairs.

### SUMMARY

Embodiments are directed to compositions and methods for solving problems associated with measuring T:G mispairs, U:G mispairs and other 5-substituted uracil mispairs (xU:G) where xU can be but is not limited to 5-fluorouracil, 5-chlorouracil, 5-bromouracil, 5-iodouracil, 5-hydroxymethyluracil, 5-formyluracil and 5-carboxyuracil. Certain embodiments are directed to a hybrid enzyme that is capable of finding and cutting the T of the T:G mispairs and other analogs creating a method for their measurement.

In certain embodiments the hybrid enzyme is a fusion of a human thymine DNA glycosylase (TDG) segment and a catalytic domain of an archaeal thermophilic thymine glycosylase (tTDG). In certain aspects, the hybrid TDG (hyTDG) was generated by joining a 29 amino acid sequence segment shown to substantially increase the activity of hTDG to the catalytic core of tTDG.

Certain embodiments are directed to a hybrid glycosylase polypeptide comprising an amino terminal human TDG activator segment (activator segment) linked to a catalytic domain of a thermophile TDG (catalytic segment). In certain embodiments the activator segment and the catalytic segment are connected by a peptide bond, i.e., are a fusion protein. The polypeptides of the invention can include one or more polypeptide tags. Polypeptide tags include but are not limited to an immunoglobulin Fc polypeptide, an immunoglobulin mutein Fc polypeptide, a hemagglutinin peptide, a calmodulin binding polypeptide (or a domain or peptide thereof), a protein C-tag, a streptavidin binding peptide (or fragments thereof), a protein A fragment (e.g., an IgG-binding ZZ polypeptide), a Softag™ peptide, a polyhistidine tag (his tag, hex-histidine tag), FLAG® epitope tag (DYKDDDDK, SEQ ID NO:175), beta-galactosidase, alkaline phosphatase, GST, the XPRESS™ epitope tag (DLYDDDDK, SEQ ID NO:176; (Invitrogen Corp., Carlsbad, Calif.)), and the like. In certain aspects, the hybrid glycosylase polypeptide includes a polyhistidine tag. In certain aspects, the tag is an amino terminal tag.

In certain aspects, the amino terminal human activator segment has an amino acid sequence of SKKSGKSAKSKEKQEKITDTFKVKRKVDR (SEQ ID NO:2) or a variant thereof. A variant of the activator segment can have 1, 2, 3, 4, 5, 6, 7, 8, 9, or 10 amino acid substitutions while maintaining its function in activating the catalytic segment. One or more of the amino acid substitutions can be a conservative amino acid substitution. A variant of the activator segment can have a 1, 2, 3, 4, 5, 6, 7, 8, 9, or 10 amino acid deletions while maintaining its function in activating the catalytic segment. In certain aspects the deletion the activator segment can be a 1, 2, 3, 4, 5 consecutive amino acid terminal deletion. The terminal deletion can be an amino terminal or carboxy terminal deletion. A variant of the activator segment can have a 1, 2, 3, 4, 5, 6, 7, 8, 9, or 10 amino acid additions while still maintaining its function in activating the catalytic segment. The amino acid addition can be a terminal addition or an insertion of amino acid in the activator segment. In certain aspects, an addition to the activator segment can be a 1, 2, 3, 4, 5, 7, 8, 9, 10 or more consecutive amino acid terminal addition. The terminal addition can be an amino terminal or carboxy terminal deletion relative to the activator segment, for example the addition can be a carboxy terminal addition of amino acid relative to the activator segment which results in an insertion between the activator segment and the catalytic segment. In certain aspects, the addition is a tag, such as a hexa-histidine tag or similar segment. The variant of the amino terminal human activator segment can have one or more amino acid substitution(s), deletion(s), or addition(s).

A thermophile is an organism that thrives at relatively high temperatures, between 41 and 122° C. Many thermophiles are archaea, though they can also be bacteria. Archaea constitute a domain of single-celled organisms that lack cell nuclei and are therefore prokaryotes. In certain aspects, the thermophile TDG glycosylase (tTDG) is a Methanobacterium thermoautotrophicum tTDG also known as Methanobacterium thermoformicium (26-28). In certain aspects, the catalytic segment of a thermophile TDG has an amino acid sequence that is or is at least 60, 65, 70, 75, 80, 85, 90, 91, 92, 93, 94, 95, 96, 97, 98, 99, or 100% identical to 200, 201, 202, 203, 204, 205, 206, 207, 208, 209, 210, 211, 212, 213, 214, 215, 216, 217, 218, 219, 220, 221 consecutive amino acids from amino acid 1, 2, 3, 4, 5, 6, 7, 8, 9, or 10 of LDDATNKKRKVFVSTILTFWNTDRRDFPWRHTRDPYVIIITEILLRRTTAGHVKKIYDKF FVKYKCFEDILKTPKSEIAKDIKEIGLSNQRAEQLKELARVVINDYGGRVPRNRKAILDLP GVGKYTCAAVMCLAFGKKAAMVDANFVRVINRYFGGSYENLNYNHKALWELAETLV PGGKCRDFNLGLMDFSAIICAPRKPKCEKCGMSKLCSYYEKCST (SEQ ID NO:3) or a variant thereof. A variant of the catalytic segment can have 1, 2, 3, 4, 5, 6, 7, 8, 9, or 10 amino acid substitutions while maintaining its function as the catalytic segment. One or more of the amino acid substitutions can be a conservative amino acid substitution. A variant of the catalytic segment can have a 1, 2, 3, 4, 5, 6, 7, 8, 9, or 10 amino acid deletions while maintaining its function as the catalytic segment. In certain aspects, the deletion in the catalytic segment can be a 1, 2, 3, 4, 5 consecutive amino acid terminal deletion, relative to the catalytic segment. The terminal deletion can be an amino terminal or carboxy terminal deletion. A variant of the catalytic segment can have a 1, 2, 3, 4, 5, 6, 7, 8, 9, or 10 amino acid additions while still maintaining its function as the catalytic segment. In certain aspects, an addition to the catalytic segment can be a 1, 2, 3, 4, 5, 7, 8, 9, 10 or more consecutive amino acid terminal addition. The terminal addition can be an amino terminal or carboxy terminal deletion relative to the catalytic segment. The variant of the catalytic segment can have one or more amino acid substitution(s), deletion(s), or addition(s).

In certain aspects, the hybrid glycosylase polypeptide includes an amino acid segment that is or is at least 60, 65, 70, 75, 80, 85, 90, 91, 92, 93, 94, 95, 96, 97, 98, 99, or 100% identical to 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 consecutive amino acids from amino acid 1, 2, 3, 4, 5, 6, 7, 8, 9, 10, 11, 12, 13, 14, or 15 of SKKSGKSAKSKEKQEKITDTFKVKRKVDRLDDATNKKRKVFVSTILTFWNTDRRDFPW RHTRDPYVILITEILLRRTTAGHVKKIYDKFFVKYKCFEDILKTPKSEIAKDIKEIGLSNQR AEQLKELARVVINDYGGRVPRNRKAILDLPGVGKYTCAAVMCLAFGKKAAMVDANFV RVINRYFGGSYENLNYNHKALWELAETLVPGGKCRDFNLGLMDFSAIICAPRKPKCEKC GMSKLCSYYEKCST (SEQ ID NO:1) or a variant thereof. A variant of the polypeptide can have 1, 2, 3, 4, 5, 6, 7, 8, 9, or 10 amino acid substitutions while maintaining its function. One or more of the amino acid substitutions can be a conservative amino acid substitution. A variant of the polypeptide can have a 1, 2, 3, 4, 5, 6, 7, 8, 9, or 10 amino acid deletions while maintaining its function. In certain aspects, the deletion in the polypeptide can be a 1, 2, 3, 4, 5 consecutive amino acid terminal deletion. The terminal deletion can be an amino terminal or carboxy terminal deletion. A variant of the polypeptide can have a 1, 2, 3, 4, 5, 6, 7, 8, 9, or 10 amino acid additions while still maintaining its function. In certain aspects, an addition to the polypeptide can be a 1, 2, 3, 4, 5, 7, 8, 9, 10 or more consecutive amino acid terminal addition. The variant of the polypeptide can have one or more amino acid substitution(s), deletion(s), or addition(s). In certain aspects, the polypeptide has an amino acid sequence that is or is at least 90, 91, 92, 93, 94, 95, 96, 97, 98, 99, or 100% identical to the amino acid sequence of SEQ ID NO:1.

Other embodiments are directed to methods of evaluating the activity of a hybrid glycosylase polypeptide described herein comprising: (i) incubating a hybrid glycosylase polypeptide as described herein with a nucleic acid comprising a fluorophore/quencher pair generating an abasic site; (ii) cleaving the abasic site by contact with a cleavage reagent; (iii) measuring fluorescence intensity, which is indicative of mispaired pyrimidine content, (iv) measuring hybrid glycosylase activity using a gel-based assay with fluorescence or ³²P-labeled oligonucleotide substrates.

Certain embodiments are directed to a nucleic acid or expression cassette encoding a hybrid glycosylase polypeptide as described herein.

Certain embodiments are directed to a cell expressing a hybrid glycosylase polypeptide as described herein. The cell can be a prokaryotic or eukaryotic cell. In certain aspects the cell is a bacterial cell. In certain aspects the polypeptide is isolated from a hybrid glycosylase polypeptide expressing cell.

Certain embodiments are directed to a kit for expressing or using a hybrid glycosylase polypeptide described herein.

Certain embodiments are directed to methods for measuring pyrimidines comprising: (i) incubating a hybrid glycosylase polypeptide as described herein with a nucleic acid producing free bases; (ii) derivatizing the free bases; (iii) isolating the derivatized free bases; and (iv) analyzing the derivatized free bases by GC-MS/MS or size fractionation.

Themostable DNA Lyase—Certain embodiments are directed to a hybrid thymine DNA lyase (hyTDG-lyase). A tyrosine to lysine substitution at position 163 of SEQ ID NO:28

3 (referred to herein as Y163K) the hybrid thymine DNA glycosylase (hyTDG) was constructed forming the hyTDG-lyase. The mutant protein had an apparent molecular weight of 26.5 kDa (FIG. 27). An example of an amino acid sequence of the hyTDG-lyase is shown in FIG. 20A (SEQ ID NO:186, noting that amino acids 1 to 8 comprise an amino terminal histidine tag that may or may not be present).

In certain embodiments the hybrid enzyme is a fusion of a human thymine DNA glycosylase (TDG) segment and a catalytic domain of an archaeal thermophilic thymine glycosylase (tTDG) having the Y163K substitution producing a hybrid thymine DNA lyase (hyTDG-lyase).

Certain embodiments are directed to a hyTDG-lyase polypeptide comprising an amino terminal human TDG activator segment (activator segment) linked to a variant catalytic domain of a thermophile TDG (catalytic segment). In certain embodiments the activator segment and the variant catalytic segment are connected by a peptide bond, i.e., are a fusion protein. The polypeptides of the invention can include one or more polypeptide tags. Polypeptide tags include but are not limited to an immunoglobulin Fc polypeptide, an immunoglobulin mutein Fc polypeptide, a hemagglutinin peptide, a calmodulin binding polypeptide (or a domain or peptide thereof), a protein C-tag, a streptavidin binding peptide (or fragments thereof), a protein A fragment (e.g., an IgG-binding ZZ polypeptide), a Softag™ peptide, a polyhistidine tag (his tag, hex-histidine tag), FLAG® epitope tag (DYKDDDDK, SEQ ID NO:175), beta-galactosidase, alkaline phosphatase, GST, the XPRESS™ epitope tag (DLYDDDDK, SEQ ID NO:176; (Invitrogen Corp., Carlsbad, Calif.)), and the like. In certain aspects, the hyTDG-lyase polypeptide includes a polyhistidine tag (e.g., amino acids 1 to 8 of SEQ ID NO:186). In certain aspects, the tag is an amino terminal tag.

In certain aspects, the amino terminal human activator segment has an amino acid sequence of SKKSGKSAKSKEKQEKITDTFKVKRKVDR (SEQ ID NO:2) or a variant thereof. The variant activator segment can have 1, 2, 3, 4, 5, 6, 7, 8, 9, or 10 amino acid substitutions while maintaining its function in activating the catalytic segment and having a Y163K substitution. One or more of the amino acid substitutions in the activator segment can be a conservative amino acid substitution. A variant of the activator segment can have a 1, 2, 3, 4, 5, 6, 7, 8, 9, or 10 amino acid deletions while maintaining its function in activating the catalytic segment. In certain aspects the deletion the activator segment can be a 1, 2, 3, 4, 5 consecutive amino acid terminal deletion. The terminal deletion can be an amino terminal or carboxy terminal deletion. A variant of the activator segment can have a 1, 2, 3, 4, 5, 6, 7, 8, 9, or 10 amino acid additions while still maintaining its function in activating the catalytic segment. The amino acid addition can be a terminal addition or an insertion of amino acid in the activator segment. In certain aspects, an addition to the activator segment can be a 1, 2, 3, 4, 5, 7, 8, 9, 10 or more consecutive amino acid terminal addition. The terminal addition can be an amino terminal or carboxy terminal deletion relative to the activator segment, for example the addition can be a carboxy terminal addition of amino acid relative to the activator segment which results in an insertion between the activator segment and the catalytic segment. In certain aspects, the addition is a tag, such as a hexa-histidine tag or similar segment. The variant of the amino terminal human activator segment can have one or more amino acid substitution(s), deletion(s), or addition(s).

In certain aspects, the thermophile TDG glycosylase (tTDG) is a Methanobacterium thermoautotrophicum tTDG also known as Methanobacterium thermoformicium (26-28). In certain aspects, the catalytic segment of a thermophile TDG has an amino acid sequence that is or is at least 60, 65, 70, 75, 80, 85, 90, 91, 92, 93, 94, 95, 96, 97, 98, or 99% identical to 200, 201, 202, 203, 204, 205, 206, 207, 208, 209, 210, 211, 212, 213, 214, 215, 216, 217, 218, 219, 220, 221 consecutive amino acids from amino acid 1, 2, 3, 4, 5, 6, 7, 8, 9, or 10 of LDDATNKKRKVFVSTILTFWNTDRRDFPWRHTRDPYVIIITEILLRRTTAGHVKKIYDKF FVKYKCFEDILKTPKSEIAKDIKEIGLSNQRAEQLKELARVVINDYGGRVPRNRKAILDLP GVGKKTCAAVMCLAFGKKAAMVDANFVRVINRYFGGSYENLNYNHKALWELAETLV PGGKCRDFNLGLMDFSAIICAPRKPKCEKCGMSKLCSYYEKCST (SEQ ID NO:187) or a variant thereof, while maintaining the Y163K substitution which corresponds to a Y126K substitution in SEQ ID NO:187. A variant of the catalytic segment can have 1, 2, 3, 4, 5, 6, 7, 8, 9, or 10 amino acid substitutions while maintaining its function as the catalytic segment-maintaining the Y163K/Y126K substitution. One or more of the amino acid substitutions can be a conservative amino acid substitution. A variant of the catalytic segment can have a 1, 2, 3, 4, 5, 6, 7, 8, 9, or 10 amino acid deletions while maintaining its function as the catalytic segment. In certain aspects, the deletion in the catalytic segment can be a 1, 2, 3, 4, 5 consecutive amino acid terminal deletion, relative to the catalytic segment. The terminal deletion can be an amino terminal or carboxy terminal deletion. A variant of the catalytic segment can have a 1, 2, 3, 4, 5, 6, 7, 8, 9, or 10 amino acid additions while still maintaining its function as the catalytic segment. In certain aspects, an addition to the catalytic segment can be a 1, 2, 3, 4, 5, 7, 8, 9, 10 or more consecutive amino acid terminal addition. The terminal addition can be an amino terminal or carboxy terminal deletion relative to the catalytic segment. The variant of the catalytic segment can have one or more amino acid substitution(s), deletion(s), or addition(s).

In certain aspects, hyTDG-lyase polypeptide includes an amino acid segment that is or is at least 60, 65, 70, 75, 80, 85, 90, 91, 92, 93, 94, 95, 96, 97, 98, 99, or 100% identical to 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 consecutive amino acids from amino acid 1, 2, 3, 4, 5, 6, 7, 8, 9, 10, 11, 12, 13, 14, or 15 of SKKSGKSAKSKEKQEKITDTFKVKRKVDRLDDATNKKRKVFVSTILTFWNTDRRDFPW RHTRDPYVILITEILLRRTTAGHVKKIYDKFFVKYKCFEDILKTPKSEIAKDIKEIGLSNQR AEQLKELARVVINDYGGRVPRNRKAILDLPGVGKKTCAAVMCLAFGKKAAMVDANFV RVINRYFGGSYENLNYNHKALWELAETLVPGGKCRDFNLGLMDFSAIICAPRKPKCEKC GMSKLCSYYEKCST (SEQ ID NO:189) or a variant thereof while maintaining the Y163K substitution, which corresponds to Y155K substitution in SEQ ID NO:189. A variant of the polypeptide can have 1, 2, 3, 4, 5, 6, 7, 8, 9, or 10 amino acid substitutions while maintaining its function. One or more of the amino acid substitutions can be a conservative amino acid substitution. A variant of the polypeptide can have a 1, 2, 3, 4, 5, 6, 7, 8, 9, or 10 amino acid deletions while maintaining its function. In certain aspects, the deletion in the polypeptide can be a 1, 2, 3, 4, 5 consecutive amino acid terminal deletion. The terminal deletion can be an amino terminal or carboxy terminal deletion. A variant of the polypeptide can have a 1, 2, 3, 4, 5, 6, 7, 8, 9, or 10 amino acid additions while still maintaining its function. In certain aspects, an addition to the polypeptide can be a 1, 2, 3, 4, 5, 7, 8, 9, 10 or more consecutive amino acid terminal addition. The variant of the polypeptide can have one or more amino acid substitution(s), deletion(s), or addition(s). In certain aspects, the polypeptide has an amino acid sequence that is or is at least 90, 91, 92, 93, 94, 95, 96, 97, 98, 99, or 100% identical to the amino acid sequence of SEQ ID NO:186.

Certain embodiments are directed to a nucleic acid or expression cassette encoding hyTDG-lyase polypeptide as described herein.

Certain embodiments are directed to a cell expressing hyTDG-lyase polypeptide as described herein. The cell can be a prokaryotic or eukaryotic cell. In certain aspects the cell is a bacterial cell.

Certain embodiments are directed to a kit for expressing or using hyTDG-lyase polypeptide described herein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Pathway for mutations induced by deamination of cytosine and analogs. Deaminated intermediates can be converted to mutation by DNA replication or repair synthesis. Mispaired intermediates can also be repaired by excision repair pathways.

FIG. 2. Amino acid sequence of hyTDG (SEQ ID NO:1). The 29 amino acid peptide derived from hTDG is shown in underline and significant peptides observed by mass spectrometry are shown in bold.

FIG. 3. Mass spectrum of peptide KVDR/LDDATNK (SEQ ID NO:39) which is junction between hTDG and tTDG sequences. The sequence of the peptide is determined from examination of the b or y ion fragments as indicated in the figure.

FIG. 4. Comparison of UNG, hyTDG and hTDG activity using a gel cleavage assay. Single-stranded oligonucleotides or duplexes containing indicated base pairs and a 5′-fluorescein tag (2.5 pmol) were incubated with hyTDG for 1 h at 65° C. Sodium hydroxide was then added to hydrolyze the phosphate backbone of oligonucleotides containing an abasic site. UDG cleaves all uracil-containing oligonucleotides, but not T. The hyTDG cleaves only mispaired U and T and other 5-substituted uracil analogs. The human TDG cleaves U mispaired with G but little T mispaired with G.

FIGS. 5A and 5B. Analysis of glycosylase activity of hyTDG on oligonucleotides using a real-time fluorescence assay. (A) 25 pmol duplexes with 5′-FAM and 3′-BHQ1 were incubated with hyTDG. Fluorescence was monitored in a Roche 480 qPCR instrument. (B) as with (A) but with addition of 20 μg calf thymus DNA. Neither U:A or T:A-contining sequences were cleaved by hyTDG.

FIG. 6. Workflow for measuring bases released by hyTDG using mass spectrometry. Oligonucleotides or DNA are incubated with hyTDG or UDG in the presence of one or more stable-isotope standards. Following incubation, free bases are separated from oligonucleotides or DNA using a spin column. Isolated bases are silylated and analyzed by GC-MS/MS. Pyrimidines released by the glycosylase are quantified by comparing the integrated peak area of the unenriched pyrimidine with a corresponding stable isotope enriched standard.

FIGS. 7A and 7B. Cleavage of a mixture of oligonucleotides containing T:G and U:G mispairs by hyTDG followed simultaneously by gel electrophoresis and GC-MS/MS. Mixtures of 50-FAM-labeled oligonucleotides containing U:G (8.3 pmol) or T:G (16.7 pmol) were incubated with 250 pmol hyTDG and isotope-enriched standards (U+3, T+4) in a total volume of 25 μl at 65° C. At selected time intervals, 5 μl was used for gel electrophoresis, and the remaining 20 μl was used for the measurement of released bases. Released bases were separated by spin filtration, derivatized, and analyzed by GC-MS/MS. Gel analysis indicated predominant cleavage of U:G and T:G oligonucleotides by hyTDG by 60 min (panel A). The oligonucleotide mixture was also incubated with UDG at 37° C. (1 unit, 0.6 pmol), which cleaved the U:G but not T:G oligonucleotide (panel A, far right). Base release was measured by GC-MS/MS as shown in panel B. Each time point was analyzed three times. At 2 h, 6.42±0.49 pmol U and 13.33±0.34 pmol were released, representing nearly complete release of U:G and T:G in the sample. The amount of U released by UDG at 2 h was 7.58±0.2 pmol. FAM, 6-carboxyfluorescein; hyTDG, hybrid thymine DNA glycosylase.

FIGS. 8A and 8B. Analysis of the release of U and mispaired T from calf thymus DNA by UDG and hyTDG. Approximately 400 μg of EcoRI-digested calf thymus DNA was incubated with UDG (10 units, 6.2 pmol, 37° C.) or hyTDG (295 pmol, 65° C.) for 90 min. Released bases were isolated by spin filtration, derivatized, and analyzed by GC-EI-MS/MS (panel A) or GC-NCI-MS (panel B). Data presented above represents observed amounts minus background from three independent experiments. In panel A, total uracil (single stranded, U:A and U:G) released by UDG was 9.39±0.29 pg/μg DNA. The amount of uracil from U:G released by hyTDG was 1.30±0.29 pg/μg, and the amount of T from T:G released was 5.58±0.42 pg/μg. These amounts correspond to one deaminated U:G mispair per 4.48×104 C:G base pairs and one deaminated T:G mispair per 6.71×102 5-mC:G base pairs. In panel B, total uracil (single strand, U:A and U:G) released by UDG was 8.46±0.63 pg/μg DNA. The amount of uracil from U:G released by hyTDG was 0.54±0.13 pg/μg, and the amount of T from T:G released was 4.14±0.21 pg/μg. These amounts correspond to one deaminated U:G mispair per 1.08×105 C:G base pairs and one deaminated T:G mispair per 9.09×102 5-mC:G base pairs. Most of the U is in U:A base pairs or single-stranded DNA (86% panel A, 94% panel B). The amount of T in T:G mispairs exceeds the amount of U in U:G mispairs by a factor of 4.3 (panel A) to 7.7 (panel B). EI, electron ionization; hyTDG, hybrid thymine DNA glycosylase; NCI, negative chemical ionization; UDG, uracil-DNA glycosylase.

FIG. 9. DNA sequence. One example of a DNA Sequence of hyTDG vector.

FIG. 10. Protein gel for purified hyTDG. Lane 1, protein MW standards. Lane 2, purified hyTDG. Lane 3, albumin.

FIG. 11. Mass spectrum of peptide SKEKQEKITDTFK (SEQ ID NO:21). Derived from the 29 amino acids peptide from the N-terminal region of hTDG.

FIG. 12. Mass spectrum of peptide DPYVILITEILLR (SEQ ID NO:8). The peptide DPYVILITEILLRR (SEQ ID NO:19)(amino acids 69-74) contains the “R” base flipper for this class of glycosylase

FIG. 13. Mass spectrum of peptide KAILDLPGVGK (SEQ ID NO:26). This peptide contains the LPGVGKY (SEQ ID NO:172) helix-hairpin-helix (HhH). The HhH motif consists of two α-helices flanking a β-hairpin with the conserved LPGVGX(K/S) (SEQ ID NO:173) which binds the DNA backbone non-specifically. The HhH motif places the thermophile TDG (tTDG) in the same class with the Mut Y and Endo III glycosylases.

FIG. 14. Mass spectrum of peptide KAAMVDANFVR (SEQ ID NO:24). This peptide contains a conserved aspartic acid residue (bold) common to Mut Y, Endo III and tTDG glycosylases which is catalytic and interacts with the C1′ position of the target 2′-deoxyribose.

FIG. 15. Mass spectrum of peptide DFNLGLMDFSAIICAPR (SEQ ID NO:174). This peptide contains the first cysteine residue of the iron-sulfur cluster common to Mut Y, EndoIII and several other glycosylases.

FIG. 16. Sequence of oligonucleotides used in cleavage assays. The molar extinction coefficients (M⁻¹ cm⁻¹) used to calculate the concentrations of the oligonucleotide solutions were as follows: FAM-T (5′-6FAM sequence containing red T, 207,943), FAM-u (5′-FAM sequence containing red U, 206,526), BHQ-G (3′-BHQ1 sequence containing bold G, 180,472) and BHQ-A (3′-BHQ1 sequence with bold A, 183,654).

FIG. 17. Added uracil does not inhibit hyTDG cleavage of duplexes containing T:G or U:G mispairs. The reaction contained 5 pmol of duplex plus hyTDG. Uracil was added up to a final concentration of 50 pmol.

FIG. 18. MS/MS. Separation of the tert-butyldimethylsilyl (TBDMS) derivatives of uracil and thymine by GC and detection by MS/MS.

FIG. 19. Schematic of lyase activity in short-patch base excision repair (BER). Following glycosylase removal of a damaged or mispaired base by a DNA glycosylase, the DNA phosphodiester backbone can be cleaved by nonenzymatic hydrolysis (β or δ-elimination) or by a DNA lyase. DNA lyases can cleave on the 5′-side of the abasic site (i.e., APE 1) or on the 3′-side (i.e., endo III). The DNA ends generated by lyases at the repair gap are a 3′-hydroxyl and a 5′-phosphate. DNA polymerase can extend from the 3′-hydroxyl in the presence of a complementary dNTP, and the repair cycle is completed by a DNA ligase. MALDI-TOF-MS data shows that the hyTDG lyase cleaves on the 3′-side of the abasic site, generating a 5′-deoxyribose phosphate terminus. However, in the presence of β-mercaptoethanol (β-ME), an adduct is formed with an increased mass of 60 amu. One of the possible isomers of this adduct is shown.

FIG. 20A-20B. hyTDG-lyase amino acid sequence and confirmation of amino acid sequence. (A) Amino acid sequence of hyTDG-lyase (SEQ ID NO:186). The protein has a 6×his tag on the amino terminal. The 29 amino acid sequence from human thymine DNA glycosylase (hTDG) is SKKSGKSAKSKEKQEKITDTFKVKRKVDR (SEQ ID NO:2). A tyrosine at position 163 is substituted by a lysine (Y163K). (B) Mass spectrum of the NRKAILDLPGVGKK (SEQ ID NO:188) peptide containing the Y163K substitution obtained by nLC-MS/MS. The fragmentation pattern confirms the predicted sequence.

FIG. 21A-21B. MALDI-TOF-MS of the fragments resulting from cleavage of an abasic site-containing oligonucleotide by hyTDG-lyase. An 18 base with a 5′-FAM label and a U:G mispair was incubated with UDG for 1 h, followed by incubation with hyTDG-lyase. The resulting fragments were examined by MALDI-TOF-MS. (A) The 5′-fragment contains a 5′-FAM label with a measured m/z of 2601.2450. The observed mass is consistent with the formation of an adduct with β-mercaptoethanol (theoretical m/z 2601.48). (B) The corresponding 3′-fragment has a phosphate on its 5′-end and a measured m/z of 3446.3311 (theoretical m/z 3446.58).

FIG. 22A-22E. hyTDG-lyase is thermostable. A 5′-FAM labelled 18 base oligonucleotides containing a T:G mispair (2.5 pmol) was incubated with hyTDG glycosylase (16.8 pmol, 65° C., 1 h) in TDG buffer (10 mM K₂HIPO₄, 30 mM NaCl, 40 mM KCl, pH 7.7) followed by either hyTDG-lyase (16.8 pmol), APE 1 (5 units), Fpg (5 units), no lyase control or intact oligonucleotide (oligo only) at the indicated temperatures (° C.) for 1 h. Samples were then mixed with an equal volume of formamide and resolved on a 20% denaturing polyacrylamide gel. Images were visualized using Storm gel imager. (A) hyTDG-lyase cleaves oligonucleotides at an abasic site generated by hyTDG at all temperatures tested. (B) APE 1 cleaves oligonucleotide at an abasic site generated by hyTDG from 25-45° C., and it is inactive at higher temperatures (55-95° C.). Spontaneous δ-elimination occurs at the AP site at higher temperatures (55-95° C.). (C) Fpg cleaves oligonucleotides at an abasic site generated by hyTDG at 25-55° C., and it is inactive at higher temperatures (65-95° C.). Increased temperatures caused δ-elimination at AP site resulting in a slightly slower migrating band. (D) An abasic site generated by hyTDG (no lyase), undergoes spontaneous δ-elimination with increasing temperature. (E) An intact oligonucleotide with no abasic site is stable to hydrolysis under the conditions employed in this experiment.

FIG. 23A-23B. hyTDG-lyase is active in multiple buffers. (A) A 5′-FAM labelled 18 base U:G oligonucleotides (2.5 pmol) was incubated with UDG (2.5 units) followed by NaOH (160 μM, 96° C., 10 min), hyTDG-lyase (16.8 pmol) or APE 1 (5 units) for 1 h in indicated buffers. Buffer 1: TDG buffer (10 mM K₂HIPO₄, 30 mM NaCl, 40 mM KCl, pH 7.7); Buffer 2: UDG buffer (20 mM Tris-HCl, 1 mM DTT, 1 mM EDTA, pH 8.0); Buffer 3: NEBuffer™ 1 buffer (1 mM DTT, 10 mM Bis Tris-Propane HCl, 10 mM MgCl₂, pH 7.0). The hyTDG-lyase is active in all three buffers whereas APE 1 is active in buffers 1 and 3. (B) A 5′-FAM labelled 18 base 5foC:G-containing oligonucleotides (2.5 pmol) was incubated with hTDG (31 pmol, 37° C., 1 h) to remove the 5foC and generate an abasic site. The phosphodiester backbones of the abasic-site containing oligonucleotide was then cleaved by incubation with NaOH, hTDG-lyase or APE 1 in buffer 1 or buffer 2. Oligonucleotide fragments were resolved by gel electrophoresis and imaged with a Storm imager. NaOH induces hydrolytic degradation of 5foC, as indicated by cleavage in NaOH, even in the absence of a glycosylase. The hyTDG-lyase cleaves oligonucleotides containing an abasic site, generated by hTDG excision of 5foC, in both buffer 1 and buffer 2. APE 1 cleaves oligonucleotides containing an abasic site generated by hTDG excision of 5foC completely in buffer 1 but inefficiently in buffer 2.

FIG. 24A-24B. Cleavage by hyTDG-lyase opposite G is faster than opposite A, T, C or in a single-stranded oligonucleotide. (A) 5′-FAM labeled oligonucleotides containing U in a single-stranded oligonucleotide or in duplexes containing U paired with G, A, T and C were incubated with UDG (2.5 pmol) in UDG buffer (20 mM Tris-HCl, 1 mM DTT, 1 mM EDTA, pH 8.0, at 37° C. for 1 h to generate abasic sites. hyTDG-lyase was then added, and the cleavage of the abasic-site containing oligonucleotides was measured at 65° C. at 1, 2 and 4 h. The oligonucleotides containing an abasic site paired opposite G are completely cleaved by 1 h. Single-stranded oligonucleotides as well as duplexes containing abasic sites opposite A, T and C are cleaved more slowly. (B) The cleavage of abasic-site containing oligonucleotides was also monitored using a real-time fluorescence assay. Oligonucleotide duplexes with a U:G or U:A and containing a 5′-FAM label in the upper strand and a 3′-BHQ quencher in the complementary strand were incubated with UDG for 1 h at 37° C. (UDG) to generate an abasic site. The hyTDG-lyase was then added and fluorescence was measured at 65° C. as a function of time in a qPCR instrument. Three independent experiments were performed and the data for each is shown in the figure. The equation for the solid lines in each figure is Y=A(1−e^{-kt}) where Y is the normalized fluorescence, A is maximum percent cleaved, k is the rate constant (min-1), and t is time in min. The average values of A and k for an abasic site opposite G (AP:G) were 98.8±0.5 and 0.0569±0.011 min⁻¹, and for AP:A were 106.8±3.10 and 0.0123±0.002 min⁻¹. In accord with the data in FIG. 6A, hyTDG-lyase cleaves abasic sites opposite G at twice the rate as when opposite A.

FIG. 25A-25B. hyTDG glycosylase and hyTDG-lyase can compete with one another. (A) 5′-FAM labelled 18 base U:G oligonucleotides (2.5 pmol) were incubated with hyTDG (16.8 pmol) in TDG buffer (10 mM K₂HIPO₄, 30 mM NaCl, 40 mM KCl, pH 7.7) at 65° C. plus increasing amounts of hyTDG-lyase. Optimum cleavage is observed with 8.4 pmol hyTDG-lyase. These data show that optimal oligonucleotide cleavage is obtained with a 2:1 ratio of hyTDG-lyase to hyTDG glycosylase, and that increasing the amount of hyTDG-lyase can diminish overall cleavage due to apparent competitive binding of hyTDG and hyTDG-lyase for the U:G mispair. (B) The experiment shown was a repeated with the addition of NaOH following coincubation with hyTDG and hyTDG-lyase. The NaOH was added to reveal the total amount of abasic sites present. When the amount of the hyTDG-lyase is twice that of the hyTDG glycosylase (far right lane) overall cleavage is diminished. These data indicate that the hyTDG-lyase could bind to the U:G mispair, preventing generation of an alkaline-labile abasic site. When the lyase to glycosylase ratio is less than 2, the hyTDG glycosylase can remain bound to its product, blocking cleavage by hyTDG-lyase.

FIG. 26. Oligonucleotides cleaved by hyTDG-lyase cannot be extended by DNA pol β during short-patch base excision repair. A 5′-FAM labelled 79-base oligonucleotide (2.5 pmol, lane 1) was incubated with UDG (2.5 units, 37° C., 1 h) in CutSmart™ buffer (50 mM potassium acetate, 20 mM tris-acetate, 10 mM magnesium acetate, 100 μg/ml BSA, pH 7.9) and then cleaved by APE 1 (5 units, lane 2) or hyTDG-lyase (26.9 pmol, lane 4) at 37° C. for 30 min. Repair following incubation with APE 1 was completed by addition of polβ (6.2 pmol), dCTP (20 μM) and ligase (5 units, lane 3). Repair was incomplete following incubation with hyTDG-lyase (lane 5). However, addition of APE 1 (5 units), pol b, dCTP and ligase following hyTDG-lyase incubation allowed the completion of repair (lane 6).

FIG. 27A-27B. Purified hyTDG-lyase. (A) N-terminal 6×His tagged hyTDG-lyase protein (predicted MW: 29.7 kDa) was purified from BL21 (DE3) E. coli cells using HisPur Ni-NTA Resin. Two-micrograms of purified hyTDG-lyase was separated in 12% Tris-glycine PAGE and stained with Coomassie Brilliant Blue. The hyTDG-lyase migrated at approximately 26.5 kDa (lane: 2) relative to the protein MW ladder (Precision Plus Protein Standards, Biorad #161-0374) (lane: 1), and BSA (2 μg) (lane: 3). (B) The original, uncropped gel.

FIG. 28A-28B. MALDI-Tof mass spectrum of endo III β-elimination oligonucleotide cleavage products. (A) Mass spectrum of the 5′-6carboxyfluorescein (FAM) containing end of an 18-base oligonucleotide with a U:G mispair incubated with UDG (1 unit) and endo III (10 units) simultaneously for 2 h at 37° C. The oligonucleotide was cleaved leaving a 5′-FAM base with a 3′-OH deoxyribose phosphate fragment that undergoes spontaneous hydration with an observed m/z of 2541.5757 Da (theoretical m/z 2541.48 Da). (B) The 11-base oligonucleotide from the 3′-end of the original oligonucleotide generated by endo III cleavage has a 5′-phosphate end as indicate by the observed m/z of 3446.7311 (theoretical m/z 3446.58 Da).

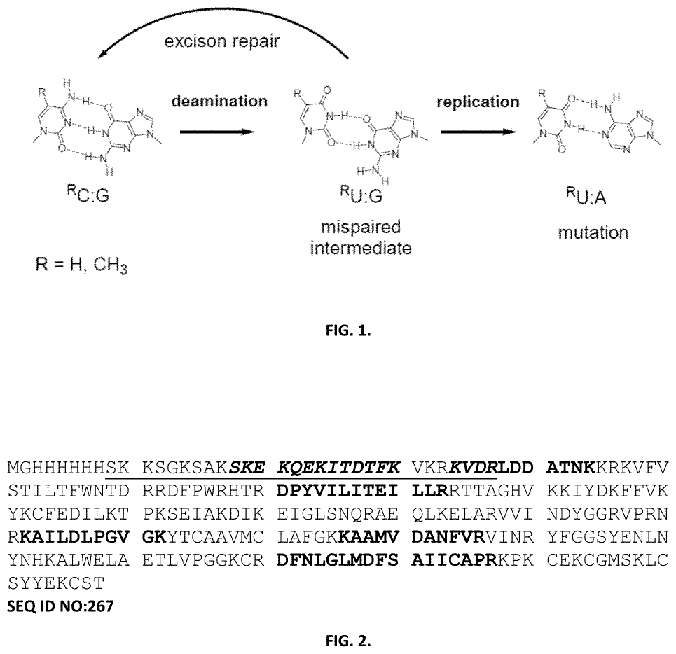

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

Currently, adequate methods are lacking for measuring deaminated intermediates. To address the lack of methods for measuring the deaminated intermediates, a hybrid glycosylase (hyTDG) has been constructed that cleaves uracil, thymine and other mispaired uracil analogs, key deamination products, selectively from mispairs. The hybrid enzyme can contain a 29 amino acid peptide from the human TDG or a variant thereof, shown to substantially increase the glycosylase activity of hTDG, human TDG activator segment (25). The human TDG activator segment can be linked or fused to the catalytic domain of a thermophile TDG. The rationale for linking the human peptide is that hTDG and other enzymes with thymine glycosylase activity are not robust, and that addition of the human sequence facilitates the overall glycosylase activity in the hybrid enzyme. The 29 amino acid N-terminal peptide of hTDG (residues 82-110) is unstructured and positively charged which may promote nonspecific interactions with the DNA phosphate backbone to promote lesion searching.

In contrast to human TDG (hTDG) which cleaves U:G>>T:G, the hybrid enzyme has strong activity against both U:G and T:G mispairs, fulfilling the needed activity for improving assays. A method has been developed to isolate and analyze bases released by glycosylases for subsequent analysis by mass spectrometry-based methods.

Uracil can occur in DNA by two distinct mechanisms (36-39). The deamination of cytosine in a duplex would generate a U:G mispair. Alternatively, dUMP could be misincorporated by DNA polymerase into an U:A base pair base pair during DNA replication. The amount of uracil in DNA from cytosine deamination (U:G) would increase with time and with UDG deficiency. Uracil misincorporation can occur during DNA replication into U:A base pairs as polymerases show little discrimination against dUTP. Uracil in DNA from misincorporation of dUMP would increase from defects in one-carbon metabolism and deficiencies in UDG or dUTPase activity.

Previous methods to measure uracil in DNA have relied upon UDG release or hydrolysis prior to analysis. Both methods measure total uracil. The biological significance of uracil in DNA depends upon the base pairing context. Uracil in U:A base pairs reflects metabolic disturbances and if unrepaired could interfere with DNA-protein interactions (40-42) whereas uracil in a U:G mispair is pro-mutagenic. Using the approach described herein, the distribution of uracil between U:A and U:G base pairs in DNA, for proof of concept calf thymus DNA, can be determined. Approximately 90% of the uracil in calf thymus DNA was found in U:A base pairs and therefore arose from dUMP misincorporation.

As with uracil, thymine could occur in a T:G base pair by deamination of 5mC or by the misincorporation of T opposite G during DNA replication. In human cancer cells, C to T mutations occur with high frequency at CpG dinucleotides (43-45). In eucaryotic DNA, cytosine methylation occurs predominantly at CpG dinucleotides. In addition, most CpG dinucleotides are methylated in most tissues (46-48). While polymerase misincorporation could generate a T:G mispair, available data suggests that polymerase misincorporation or extension is not strongly sequence-dependent (49,40). While 5mC deaminates slightly faster than cytosine (51,52), the repair of T:G mispairs in eucaryotic cells is lower than U:G mispairs by orders of magnitude. Therefore, the predominance of T:G mispairs in DNA likely arose from the deamination of 5mC. Using the methods described herein the inventors have measured the level of T:G base pairs in DNA. The inventors measured 965+/−54 fmol of T:G mispairs per μg of DNA. The level of T:G mispairs exceeds that of U:G mispairs by a factor of approximately 27 fmol, consistent with the slow repair of T:G mispairs in eucaryotic cells (53). The T:G mispair is a persistent DNA lesion, and the methods described herein could allow measurement of the rates of formation, repair and conversion to a mutation in human cells.

Endogenous DNA damage, including deamination and oxidation, is an important source of mutation in human cells, and it can generate apparent “noise” in next generation DNA sequencing studies. Recently, several groups have sought to reduce damaged-related noise by incubating DNA with a cocktail of DNA repairs enzymes prior to sequencing (54-60). A limitation of current approaches is that available repair enzymes do not efficiently act on the T:G mispair, which in described studies of calf thymus DNA is the most abundant aberrant base pair of the three examined. The hybrid TDG (hyTDG) described here should prove valuable in such assays.

### I. POLYPEPTIDE COMPOSITIONS

Certain embodiments are directed to a hybrid glycosylase polypeptide or a hyTDG-lyase comprising an amino terminal human TDG activator segment (activator segment) linked to a catalytic domain of a thermophile TDG (catalytic segment).

In certain embodiments, the polypeptide is a fusion polypeptide where the activator segment is linked at the N- or C-terminus to a catalytic segment forming a hybrid glycosylase polypeptide or a hyTDG-lyase. In other embodiments, the polypeptide comprises a linker interposed between the activator segment and the catalytic segment.

Furthermore, the polypeptides set forth herein may comprise a sequence of any number of additional amino acid residues at either the N-terminus or C-terminus of the amino acid sequence. For example, there may be an amino acid sequence of about 3 to about 100 or more amino acid residues at either the N-terminus, the C-terminus, or both the N-terminus and C-terminus of the polypeptide.

The polypeptide may include the addition of an antibody epitope or other tag, to facilitate identification, targeting, and/or purification of the polypeptide. The use of 6×His and GST (glutathione S transferase) as tags is well known. Inclusion of a cleavage site at or near the fusion junction will facilitate removal of the extraneous polypeptide after purification.

Polypeptides may possess deletions and/or substitutions of amino acids. Sequences with amino acid substitutions are contemplated, as are sequences with a deletion, and sequences with a deletion and a substitution. In some embodiments, these polypeptides may further include insertions or added amino acids.

Substitutional or replacement variants typically contain the exchange of one amino acid for another at one or more sites within the protein and may be designed to modulate one or more properties of the polypeptide, particularly to increase its efficacy or specificity. Substitutions of this kind may or may not be conservative substitutions. Conservative substitution is when one amino acid is replaced with one of similar shape and charge. Conservative substitutions are well known in the art and include, for example, the changes of alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Changes other than those discussed above are generally considered not to be conservative substitutions. It is specifically contemplated that one or more of the conservative substitutions above may be included. In some embodiments, such substitutions are specifically excluded. Furthermore, in additional embodiments, substitutions that are not conservative are employed in variants. In addition to a deletion or substitution, the polypeptides may possess an insertion of one or more residues. The hybrid glycosylase sequence can form the appropriate structure and conformation for its enzymatic function.

In making amino acid changes, the hydropathic index of amino acids may be considered. The importance of the hydropathic amino acid index in conferring interactive function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. It also is understood in the art that the substitution of like amino acids can be made effectively on the basis of hydrophilicity. The following hydrophilicity values can be assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still produce a functionally equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. However, in some aspects, a non-conservative substitution is contemplated. In certain aspects a random substitution is also contemplated. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Proteinaceous compositions may be made by any technique known to those of skill in the art, including (i) the expression of proteins, polypeptides, or peptides through standard molecular biological techniques, (ii) the isolation of proteinaceous compounds from natural sources, or (iii) the chemical synthesis of proteinaceous materials.

Amino acid sequence variants of polypeptides or polypeptide segments of these compositions can be substitutional, insertional, or deletion variants. A modification in a polypeptide may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250 or more non-contiguous or contiguous amino acids of a peptide or polypeptide.

Proteins may be recombinant or synthesized in vitro. Alternatively, a recombinant protein may be isolated from bacteria or host cell.

The term “functionally equivalent codon” is used herein to refer to codons that encode the same amino acid, such as the six codons for arginine or serine, and refers to codons that encode biologically equivalent amino acids.

It also will be understood that amino acid and nucleic acid sequences may include additional residues, such as additional N- or C-terminal amino acids, or 5′ or 3′ nucleic acid sequences, respectively, and yet still be essentially as set forth in one of the sequences disclosed herein, so long as the sequence meets the criteria set forth above, including the maintenance of protein activity. The addition of terminal sequences particularly applies to nucleic acid sequences that may, for example, include various non-coding sequences flanking either of the 5′ or 3′ portions of the coding region.

The polypeptides described herein may be fused, conjugated, or operatively linked to a label or tag. As used herein, the term “label” or “tag” intends a directly or indirectly detectable compound or composition that is conjugated directly or indirectly to the composition to be detected, e.g., polynucleotide or protein to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component involved in binding, or indirectly using a luminophore or fluorophore associated with another (e.g., reporter or indicator) component.

Examples of luminescent labels that produce signals include but are not limited to bioluminescence and chemiluminescence. Detectable luminescence response generally comprises a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

### II. NUCLEIC ACIDS, VECTORS AND RECOMBINANT HOST CELLS

A further object of the present invention relates to a nucleic acid sequence encoding for a polypeptide or a fusion protein according to the invention.

As used herein, a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

These nucleic acid sequences can be obtained by conventional methods well known to those skilled in the art. Typically, said nucleic acid is a DNA or RNA molecule, which may be included in a suitable vector, such as a plasmid, cosmid, episome, artificial chromosome, phage or viral vector.

So, a further object of the present invention relates to a vector and an expression cassette in which a nucleic acid molecule encoding for a polypeptide or a fusion protein of the invention is associated with suitable elements for controlling transcription (in particular promoter, enhancer and, optionally, terminator) and, optionally translation, and also the recombinant vectors into which a nucleic acid molecule in accordance with the invention is inserted. These recombinant vectors may, for example, be cloning vectors, or expression vectors.

As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like. Other examples of plasmids include replicating plasmids comprising an origin of replication, or integrative plasmids, such as for instance pUC, pcDNA, pBR, and the like. Other examples of viral vectors include adenoviral, retroviral, herpes virus and AAV vectors. Such recombinant viruses may be produced by techniques known in the art, such as by transfecting packaging cells or by transient transfection with helper plasmids or viruses.

A further aspect of the invention relates to a host cell comprising a nucleic acid molecule encoding for a polypeptide or a fusion protein according to the invention or a vector according to the invention. In particular aspects, a subject of the present invention is a prokaryotic or eukaryotic host cell genetically transformed with at least one nucleic acid molecule or vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

In some embodiments, for expressing and producing polypeptides or fusion proteins of the invention, prokaryotic cells, in particular E. coli cells, will be chosen. Actually, according to the invention, it is not mandatory to produce the polypeptide or the fusion protein of the invention in a eukaryotic context that will favor post-translational modifications (e.g. glycosylation). Furthermore, prokaryotic cells have the advantages to produce protein in large amounts. If a eukaryotic context is needed, yeasts (e.g. saccharomyces strains) may be particularly suitable since they allow production of large amounts of proteins. Otherwise, typical eukaryotic cell lines such as CHO, BHK-21, COS-7, C127, PER.C6, YB2/0 or HEK293 could be used, for their ability to process to the right post-translational modifications of the fusion protein of the invention.

The construction of expression vectors in accordance with the invention, and the transformation of the host cells can be carried out using conventional molecular biology techniques. The polypeptide or the fusion protein of the invention, can, for example, be obtained by culturing genetically transformed cells in accordance with the invention and recovering the polypeptide or the fusion protein expressed by said cell, from the culture. They may then, if necessary, be purified by conventional procedures, known in themselves to those skilled in the art, for example by fractional precipitation, in particular ammonium sulfate precipitation, electrophoresis, gel filtration, affinity chromatography, etc. In particular, conventional methods for preparing and purifying recombinant proteins may be used for producing the proteins in accordance with the invention.

A further aspect of the invention relates to a method for producing a polypeptide or a fusion protein of the invention comprising the step consisting of: (i) culturing a transformed host cell according to the invention under conditions suitable to allow expression of said polypeptide or fusion protein; and (ii) recovering the expressed polypeptide or fusion protein.

### III. KITS

Certain embodiments are directed to glycosylase detection kits. In general the glycosylase detection kits of the invention will include a hybrid glycosylase and/or a hyTDG-lyase as described herein. Optionally, the kit can include a substrate polynucleotide(s). The kit can preferably contain all buffer constituents and reagents for performing the respective assay.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Measurement of Deaminated Cytosine Adducts in DNA Using a Novel Hybrid Thymine DNA Glycosylase

A. Results

Construction and characterization of a hybrid human-thermophile mispaired thymine DNA glycosylase (hyTDG). A DNA sequence was constructed containing a His-tag (MGHHHHHH (SEQ ID NO:177)), a sequence encoding a 29 amino acid sequence derived from the amino terminus of the human TDG (SKKSGKSAKSKEKQEKITDTFKVKRKVDR (SEQ ID NO:2)) (25), and the catalytic core of tTDG (SEQ ID NO:3)(26-28). The amino acid sequence is shown in FIG. 2 (SEQ ID NO:1) and the DNA sequence is shown as FIG. 9 (SEQ ID NO:4).

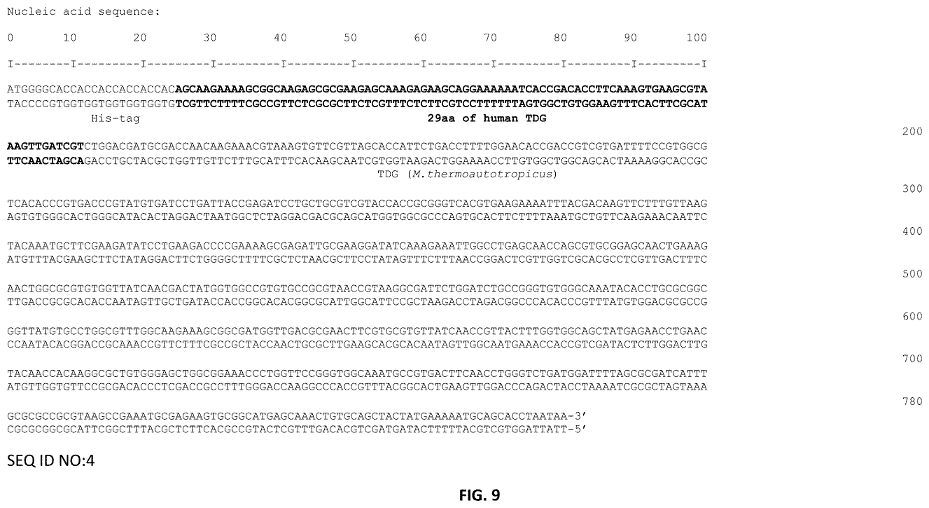

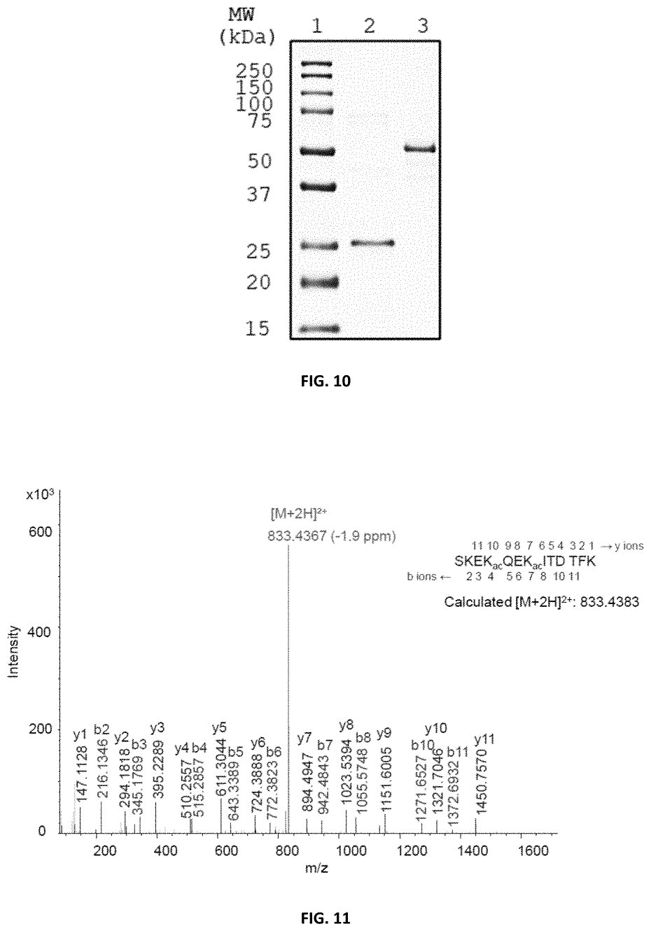

The plasmid encoding this sequence was cloned into BL3 competent cells and induced. The proteins isolated from the cell extract were fractionated and the His-tagged protein was isolated using a Ni²⁺ column. The isolated protein was analyzed by gel electrophoresis. The predominant band had an apparent molecular weight of 26.5 kDA (FIG. 10).

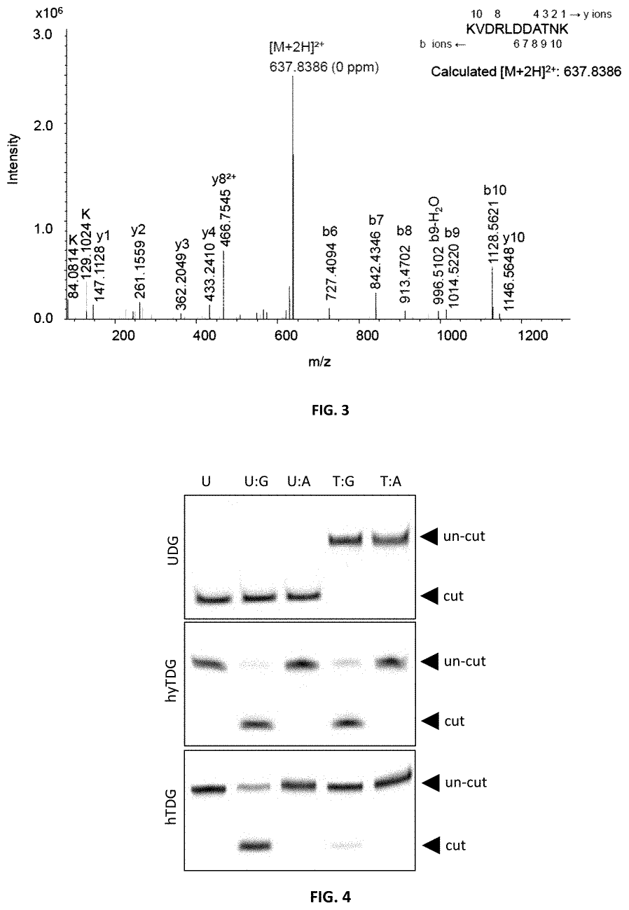

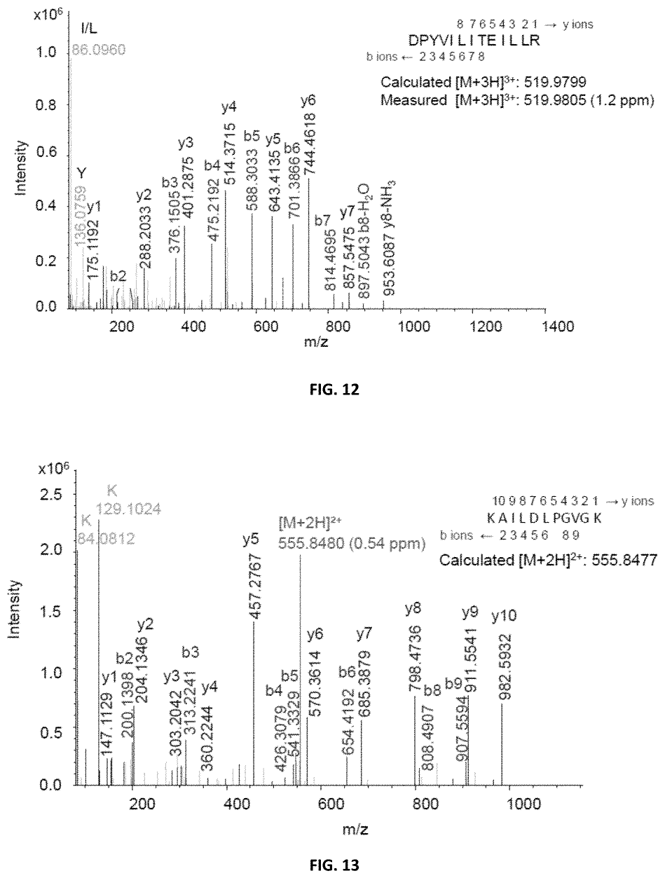

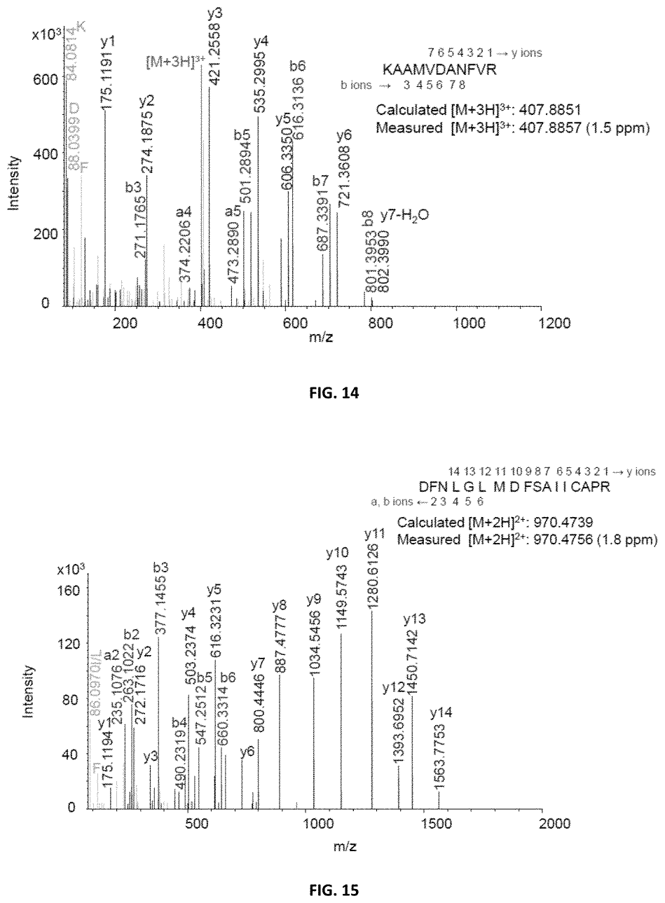

The purified protein was characterized by LC-MS/MS proteomic methods. A list of observed peptide fragments is provided in Table 1. The observed peptide fragments include KVDR/LDDATNK (SEQ ID NO:39) (amino acids 32-42) which is the junction between the human sequence and the tTDG catalytic core and is shown in FIG. 3. Several other significant fragments (26-29) were observed as well. The peptide SKEKQEKITDTFK (SEQ ID NO:21) (amino acids 16-28) is derived from the human 29 amino acid sequence (FIG. 11). Peptide DPYVILITEILLRR (SEQ ID NO:19) (amino acids 69-74) contains the “R” base flipper for this class of glycosylase (FIG. 12) and the peptide KAILDLPGVGK (SEQ ID NO:26) (amino acids 150-160, FIG. 13) which contains the LPGVGKY (SEQ ID NO:172) helix-hairpin-helix (HhH). The HhH motif consists of two α-helices flanking a β-hairpin with the conserved LPGVGX(K/S) (SEQ ID NO:173) which binds the DNA backbone non-specifically (28). The HhH motif places the thermophile TDG (tTDG) in the same class with the Mut Y and Endo III glycosylases. Peptide KAAMVDRNFVR (SEQ ID NO:24) (amino acids 173-183, FIG. 14) contains a conserved aspartic acid residue (bold) common to Mut Y, Endo III and tTDG glycosylases which is catalytic and interacts with the C1′ position of the target 2′-deoxyribose. Peptide DFNLGLMDFSAIICAPR (SEQ ID NO:174) (amino acids 219-235, FIG. 14) contains the first cysteine residue of the iron-sulfur cluster (FIG. 15) common to Mut Y, EndoIII and several other glycosylases.

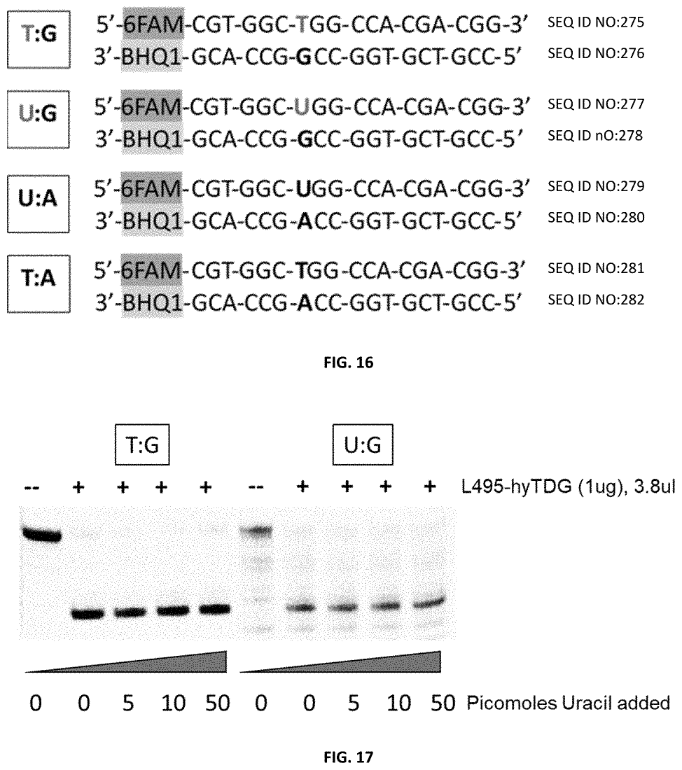

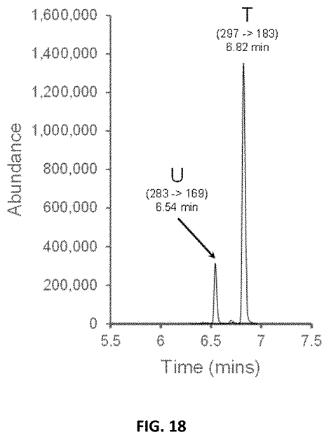

Examination of the activity of hyTDG using a real-time fluorescence assay. The activity of the purified hyTDG was first examined using an oligonucleotide cleavage assay. The hyTDG was incubated with a series of oligonucleotide duplexes containing U:A, U:G, T:A or T:G and a 5′-6FAM label. Duplexes containing defined sequences oligonucleotide sequences (FIG. 15) were incubated with glycosylases for defined time periods at specified temperatures. UDG was obtained from NEB. Human TDG (hTDG) was prepared as described previously (30). As shown in FIG. 4, UDG cleaves uracil from a single-stranded oligonucleotide as well as U:A and U:G, but not thymine-containing base pairs. The hyTDG cleaves both the U:G and T:G mispairs, but not U:A or T:A base pairs. In contrast, the hTDG efficiently cleaves uracil from a U:G mispair, but has little if any activity versus T:G. The gel assay was also used to determine if released uracil would inhibit hyTDG cleavage. At concentrations of uracil up to 50 pmol, ten times the amount of uracil in an oligonucleotide cleavage assay, no reduction of cleavage was observed (FIG. 16)

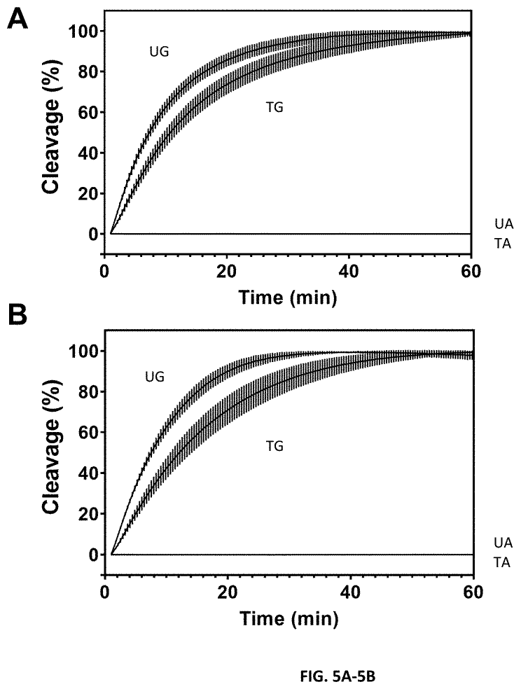

Cleavage was analyzed using a real-time fluorescence assay (31,32) with 5′-6FAM oligos duplexed with a complementary strand containing a 3′-BHQ1 quencher (FIG. 15). In this assay, glycosylase cleavage generates an abasic site which is then cleaved chemically using, N,N-dimethylethylenediamine (DMDA) (33), separating the 5′-6FAM from the quencher and allowing continuous monitoring of the fluorescence intensity. Cleavage of the U:G duplex by hyTDG reached 50% completion in 6.8+/−0.2 min whereas cleavage of the T:G duplex was somewhat slower, where 50% cleavage was observed at 9.8+/−0.2 min (FIG. 5A). Each reaction was run in triplicate. The average of all three runs is shown as a single line and error bars represent the standard deviation at each time point.

The inventors also sought to determine if an increase in DNA concentration decreased the observed cleavage rate. An excess of calf thymus DNA (20 μg) was added to the fluorescent probes, and the reaction was re-examined for the U:A, U:G, T:A and T:G duplexes. No cleavage of U:A or T:A oligonucleotides was observed under any conditions. Although the amount of DNA, based upon concentration of base pairs, was increased by a factor of ˜200, remarkably, the time required to cleave 50% of the U:G duplex decreased by roughly 1 min to 5.8+/−0.1 min and slightly increased by 1 min to 10.8+/−0.3 min for T:G (FIG. 5B).

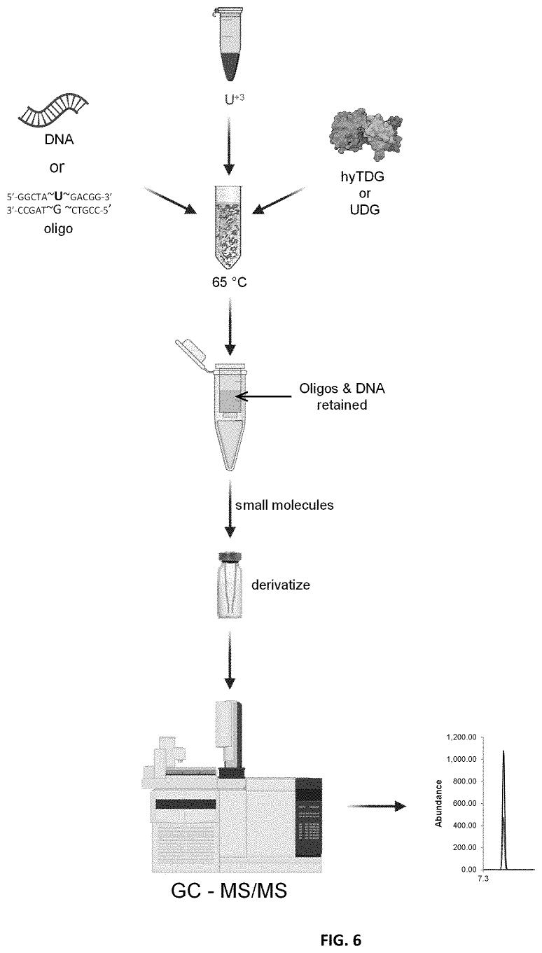

Examination of pyrimidines released from oligonucleotides and DNA by hyTDG. The above assays allow the examination of hyTDG activity against defined substrates. However, a more robust assay would involve hyTDG activity against multiple substrates simultaneously. An approach was developed that separates free bases from oligonucleotides or DNA using a spin filter. Isolated free bases can be chemically derivatized with tert-butydimethylsilyl groups and analyzed by GC-MS/MS. This workflow is shown schematically in FIG. 6.

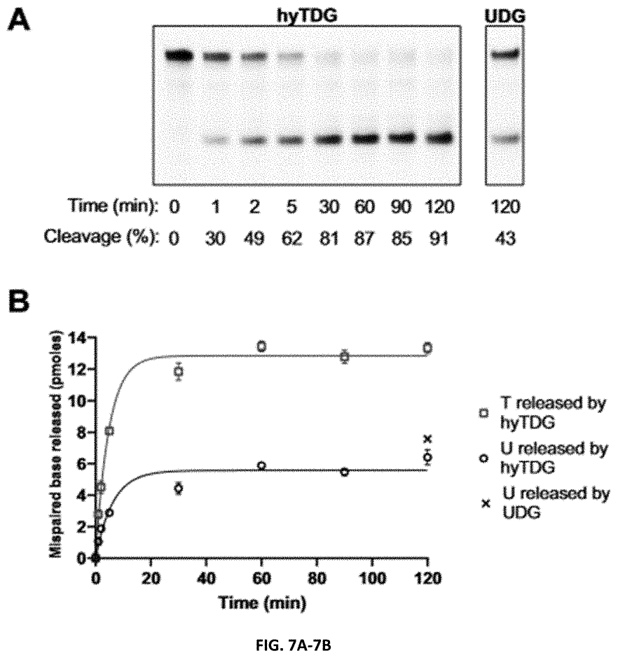

This approach was applied to a mixture of duplex oligonucleotides containing T:G and U:G mispairs in a 2 to 1 ratio. A mixture of 8.3 pmol U:G duplex, 16.7 pmol T:G duplex, and 250 pmol hyTDG with U+3 and T+4 standards in a volume of 25 μl was incubated at 65° C. for up to 120 min. The progress of the hyTDG reaction was followed simultaneously using both gel and GC-MS/MS methods (FIG. 7). A volume of 5 μl was used for the gel assay and 20 μl for the GC-MS/MS assay. Each time point was analyzed three times by GC-MS/MS.

As shown in FIG. 7A, approximately 91% of the mispaired duplexes were cleaved in 120 min as measured by the gel assay. Base release was also monitored by GC-MS/MS analysis (FIG. 7B). Consistent with the gel assay, cleavage of both U and T appeared to plateau after 60 min. At 120 min, 6.42±0.49 pmol of U was released and 13.33±0.34 pmol of T was released. The amount of U and T released is consistent with the amount of U and T oligonucleotides in the reaction. As a control, the oligonucleotide mixture was also incubated with UDG at 37° C. for 120 min. Gel analysis indicated 43% cleavage, slightly higher than the 33% expected based upon the composition of the mixture. The amount of U released by UDG as measured by GC-MS/MS was 7.58±0.20 pmol, also slightly higher than expected.

In a final series of experiments, the content of mispairs in calf thymus DNA was examined. First, calf thymus DNA was digested with the EcoRI restriction endonuclease to reduce its viscosity. Next, a portion of the calf thymus DNA was hydrolyzed in formic acid and the base composition examined by GC-MS using stable isotope-enriched standards of C, T, and 5-methylcytosine (5-mC). The base composition was observed to be 0.52±0.04 nmol C, 0.78 0.02 nmol T, and 0.03±0.0002 nmol 5-mC per microgram of calf thymus DNA.

To measure the content of U:G and T:G mispairs, a solution of EcoR1-digested calf thymus DNA (400 μg) containing isotope-enriched T+4 (14.5 pg T+4/μg DNA) and U+3 (5 pg/μg DNA) was incubated with either UDG (37° C.) or hyTDG (65° C.) for 90 min. Released free bases were separated from DNA and enzymes by spin filtration. Filtrates were dried, and the pyrimidine composition was measured by two analytical approaches. In the first approach, pyrimidines released by the glycosylases were converted to the TBDMS derivatives and analyzed by GC-MS/MS. In the second approach, pyrimidines were converted to the 3,5-bis(trifluoromethyl)benzyl bromide derivatives and analyzed by GC-MS using negative chemical ionization (GC-NCI-MS). All measurements for each approach represent three independent experiments.

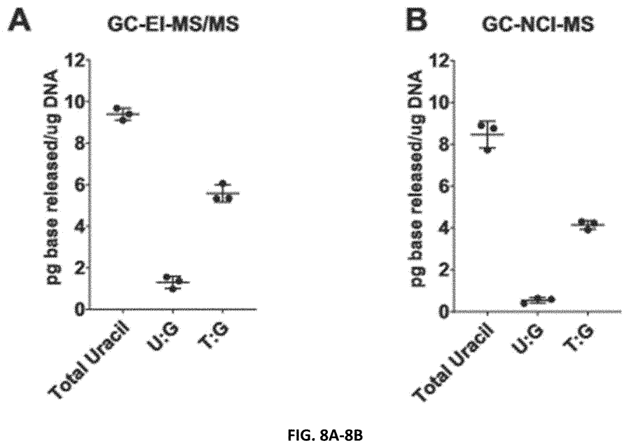

Incubation with UDG releases uracil in U:A and U:G base pairs as well as in single-stranded DNA. Total uracil in the calf thymus DNA released by UDG was 9.39±0.29 pg/μg DNA by GC-MS/MS (FIG. 8A). The amount of U released from U:G mispairs by hyTDG was 1.30±0.29, and the amount of T:G released from T:G mispairs was 5.58±0.42 pg/μg DNA.

The amount of U and T released was also measured using GC-NCI-MS (FIG. 8B). The amount of U released by UDG was measured to be 8.46±0.63 pg/μg DNA. The amount of U released by hyTDG was measured to be 0.54±0.13 pg/μg DNA, and the amount of T released was measured to be 4.14±0.21 pg/μg DNA.

The experiments depicted in FIGS. 7 and 8 were conducted in the presence of U+3 and T+4 internal standards for GC-MS analysis. To ensure that the internal standards did not inhibit base excision by hyTDG, we conducted a gel-based assay under similar conditions except that up to 50 pmol of U free base was added. The additional U free base had no observable effect upon glycosylase cleavage under the conditions of this experiment.

B. Material and Methods

Stable isotope standards. Enriched cytosine (C+2, ²H₂ H5, H6) and enriched 5-methylcytosine (5mC+4, methyl-²H₃, H6) were obtained from CDN isotopes (Quebec Canada). Enriched thymine (T+4, methyl ²H₃, ²H6) was obtained from Cambridge Isotope Laboratories (Tewksbury, Mass.). Enriched uracil (U+3, ¹⁵N₂, ¹³C2) was obtained from SigmaAldrich (Burlington, Mass.).

Construction, cloning and purification of the hybrid TDG (hyTDG). A DNA sequence was constructed with an amino terminal His-tag(6×His-tag), joined to the sequence encoding a 29 amino acid peptide from human TDG (hTDG, amino acids 82-112, NM_003211.6, SEQ ID NO:2) and the full-length thymine DNA glycosylase from M. thermoautotrophicus (tTDG, Orf 10, WP_010889848.1, SEQ ID NO:3). This hybrid DNA sequence was inserted into the pET-28a(+) expression vector between the NcoI and XhoI restriction sites. The hybrid DNA sequence is shown in FIG. 9 (SEQ ID NO:4) and the corresponding amino acid sequence of the hybrid TDG (hyTDG) in FIG. 2.

The pET-28a(+)-hyTDG plasmid was transformed into E. coli strain BL21 (DE3). Transformants were selected on an agar plate containing kanamycin. Selected clones were grown in 100 mL LB broth supplemented with kanamycin and induced with isopropyl β-D-1-thiogalactopyranoside (IPTG) for 6 h at 30° C. Cells were harvested by centrifugation at 4,100 rpm for 5 min and stored at −20° C. until used. Cell pellets were thawed and suspended in 4 mL lysis buffer (50 mM potassium phosphate, 20 mM imidazole, 3000 mM sodium chloride, 10 mM Q-mercaptoethanol, 1% triton and 1 mM phenylmethylsulfonyl fluoride (PMSF) and sonicated for 8 cycles, 30 sec each with 30 sec breaks on ice.

Supernatants were then centrifuged (12,000 rpm, 10 min), loaded onto previously equilibrated nickel-charged resin (HisPur Ni-NTA resin, ThermoFisher Scientific #88221), and incubated for 1.5 h at 4° C. The resin and supernatant were centrifuged on a column at 1000×g and washed as recommended by the vendor. The bound His-tagged protein was eluted with buffer (50 mM potassium phosphate, 300 mM sodium chloride, 10 mM β-mercaptoethanol, 100 mM imidazole). Total protein concentration was measured with a Bradford protein bioassay. Isolated protein was analyzed on a 12% tris-glycine polyacrylamide gel stained with Coomassie blue (FIG. 10) which indicated an apparent molecular weight of 26.5 kDa.

Characterization of the purified hyTDG by LC-MS/MS analysis. Approximately 10 μg of recombinant hyTDG was purified by sodium dodecyl sulfate polyacrylamide gel electrophoresis (SDS-PAGE). Gel bands were cut from the gel, destained with 50% methanol in water and dried. Gel bands were resuspended in 50 μL acetic anhydride and 200 μL acetic acid to chemically acetylate protein lysine residues. After incubation at 37° C. for 1 h, liquid was removed, and gel bands were washed three times with 1 mL deionized water. Gel bands were then dried and ground into a fine powder. Ammonium bicarbonate solution (100 μL, 50 mM) was added and the pH of the resulting gel was increased to approximately 8 with aqueous ammonia. Trypsin was then added, and the proteins were digested overnight at 37° C. Tryptic peptides were extracted with acetonitrile, dried, and resuspended in 50 μL of 1% formic acid for LC/MS/MS analysis.

Tryptic peptides were loaded onto a reversed-phase ProteoPre™ column loaded with Waters 5μ XSelect™ HSS T3 resin and Waters YMC ODS-AQ S-5 100 A resin and eluted with a gradient of acetonitrile in 0.1% formic acid. The LC column was directly interfaced with a QExactive™ mass analyzer which acquired data at a resolution of 35,000 in full scan mode and 17,500 in MS/MS mode. The topmost intense peptides in each MS survey were selected for MS/MS analysis. Peptides were identified with the PEAKS™ 8.5 software for de novo peptide sequencing. Acetylation of lysine (K), serine (S), threonine (T), cysteine (C), tyrosine (Y), and histidine (H) as well as oxidation of methionine (M) and deamination of asparagine (N) and glutamine (Q) were set as variable modifications.

Gel-based cleavage assay. A series of oligonucleotides were constructed containing a central pyrimidine, X, (cytosine (C), uracil (U) or thymine (T) paired opposite a purine (P), adenine (A) or guanine (G). One sequence [5′-6FAM-CGTGGCXGGCCACGACGG-3′ (SEQ ID NO:178)] contained the fluorophore, 6-carboxyfluorescein (6FAM) on the 5′ end. The complementary strand [5′-CCGTCGTGGCCPGCCACG (SEQ ID NO:179)] was synthesized with and without the 3′-BHQ1 black hole fluorescence quencher 1 (BHQ1) synthesized with 4′-(2-Nitro-4-toluyldiazo)-2′-methoxy-5′-methyl-azobenzene-4″-(N-ethyl-2-O-(4,4′-dimethoxytrityl))-N-ethyl-2-O-glycolate-linked controlled pore glass resin.

In a typical assay examined by gel electrophoresis, 2.5 pmol of 5′-6FAM-labelled oligonucleotide and two equivalents of an unlabeled complementary sequence were incubated in 10 μL buffer (10 mM potassium phosphate, 30 mM sodium chloride 40 mM potassium chloride) with UDG (5 units, E. coli, New England Biolabs), hyTDG (1 μg) or hTDG (1.5 μg) for 1 h at either 37° C. or 65° C. The reaction was terminated and the phosphate backbone of the oligonucleotide containing an abasic site was cleaved with 2 μL, 1 M NaOH at 95° C. for 10 min. Formamide (10 μL) was then added and the reaction mixture was loaded onto a 6 M urea denaturing 20% polyacrylamide gel. The oligonucleotide mixture was separated by electrophoresis for 45 min. Gels containing fluorescent bands were visualized and quantified on a Storm 860 phosphorimager.

Real-time fluorescence assay. In a typical real-time florescence assay, 25 pmol of 5′-6FAM labelled oligonucleotide was annealed with 50 pmol of the complementary sequence containing the 3′-BHQ1 quencher in a 25 μL reaction volume containing 10 mM potassium phosphate buffer, pH 7.7, 30 mM NaCl, 40 mM KCl. To ensure cleavage of the phosphate backbone following glycosylase release of a target base, N,N-dimethylethylenediamine (DMDA, 100 mM final concentration) was added. The reaction was initiated upon the addition of the glycosylase and fluorescence was monitored at 65° C. every 20 s in a Roche 480 qPCR instrument. Real-time fluorescence assays were acquired in triplicate. Graphs of data were prepared with PRISM software.

Oligonucleotide cleavage assays monitored by gel and GC-MS/MS. Cleavage assays monitored by GC-MS/MS were identical to those used for gel electrophoresis assay but scaled up by a factor of 5. From each reaction, 5 μL was taken for gel electrophoresis while 20 μL was diluted to 400 μL with water and spin-filtered (Amiconm Ultra Ultracel 3k, #UFC500396) at 14,000×g for 45 min. The eluate was added to a GC vial with 5-ethyluracil (EtU) as an internal standard and isotope enriched uracil (U+3) and thymine (T+4) and dried under reduced pressure.

Pyrimidines were converted to their tert-butyl dimethylsilyl derivatives in acetonitrile and 0.5 μL of the reaction solution was injected onto an Agilent 7890 GC containing an HP-5 column. The GC oven temperature was held constant at 100° C. for 2 min, ramped to 260° C. at 30° C./min and held at that temperature for 10 min. The GC was directly coupled to an Agilent 7000C triple quadrupole detector. The most predominant ions of both uracil (283 amu, rt 6.54 min) and thymine (297 amu, rt 6.82 min) derivatives correspond to the M-57 (tert-butyl) fragment. The corresponding loss of 114 amu is the transition used to monitor both pyrimidines.

Preparation of calf thymus DNA and analysis of base composition. Calf thymus DNA was dissolved in buffer containing 5 mM NaCl, 1 mM tris pH 7, 1 mM MgCl₂ and 0.1 mM DDT. DNA (˜50 mg) was digested with ˜20,000 units of EcoRI endonuclease (New England Biolabs) at 37° C. for 4 h to reduce viscosity (61). Digested DNA was precipitated with ammonium acetate/ethanol, resuspended in water, and dialyzed overnight.

A portion of the digested calf thymus DNA was hydrolyzed in 88% formic acid at 140° C. for 40 min. Isotope-enriched standards of thymine (T+4), cytosine (C+2), and 5-methylcytosine (5mC+3) at a ratio of 20:1 (C/5mC) were added to the vials which were then evaporated to dryness under reduced pressure. Bases were converted to the TBDMS derivatives in acetonitrile at 140° C. for 40 min. Samples were injected onto an Agilent 7890A GC containing a DB5 column. The initial GC oven temperature was 100° C. for 2 min, ramped to 260° C. at 30° C. per min then held at 260° C. for 10 min. The GC was directly interfaced to an Agilent 5975C mass selective detector and data was collected in the selected ion mode. Molar amounts of C and T were determined by comparing experimental peak areas to standard curves. The molar amount of 5mC was determined by comparing peak areas of unenriched C and 5mC to peak areas of the isotope enriched standards. Base composition determinations were done in triplicate.

Analysis of bases released from calf thymus DNA by hyTDG. EcoRI digested DNA was dissolved in buffer optimized for either UDG or hyTDG as described above. For studies with calf thymus DNA, an isotope enriched standard of uracil was added (¹⁵N₂ ¹³C-uracil, U+3) Following incubation at 37° C. (UDG) or 65° C. (hyTDG) for 90 min, Enzyme reactions were diluted with water and spin filtered as above. The column flow-through was dried under reduced pressure in vials containing 5-ethyluracil as an internal standard. Free bases were converted to the TBDMS derivatives and injected onto the GC-triple quad. As described above, uracil, thymine, and the U+³ standard were monitored using selected transitions. Molar amounts of uracil and thymine were determined by comparison of peak areas with the peak area of the U+3 internal standard.

Abbreviations used include: hyTDG, hybrid thymine DNA glycosylase; hTDG, human TDG glycosylase; UDG, uracil DNA glycosylase; tTDG, thymine DNA glycosylase from Methanobacterium thermoautotrophicum; 6FAM, 6-carboxyfluorescein; BHQ1, black whole quencher 1; GC-MS/MS, gas chromatography-tandem mass spectrometry; LC-MS/MS, liquid chromatography tandem mass spectrometry; TBDMS, tert-butydimethylsilyl; DMIDA, N,N-dimethyethylenediamine; EtU, 5-ethyluracil.

TABLE 1
List of peptides identified for hyTDG

| Peptide | −10 lgP | Mass | Length | ppm | m/z | RT |
|---|---|---|---|---|---|---|
| HTRDPYVILITEILLRR (SEQ ID NO: 5) | 170 | 2107.2 | 17 | −4.1 | 527.8 | 71.6 |
| HTRDPYVILITEILLR (SEQ ID NO: 6) | 144 | 1951.1 | 16 | −4.6 | 651.4 | 76.0 |
| YFGGSYENLNYNHK(+42.01)ALWELAETLVPGGK (SEQ | 141 | 3211.6 | 28 | −2.8 | 1071.5 | 73.7 |
| ID NO: 7) | | | | | | |
| YFGGSYEN(+.98)LNYNHK(+42.01)ALWELAETLVPGGK | 138 | 3212.5 | 28 | −1.4 | 1071.9 | 75.6 |
| (SEQ ID NO: 7) | | | | | | |
| DPYVILITEILLR (SEQ ID NO: 8) | 138 | 1556.9 | 13 | −3.4 | 779.5 | 89.9 |
| KVFVSTILTFWNTDR (SEQ ID NO: 9) | 125 | 1826.0 | 15 | −2.3 | 609.7 | 67.9 |
| VVINDYGGR (SEQ ID NO: 10) | 123 | 991.5 | 9 | −1.4 | 496.8 | 36.6 |
| YFGGSYENLNYNHK (SEQ ID NO: 11) | 122 | 1704.8 | 14 | −2.6 | 853.4 | 45.0 |
| AILDLPGVGK(+42.01)YTC AAVM(+15.99)CLAFGK | 119 | 3684.9 | 34 | −11.8 | 1229.3 | 81.7 |
| (+42.01)K(+42.01)AAMVDANFVR (SEQ ID NO: 12) | | | | | | |
| YFGGSYENLNYNHK(+42.01) (SEQ ID NO: 11) | 118 | 1746.8 | 14 | −4.2 | 874.4 | 49.1 |
| VFVSTILTFWNTDR (SEQ ID NO: 13) | 118 | 1697.9 | 14 | −3 | 849.9 | 73.0 |
| YFGGSYENLNYN(+.98)HK (SEQ ID NO: 11) | 118 | 1705.7 | 14 | -0.6 | 569.6 | 45.5 |
| YFGGSYEN(+.98)LN(+.98)YNHK(+42.01)ALWELAETLVP | 117 | 3213.5 | 28 | 1.3 | 1072.2 | 76.2 |
| GGK (SEQ ID NO: 7) | | | | | | |
| VVIN(+.98)DYGGR (SEQ ID NO: 10) | 114 | 992.5 | 9 | −2.5 | 497.3 | 38.7 |
| AAMVDANFVR (SEQ ID NO: 14) | 114 | 1092.5 | 10 | −1.8 | 547.3 | 46.8 |
| ALWELAETLVPGGK (SEQ ID NO: 15) | 112 | 1482.8 | 14 | −7.9 | 742.4 | 92.7 |
| TPK(+42.01)SEIAK(+42.01)DIK(+42.01)EIGLSNQR | 111 | 2252.2 | 19 | −2.2 | 1127.1 | 67.3 |
| (SEQ ID NO: 16) | | | | | | |
| YFGGSYENLN(+.98)YNHK (SEQ ID NO: 11) | 107 | 1705.7 | 14 | -0.5 | 569.6 | 46.0 |
| YFGGSYENLNYN(+.98)HK(+42.01)ALWELAETLVPGGK | 107 | 3212.5 | 28 | 2 | 1071.9 | 74.0 |
| (SEQ ID NO: 7) | | | | | | |
| AAM(+15.99)VDANFVR (SEQ ID NO: 17) | 106 | 1108.5 | 10 | −2 | 555.3 | 42.8 |
| TPK(+42.01)SEIAKDIK(+42.01)EIGLSNQR | 105 | 2210.2 | 19 | −3.4 | 737.7 | 63.2 |
| (SEQ ID NO: 16) | | | | | | |
| YFGGSYEN(+.98)LN(+.98)YN(+.98)HK(+42.01) | 105 | 3214.5 | 28 | 6.6 | 1072.5 | 74.4 |
| ALWELAETLVPGGK (SEQ ID NO: 7) | | | | | | |
| K(+42.01)VFVSTILTFWNTDR (SEQ ID NO: 26) | 104 | 1868.0 | 15 | −3.4 | 623.7 | 75.7 |
| SEIAK(+42.01)DIK(+42.01)EIGLSNQR (SEQ ID NO: 18) | 102 | 1884.0 | 16 | −5 | 943.0 | 58.0 |
| DPYVILITEILLRR (SEQ ID NO: 19) | 102 | 1713.0 | 14 | −3 | 572.0 | 81.7 |
| TRDPYVILITEILLR (SEQ ID NO: 20) | 101 | 1814.1 | 15 | −4.3 | 605.7 | 80.3 |
| SEIAK(+42.01)DIK(+42.01)EIGLSN(+.98)QR | 101 | 1885.0 | 16 | −3.4 | 943.5 | 57.5 |
| (SEQ ID NO: 30) | | | | | | |
| VFVSTILTFWN(+.98)TDR (SEQ ID NO: 13) | 100 | 1698.9 | 14 | 4.3 | 850.4 | 74.1 |
| YFGGSYEN(+.98)LNYNHK (SEQ ID NO: 11) | 100 | 1705.7 | 14 | −7 | 853.9 | 46.1 |
| YFGGS YENLN(+.98) YN(+.98)HK(+42.01)ALWELAETLVP | 98 | 3213.5 | 28 | 10 | 1072.2 | 73.4 |
| GGK (SEQ ID NO: 7) | | | | | | |
| TPK(+42.01)SEIAK(+42.01)DIK(+42.01)EIGLSN(+.98)QR | 97 | 2253.2 | 19 | −1.6 | 752.1 | 66.8 |
| (SEQ ID NO: 16) | | | | | | |
| AILDLPGVGK (SEQ ID NO: 196) | 97 | 981.6 | 10 | −3.6 | 491.8 | 83.0 |
| SK(+42.01)EKQEK(+42.01)ITDTFK (SEQ ID NO: 21) | 95 | 1664.9 | 13 | −12.9 | 833.4 | 42.1 |
| TTAGHVK(+42.01)K(+42.01)IYDK(+42.01)FFVK (SEQ ID | 95 | 2007.1 | 16 | -0.2 | 670.0 | 61.5 |
| NO: 22) | | | | | | |
| VDRLDDATNK (SEQ ID NO: 23) | 94 | 1145.6 | 10 | −3.7 | 382.9 | 27.4 |
| AILDLPGVGK(+42.01)YTCAAVMCLAFGK(+42.01)K(+42. | 93 | 3684.9 | 34 | −9.6 | 922.2 | 82.8 |
| 01)AAM(+15.99)VDANFVR (SEQ ID NO: 12) | | | | | | |
| YFGGS YEN(+.98)LNYN(+.98)HK(+42.01) ALWELAETLVP | 92 | 3213.5 | 28 | 1.9 | 1072.2 | 74.7 |
| GGK (SEQ ID NO: 7) | | | | | | |
| KAAMVDANFVR (SEQ ID NO: 24) | 92 | 1220.6 | 11 | −5.2 | 407.9 | 41.7 |
| SKEK(+42.01)QEK(+42.01)ITDTFK (SEQ ID NO: 21) | 91 | 1664.9 | 13 | −2.4 | 833.4 | 44.7 |
| SK(+42.01)EK(+42.01)QEK(+42.01)ITDTFK | 90 | 1706.9 | 13 | −3.6 | 854.4 | 49.3 |
| (SEQ ID NO: 21) | | | | | | |
| K(+42.01)AAMVDANFVR (SEQ ID NO: 44) | 89 | 1262.6 | 11 | −1.7 | 632.3 | 48.4 |
| TTAGHVK(+42.01)K(+42.01)IYDK (SEQ ID NO: 25) | 88 | 1443.8 | 12 | −3.5 | 722.9 | 37.4 |
| K(+42.01)AILDLPGVGK (SEQ ID NO: 26) | 88 | 1151.7 | 11 | −2.9 | 576.9 | 55.1 |
| YFGGSYENLNYNH (SEQ ID NO: 27) | 88 | 1576.7 | 13 | −5.6 | 789.3 | 48.1 |
| KVFVSTILTFWNTDRR (SEQ ID NO: 28) | 88 | 1982.1 | 16 | −4.6 | 496.5 | 63.4 |
| EK(+42.01)QEK(+42.01)ITDTFK(+42.01)VK | 88 | 1718.9 | 13 | −4.3 | 860.5 | 51.6 |
| (SEQ ID NO: 29) | | | | | | |
| EKQEK(+42.01)ITDTFK(+42.01)VK (SEQ ID NO: 29) | 87 | 1676.9 | 13 | -0.6 | 839.5 | 46.8 |
| K(+42.01)VFVSTILTFWN(+.98)TDR (SEQ ID NO: 51) | 86 | 1869.0 | 15 | 4.7 | 624.0 | 76.3 |
| EKQEK(+42.01)ITDTFK (SEQ ID NO: 30) | 86 | 1407.7 | 11 | −2.9 | 704.9 | 40.5 |
| VFVSTILTFWNTDRR (SEQ ID NO: 31) | 85 | 1854.0 | 15 | −4.3 | 619.0 | 68.7 |
| KVFVSTILTFWN(+.98)TDR (SEQ ID NO: 9) | 83 | 1827.0 | 15 | −3.2 | 610.0 | 67.1 |
| KAAM(+15.99)VDANFVR (SEQ ID NO: 24) | 83 | 1236.6 | 11 | -0.5 | 413.2 | 37.7 |
| KAILDLPGVGK (SEQ ID NO: 26) | 83 | 1109.7 | 11 | −3.5 | 555.8 | 49.7 |
| EK(+42.01)QEK(+42.01)ITDTFK (SEQ ID NO: 57) | 82 | 1449.7 | 11 | −2 | 725.9 | 47.0 |
| K(+42.01)AAM(+15.99)VDANFVR (SEQ ID NO: 58) | 81 | 1278.6 | 11 | −3.2 | 640.3 | 43.7 |
| GK(+42.01)K(+42.01)AAMVDANFVR (SEQ ID NO: 32) | 81 | 1489.8 | 13 | −4.4 | 745.9 | 50.9 |
| YTCAAVMCLAFGK(+42.01)K(+42.01)AAMVDANFVR | 80 | 2663.3 | 24 | −6.1 | 888.8 | 80.0 |
| (SEQ ID NO: 33) | | | | | | |
| YFGGSYEN(+.98)LNYNHK(+42.01) (SEQ ID NO: 11) | 79 | 1747.7 | 14 | 6.8 | 874.9 | 49.1 |
| VSTILTFWNTDR (SEQ ID NO: 34) | 78 | 1451.7 | 12 | −1.2 | 726.9 | 63.8 |
| K(+42.01)VFVSTILTFWNTDRR (SEQ ID NO: 63) | 78 | 2024.1 | 16 | −4.2 | 675.7 | 70.8 |
| VVINDYGG (SEQ ID NO: 35) | 77 | 835.4 | 8 | −5.6 | 836.4 | 41.3 |
| DIK(+42.01)EIGLSNQR (SEQ ID NO: 36) | 77 | 1313.7 | 11 | −3.1 | 657.9 | 46.6 |
| QEK(+42.01)ITDTFK(+42.01)VK(+42.01)R | 76 | 1617.9 | 12 | −5.7 | 809.9 | 50.6 |
| (SEQ ID NO: 37) | | | | | | |
| WELAETLVPGGK (SEQ ID NO: 38) | 76 | 1298.7 | 12 | −6.8 | 650.3 | 68.3 |
| K(+42.01)VDRLDDATNK (SEQ ID NO: 39) | 75 | 1315.7 | 11 | −4.7 | 439.6 | 31.7 |
| AAM(+15.99)VDAN(+.98)FVR (SEQ ID NO: 69) | 75 | 1109.5 | 10 | 16.4 | 555.8 | 42.1 |
| QEK(+42.01)ITDTFK (SEQ ID NO: 40) | 75 | 1150.6 | 9 | -0.8 | 1151.6 | 44.1 |
| K(+42.01)VDRLDDATNK(+42.01)K (SEQ ID NO: 41) | 75 | 1485.8 | 12 | −2.8 | 496.3 | 35.2 |
| IYDK(+42.01)FFVK(+42.01)YK (SEQ ID NO: 42) | 75 | 1433.8 | 10 | −4.2 | 717.9 | 59.1 |
| KIYDK(+42.01)FFVK(+42.01)YK (SEQ ID NO: 43) | 75 | 1561.9 | 11 | 0.3 | 521.6 | 56.2 |
| YTC AAVM(+15.99)CL AFGK(+42.01)K(+42.01) AAMVDA | 74 | 2679.3 | 24 | −10 | 894.1 | 72.4 |
| NFVR (SEQIDNO: 33) | | | | | | |
| QEK(+42.01)ITDTFK(+42.01)VK (SEQ ID NO: 44) | 73 | 1419.8 | 11 | −4.4 | 710.9 | 49.9 |
| ITDTFK(+42.01)VK(+42.01)R (SEQ ID NO: 45) | 73 | 1190.7 | 9 | −3.5 | 397.9 | 45.5 |
| KIYDK(+42.01)FFVK (SEQ ID NO: 46) | 73 | 1228.7 | 9 | −4.8 | 410.6 | 52.3 |
| AEQLK(+42.01)ELAR (SEQ ID NO: 47) | 73 | 1098.6 | 9 | −2.9 | 550.3 | 45.4 |
| YFGGSYENLN(+.98)YNHK(+42.01)ALWELAETLVPGGK | 73 | 3212.5 | 28 | 9.2 | 1071.9 | 73.3 |
| (SEQ ID NO: 7) | | | | | | |
| EIGLSNQR (SEQ ID NO: 48) | 73 | 915.5 | 8 | −4.1 | 458.7 | 34.3 |
| TILTFWNTDR (SEQ ID NO: 49) | 72 | 1265.6 | 10 | −6.1 | 633.8 | 60.4 |
| SEIAKDIK(+42.01)EIGLSNQR (SEQ ID NO: 82) | 72 | 1842.0 | 16 | −5.3 | 615.0 | 53.0 |
| TPK(+42.01)SEIAK(+42.01)DIK (SEQ ID NO: 50) | 71 | 1312.7 | 11 | −1.8 | 657.4 | 47.2 |
| YFGGSYEN(+.98)LN(+.98)YNHK (SEQ ID NO: 11) | 70 | 1706.7 | 14 | 9.6 | 569.9 | 46.3 |
| AILDLPGVGK(+42.01)YTCAAVMCLAFGK (SEQ ID | 69 | 2382.2 | 23 | −9.3 | 795.1 | 77.2 |
| NO: 51) | | | | | | |
| ILTFWNTDR (SEQ ID NO: 52) | 69 | 1164.6 | 9 | −2 | 583.3 | 57.4 |
| TILTFWNTDRR (SEQ ID NO: 53) | 69 | 1421.7 | 11 | −3.9 | 474.9 | 55.7 |
| LDDATNK(+42.01)K (SEQ ID NO: 54) | 69 | 945.5 | 8 | −1.9 | 473.7 | 25.7 |
| GK(+42.01)K(+42.01)AAM(+15.99)VDANFVR | 68 | 1505.8 | 13 | −2.6 | 753.9 | 46.1 |
| (SEQ ID NO: 32) | | | | | | |
| IYDK(+42.01)FFVK (SEQ ID NO: 55) | 68 | 1100.6 | 8 | −3.4 | 551.3 | 55.3 |
| IAK(+42.01)DIK(+42.01)EIGLSNQR (SEQ ID NO: 56) | 68 | 1667.9 | 14 | −2.6 | 835.0 | 51.3 |
| LDDATNK(+42.01)K(+42.01)R (SEQ ID NO: 57) | 67 | 1143.6 | 9 | −2.7 | 572.8 | 32.3 |
| DIK(+42.01)EIGLSN(+.98)QR (SEQ ID NO: 36) | 66 | 1314.7 | 11 | -0.3 | 658.3 | 47.1 |
| AILDLPGVGK(+42.01)YTCAAVMCLAFGK(+42.01)K | 66 | 3685.8 | 34 | −3.6 | 1229.6 | 82.7 |
| (+42.01)AAM(+15.99)VDAN(+.98)FVR (SEQ ID NO: 12) | | | | | | |
| LDDATNK (SEQ ID NO: 58) | 66 | 775.4 | 7 | −4.2 | 388.7 | 20.5 |
| SK(+42.01)EK(+42.01)Q(+.98)EK(+42.01)ITDTFK | 66 | 1707.9 | 13 | 9.8 | 854.9 | 49.0 |
| (SEQ ID NO: 96) | | | | | | |
| VVINDYGGRVPR (SEQ ID NO: 59) | 66 | 1343.7 | 12 | −2 | 448.9 | 40.0 |
| K(+42.01) AILDLPGVGK(+42.01) YTCAAVMCLAFGK | 65 | 2552.3 | 24 | −13.3 | 1277.2 | 77.1 |
| (SEQ ID NO: 60) | | | | | | |
| K(+42.01)VFVSTILTFWN(+.98)TDRR (SEQ ID NO: 99) | 65 | 2025.1 | 16 | 1.3 | 676.0 | 71.7 |
| EKQ(+.98)EK(+42.01)ITDTFK(+42.01)VK (SEQ ID NO: 29) | 64 | 1677.9 | 13 | 13 | 840.0 | 46.6 |
| K(+42.01) AILDLPGVGK(+42.01) YTCAAVM(+15.99)CLAF | 63 | 2568.3 | 24 | −7 | 857.1 | 72.3 |
| GK (SEQ ID NO: 60) | | | | | | |
| EIGLSNQ (SEQ ID NO: 61) | 63 | 759.4 | 7 | −4.4 | 760.4 | 37.9 |
| FFVK(+42.01)YK (SEQ ID NO: 62) | 63 | 872.5 | 6 | −3.5 | 437.2 | 47.4 |
| SAK(+42.01)SK(+42.01)EK(+42.01)QEK (SEQ ID NO: 63) | 63 | 1287.7 | 10 | −3.5 | 644.8 | 29.2 |
| LDDATN(+.98)K(+42.01)K(+42.01)R (SEQ ID NO: 57) | 63 | 1144.6 | 9 | −1.9 | 573.3 | 31.2 |
| EK(+42.01)Q(+.98)EK(+42.01)ITDTFK (SEQ ID NO: 106) | 62 | 1450.7 | 11 | 10 | 726.4 | 46.6 |
| LDDATNKK (SEQ ID NO: 54) | 62 | 903.5 | 8 | −1.2 | 452.7 | 19.2 |
| ITDTFK(+42.01)VK (SEQ ID NO: 64) | 62 | 992.6 | 8 | −2.2 | 993.6 | 43.1 |
| ITDTFK (SEQ ID NO: 65) | 62 | 723.4 | 6 | −3.5 | 724.4 | 34.2 |
| LDDATN(+.98)K(+42.01)KR (SEQ ID NO: 57) | 61 | 1102.6 | 9 | −1.4 | 552.3 | 26.0 |
| VDRLDDATNK(+42.01)K (SEQ ID NO: 66) | 61 | 1315.7 | 11 | −1.3 | 439.6 | 32.5 |
| SEIAK(+42.01)DIK (SEQ ID NO: 67) | 61 | 944.5 | 8 | −1.3 | 945.5 | 37.3 |
| LDDATNK(+42.01)KR (SEQ ID NO: 57) | 61 | 1101.6 | 9 | −1.9 | 368.2 | 25.7 |
| RDFPWR (SEQ ID NO: 68) | 60 | 875.4 | 6 | −2.4 | 438.7 | 45.7 |
| MVDANFVR (SEQ ID NO: 69) | 60 | 950.5 | 8 | −3.4 | 476.2 | 43.5 |
| K(+42.01)IYDK(+42.01)FFVK (SEQ ID NO: 116) | 60 | 1270.7 | 9 | −3.7 | 636.4 | 58.6 |
| IYDKFFVK (SEQ ID NO: 55) | 60 | 1058.6 | 8 | −3.7 | 353.9 | 47.5 |
| TPK(+42.01)SEIAKDIK(+42.01)EIGLSN(+.98)QR | 60 | 2211.2 | 19 | 5.1 | 738.1 | 63.5 |
| (SEQ ID NO: 16) | | | | | | |
| TPK(+42.01)SEIAK (SEQ ID NO: 70) | 60 | 914.5 | 8 | 3.9 | 458.3 | 31.3 |
| TTAGHVK(+42.01)K (SEQ ID NO: 71) | 59 | 882.5 | 8 | −2.4 | 442.3 | 20.4 |
| EIGLSN(+.98)QR (SEQ ID NO: 48) | 58 | 916.5 | 8 | −2 | 459.2 | 35.4 |
| K(+42.01)AAMVDAN(+.98)FVR (SEQ ID NO: 122) | 58 | 1263.6 | 11 | −6.2 | 632.8 | 49.6 |
| TFWNTDR (SEQ ID NO: 72) | 58 | 938.4 | 7 | −3.9 | 470.2 | 44.7 |
| K(+42.01)AILDLPGVGK(+42.01)YT (SEQ ID NO: 73) | 57 | 1457.8 | 13 | 0.4 | 729.9 | 65.4 |
| TPK(+42.01)SEIAK(+42.01)DIK(+42.01)EIGLSN | 57 | 2254.2 | 19 | −7.2 | 1128.1 | 67.5 |
| (+.98)Q(+.98)R (SEQ ID NO: 16) | | | | | | |
| DFNLGLMDF (SEQ ID NO: 74) | 57 | 1070.5 | 9 | −1.2 | 1071.5 | 75.9 |
| K(+42.01)AILDLPGVGK(+42.01)Y (SEQ ID NO: 75) | 57 | 1356.8 | 12 | −3.3 | 679.4 | 65.9 |
| SGK(+42.01)SAK(+42.01)SK(+42.01)EK (SEQ ID NO: 76) | 57 | 1174.6 | 10 | -0.2 | 588.3 | 28.6 |
| LDDATNKK(+42.01)R (SEQ ID NO: 57) | 57 | 1101.6 | 9 | −3.8 | 551.8 | 24.9 |
| SEIAK(+42.01)DIK(+42.01)EIGLSN(+.98)Q(+.98)R | 56 | 1886.0 | 16 | 12.2 | 944.0 | 58.3 |
| (SEQ ID NO: 130) | | | | | | |
| VSTILTFWNTDRR (SEQ ID NO: 77) | 56 | 1607.8 | 13 | −1.4 | 537.0 | 59.2 |
| VILITEILLR (SEQ ID NO: 78) | 56 | 1181.8 | 10 | −5.6 | 591.9 | 72.6 |
| VDANFVR (SEQ ID NO: 79) | 56 | 819.4 | 7 | −3.3 | 410.7 | 38.4 |
| IYDKFFVK(+42.01)YK (SEQ ID NO: 42) | 56 | 1391.7 | 10 | −5.2 | 464.9 | 52.2 |
| K(+42.01)VDRLDDATNKK (SEQ ID NO: 41) | 55 | 1443.8 | 12 | −5.5 | 482.3 | 29.3 |
| KIYDKFFVK (SEQ ID NO: 46) | 54 | 1186.7 | 9 | −2.2 | 396.6 | 45.1 |
| TTAGHVK(+42.01)K(+42.01)IYDK(+42.01)F | 54 | 1632.9 | 13 | -0.1 | 817.4 | 55.9 |
| (SEQ ID NO: 80) | | | | | | |
| EIGLSN (SEQ ID NO: 81) | 54 | 631.3 | 6 | −1.2 | 632.3 | 37.5 |
| LCSYYEK (SEQ ID NO: 82) | 54 | 904.4 | 7 | −9.4 | 453.2 | 31.5 |
| IK(+42.01)EIGLSNQR (SEQ ID NO: 83) | 54 | 1198.7 | 10 | −2.2 | 600.3 | 42.8 |
| YFGGS YENLNYNHK(+42.01) ALWEL AETL VPGGK(+42.01) | 53 | 3253.6 | 28 | 3.1 | 814.4 | 77.1 |
| (SEQ ID NO: 7) | | | | | | |
| TTAGHVK (SEQ ID NO: 84) | 53 | 712.4 | 7 | −5.9 | 357.2 | 12.6 |
| AMVDANFVR (SEQ ID NO: 85) | 53 | 1021.5 | 9 | −3.3 | 511.8 | 46.0 |
| SEIAKDIK(+42.01)EIGLSN(+.98)QR (SEQ ID NO: 144) | 53 | 1843.0 | 16 | 8.3 | 922.5 | 53.1 |
| ILTFWN(+.98)TDR (SEQ ID NO: 145) | 52 | 1165.6 | 9 | 6.2 | 583.8 | 58.2 |
| VFVSTILTF (SEQ ID NO: 86) | 52 | 1025.6 | 9 | −3.2 | 513.8 | 71.8 |
| VSTILTFWN(+.98)TDR (SEQ ID NO: 34) | 51 | 1452.7 | 12 | 11.2 | 727.4 | 63.9 |
| K(+42.01)AILDLPGVGK(+42.01)YTCAAVMCLAF (SEQ | 51 | 2367.2 | 22 | −10.5 | 790.1 | 85.1 |
| ID NO: 87) | | | | | | |
| K(+42.01)SGK(+42.01)SAK(+42.01)SK (SEQ ID NO: 88) | 51 | 1045.6 | 9 | −2.2 | 523.8 | 25.7 |
| SGK(+42.01)SAK(+42.01)SK (SEQ ID NO: 89) | 51 | 875.5 | 8 | −3.4 | 438.7 | 22.3 |
| DFPWR (SEQ ID NO: 90) | 51 | 719.3 | 5 | -0.9 | 720.3 | 50.9 |
| YTCAAVMCLAFGK (SEQ ID NO: 91) | 51 | 1376.6 | 13 | −16.2 | 689.3 | 65.2 |
| ITEILLR (SEQ ID NO: 92) | 50 | 856.5 | 7 | −4.4 | 429.3 | 49.3 |
| M(+15.99)VDANFVR (SEQ ID NO: 154) | 50 | 966.5 | 8 | −10.1 | 484.2 | 39.9 |
| AEQLK (SEQ ID NO: 93) | 49 | 587.3 | 5 | −5.3 | 588.3 | 18.9 |
| VK(+42.01)K(+42.01)IYDK(+42.01)FFVK (SEQ ID NO: 94) | 49 | 1539.9 | 11 | −8.1 | 770.9 | 62.4 |
| TFWNTDRR (SEQ ID NO: 95) | 48 | 1094.5 | 8 | −1.5 | 365.8 | 39.9 |
| K(+42.01)VFVSTIL (SEQ ID NO: 96) | 48 | 947.6 | 8 | −1.3 | 948.6 | 63.6 |
| CGM(+15.99)SKLCSYYEK (SEQ ID NO: 97) | 47 | 1426.6 | 12 | −3.6 | 476.5 | 32.2 |
| KVDRLDDATNK (SEQ ID NO: 39) | 47 | 1273.7 | 11 | −3.7 | 425.6 | 26.3 |
| TPK(+42.01)SEIAKDIK(+42.01)EIGLSN(+.98)Q(+.98)R | 47 | 2212.2 | 19 | 4.3 | 738.4 | 62.9 |
| (SEQ ID NO: 16) | | | | | | |
| ILTFWNTDRR (SEQ ID NO: 98) | 47 | 1320.7 | 10 | −5.5 | 441.2 | 52.8 |
| SEIAK (SEQ ID NO: 99) | 47 | 546.3 | 5 | −2.1 | 547.3 | 16.2 |
| KVFVSTILTF (SEQ ID NO: 100) | 46 | 1153.7 | 10 | −3.2 | 577.8 | 65.1 |
| AAM(+15.99)VDAN (SEQ ID NO: 101) | 46 | 706.3 | 7 | −3.8 | 707.3 | 28.8 |
| AAMVDAN (SEQ ID NO: 101) | 46 | 690.3 | 7 | −5.2 | 691.3 | 28.1 |
| YFGGSY (SEQ ID NO: 102) | 46 | 692.3 | 6 | −6.3 | 693.3 | 45.2 |
| TLVPGGK (SEQ ID NO: 103) | 45 | 670.4 | 7 | −2.9 | 671.4 | 68.5 |
| TILTFWN(+.98)TDR (SEQ ID NO: 49) | 45 | 1266.6 | 10 | 8.3 | 634.3 | 60.2 |
| K(+42.01)VFVSTILTF (SEQ ID NO: 170) | 45 | 1195.7 | 10 | −4 | 598.8 | 73.4 |
| VFVSTILTFWN(+.98)TDRR (SEQ ID NO: 31) | 45 | 1855.0 | 15 | 5.3 | 619.3 | 68.4 |
| KSGK(+42.01)SAK(+42.01)SK (SEQ ID NO: 88) | 45 | 1003.6 | 9 | −4.3 | 502.8 | 18.8 |
| VFVSTILTFW (SEQ ID NO: 104) | 44 | 1211.7 | 10 | −2.6 | 606.8 | 82.6 |
| K(+42.01)VDRLDDATN (SEQ ID NO: 105) | 44 | 1187.6 | 10 | -0.9 | 594.8 | 34.1 |
| WINDY (SEQ ID NO: 106) | 43 | 721.4 | 6 | −2 | 722.4 | 43.3 |
| DLPGVGK (SEQ ID NO: 107) | 43 | 684.4 | 7 | -0.8 | 685.4 | 54.4 |
| SAK(+42.01)SK(+42.01)EK (SEQ ID NO: 108) | 43 | 860.5 | 7 | −7.8 | 431.2 | 22.7 |
| SK(+42.01)EK(+42.01)QEK (SEQ ID NO: 109) | 42 | 959.5 | 7 | −4.3 | 480.8 | 23.0 |
| WNTDR (SEQ ID NO: 110) | 41 | 690.3 | 5 | −4 | 691.3 | 26.2 |
| K(+42.01)AAM(+15.99)VDAN(+.98)FVR (SEQ ID NO: 180) | 41 | 1279.6 | 11 | 3.5 | 640.8 | 44.1 |
| AEQLK(+42.01) (SEQ ID NO: 93) | 41 | 629.3 | 5 | −5.4 | 630.3 | 33.0 |
| KAAMVDAN(+.98)FVR (SEQ ID NO: 24) | 41 | 1221.6 | 11 | 16.2 | 611.8 | 42.2 |
| FEDILK (SEQ ID NO: 111) | 41 | 763.4 | 6 | −4.1 | 382.7 | 46.4 |
| TDTFK (SEQ ID NO: 112) | 41 | 610.3 | 5 | −1 | 611.3 | 34.2 |
| K(+42.01)SGK(+42.01)SAK (SEQ ID NO: 113) | 40 | 788.4 | 7 | −3.5 | 395.2 | 19.9 |
| K(+42.01)PK(+42.01)CEKCGMSK (SEQ ID NO: 114) | 40 | 1321.6 | 11 | −17.5 | 441.5 | 31.8 |
| DFNLGL (SEQ ID NO: 115) | 40 | 677.3 | 6 | −3.3 | 678.3 | 61.5 |
| VDRLDDATNKK (SEQ ID NO: 66) | 39 | 1273.7 | 11 | −5.3 | 425.6 | 26.1 |
| IGLSNQR (SEQ ID NO: 116) | 39 | 786.4 | 7 | −4.4 | 394.2 | 33.3 |
| LDLPGVGK (SEQ ID NO: 117) | 39 | 797.5 | 8 | −4.2 | 798.5 | 54.5 |
| TTAGHVK(+42.01) (SEQ ID NO: 84) | 38 | 754.4 | 7 | −5.8 | 378.2 | 21.6 |
| KAAM(+15.99)VDAN(+.98)FVR (SEQ ID NO: 24) | 38 | 1237.6 | 11 | 13.1 | 619.8 | 37.6 |
| VFVSTIL (SEQ ID NO: 118) | 37 | 777.5 | 7 | −3.7 | 778.5 | 61.6 |
| K(+42.01)Q(+.98)EK(+42.01)IIDTFK (SEQ ID NO: 119) | 37 | 1321.7 | 10 | 1.2 | 441.6 | 33.7 |
| TT AGH VK(+42.01)K(+42.01)IYDK(+42.01)FFVK | 36 | 2049.1 | 16 | 4.7 | 684.0 | 65.9 |
| (+42.01) (SEQ ID NO: 22) | | | | | | |
| LDDATN (SEQ ID NO: 120) | 36 | 647.3 | 6 | −5.6 | 648.3 | 22.5 |
| VDRLDDATNK(+42.01)K(+42.01)R (SEQ ID NO: 121) | 36 | 1513.8 | 12 | −2.7 | 505.6 | 35.7 |
| K(+42.01)ELARVVINDYGGR (SEQ ID NO: 122) | 36 | 1630.9 | 14 | −15.9 | 544.6 | 51.4 |
| K(+42.01)IYDK (SEQ ID NO: 123) | 36 | 707.4 | 5 | 1.3 | 708.4 | 28.8 |
| KAAMVDAN (SEQ ID NO: 124) | 36 | 818.4 | 8 | −2 | 410.2 | 24.5 |
| VINDYGGR (SEQ ID NO: 125) | 36 | 892.4 | 8 | −8 | 447.2 | 29.8 |
| SGKSAK (SEQ ID NO: 126) | 35 | 576.3 | 6 | 16.4 | 577.3 | 25.9 |
| AILDLPGVGK(+42.01)YTCAAVMCLAFGK(+42.01)K | 34 | 3669.8 | 34 | 3.6 | 918.5 | 86.4 |
| (+42.01)AAMVDAN(+.98)FVR (SEQ ID NO: 12) | | | | | | |
| HTRDPY (SEQ ID NO: 127) | 34 | 787.4 | 6 | −3.6 | 394.7 | 22.5 |
| LDDATNK(+42.01) (SEQ ID NO: 58) | 34 | 817.4 | 7 | −3.1 | 409.7 | 26.7 |
| YFGGSYEN(+.98)LNYNHK(+42.01)ALWELAETLVPGGK | 33 | 3254.6 | 28 | 5.5 | 1085.9 | 77.8 |
| (+42.01) (SEQ ID NO: 7) | | | | | | |
| QEK(+42.01)ITDTFK(+42.01) (SEQ ID NO: 40) | 33 | 1192.6 | 9 | -0.6 | 597.3 | 48.8 |
| K(+42.01)VFVST (SEQ ID NO: 128) | 32 | 721.4 | 6 | 1 | 722.4 | 44.2 |
| EKQEK(+42.01)ITDTF (SEQ ID NO: 129) | 32 | 1279.6 | 10 | −6.3 | 640.8 | 46.9 |
| AILDLPGVGK(+42.01)YTC AAVM(+15.99)CLAFGK | 31 | 3685.8 | 34 | 1 | 922.5 | 81.7 |
| (+42.01)K(+42.01)AAMVDAN(+.98)FVR (SEQ ID NO: 12) | | | | | | |
| LDDATNK(+42.01)K(+42.01) (SEQ ID NO: 54) | 31 | 987.5 | 8 | −4.8 | 494.7 | 31.5 |
| ITDTF (SEQ ID NO: 130) | 31 | 595.3 | 5 | 0.6 | 596.3 | 43.5 |
| FWNTDR (SEQ ID NO: 131) | 30 | 837.4 | 6 | −2.9 | 419.7 | 38.9 |
| SGK(+42.01)SAK (SEQ ID NO: 126) | 29 | 618.3 | 6 | −5.5 | 619.3 | 13.5 |
| SEIAK(+42.01)DIKEIGLSN(+.98)QR (SEQ ID NO: 18) | 29 | 1843.0 | 16 | 4.6 | 615.3 | 53.2 |
| INDYGGR (SEQ ID NO: 132) | 29 | 793.4 | 7 | −3.7 | 397.7 | 37.2 |
| VK(+42.01)K(+42.01)IYDK (SEQ ID NO: 133) | 29 | 976.6 | 7 | -0.5 | 489.3 | 37.5 |
| RTTAGHVK(+42.01)K (SEQ ID NO: 134) | 28 | 1038.6 | 9 | −3.8 | 520.3 | 17.0 |
| FFVK(+42.01)Y (SEQ ID NO: 135) | 28 | 744.4 | 5 | −8 | 745.4 | 57.2 |
| VFVST (SEQ ID NO: 136) | 28 | 551.3 | 5 | −4.7 | 552.3 | 39.0 |
| AEQ(+.98)LK(+42.01)ELAR (SEQ ID NO: 47) | 27 | 1099.6 | 9 | 13.1 | 367.5 | 43.5 |
| AEQ(+.98)LK (SEQ ID NO: 93) | 26 | 588.3 | 5 | 15.2 | 589.3 | 35.1 |
| DATNK (SEQ ID NO: 137) | 26 | 547.3 | 5 | −3 | 548.3 | 20.0 |
| TAGHVK(+42.01)K (SEQ ID NO: 138) | 26 | 781.4 | 7 | −5 | 391.7 | 19.6 |
| FVK(+42.01)YK (SEQ ID NO: 139) | 25 | 725.4 | 5 | −2.4 | 363.7 | 35.7 |
| TPKSEIAK (SEQ ID NO: 70) | 24 | 872.5 | 8 | -0.2 | 437.3 | 29.8 |
| EIGLSNQ(+.98)RA (SEQ ID NO: 140) | 24 | 987.5 | 9 | -0.6 | 494.8 | 37.0 |
| TTAGH (SEQ ID NO: 141) | 24 | 485.2 | 5 | −4.1 | 486.2 | 7.4 |
| NDYGGR (SEQ ID NO: 142) | 23 | 680.3 | 6 | −4 | 681.3 | 36.9 |
| EIGLSNQRAEQLK (SEQ ID NO: 143) | 23 | 1484.8 | 13 | 10.3 | 743.4 | 80.9 |
| RK(+42.01)VDR (SEQ ID NO: 144) | 23 | 714.4 | 5 | −4.5 | 358.2 | 17.7 |
| DATNK(+42.01)K(+42.01)R (SEQ ID NO: 145) | 23 | 915.5 | 7 | −3.2 | 458.7 | 26.1 |
| WNTDRR (SEQ ID NO: 146) | 23 | 846.4 | 6 | −2.9 | 424.2 | 25.4 |
| LVPGGK (SEQ ID NO: 147) | 23 | 569.4 | 6 | −4.5 | 570.4 | 68.4 |
| VDRLDDATN(+.98)K (SEQ ID NO: 23) | 22 | 1146.6 | 10 | 14.2 | 383.2 | 28.0 |
| TAGHVKK(+42.01) (SEQ ID NO: 138) | 22 | 781.4 | 7 | −5 | 391.7 | 19.8 |
| K(+42.01)FFVK (SEQ ID NO: 148) | 22 | 709.4 | 5 | −18.5 | 355.7 | 39.8 |
| AILDLPGVGK(+42.01)YT (SEQ ID NO: 149) | 21 | 1287.7 | 12 | 7.8 | 644.9 | 63.6 |
| IGLSNQ(+.98)RAEQ(+.98)LK (SEQ ID NO: 150) | 21 | 1357.7 | 12 | 0.4 | 679.9 | 50.5 |
| YFGGS YENLNYNHK(+42.01) ALWELAETLVPGGKCRD | 21 | 3585.7 | 31 | 2.7 | 1196.2 | 74.3 |
| (SEQ ID NO: 151) | | | | | | |
| IN(+.98)DYGGR (SEQ ID NO: 132) | 21 | 794.4 | 7 | −2.9 | 795.4 | 38.7 |
| AEQLK(+42.01)EL (SEQ ID NO: 152) | 21 | 871.5 | 7 | −4.9 | 436.7 | 49.0 |
| FSAIICAPR (SEQ ID NO: 153) | 20 | 976.5 | 9 | 15.7 | 489.3 | 48.7 |
| SKEK(+42.01)QEK (SEQ ID NO: 109) | 20 | 917.5 | 7 | −4.6 | 459.7 | 13.3 |
| DIK(+42.01)EIGLSNQ(+.98)RAEQ(+.98)LK(+42.01) | 20 | 1927.0 | 16 | 6.4 | 643.3 | 71.2 |
| (SEQ ID NO: 154) | | | | | | |
| VPGGK (SEQ ID NO: 155) | 20 | 456.3 | 5 | −4 | 457.3 | 68.4 |
| RTTAGH (SEQ ID NO: 156) | 19 | 641.3 | 6 | 4.5 | 642.3 | 53.2 |
| K(+42.01)ELAR (SEQ ID NO: 157) | 19 | 657.4 | 5 | −8.6 | 658.4 | 43.4 |
| INRYFGGSYENLNYNHK(+42.01) (SEQ ID NO: 158) | 19 | 2130.0 | 17 | 1.5 | 1066.0 | 83.5 |
| K(+42.01)VFVS (SEQ ID NO: 159) | 19 | 620.4 | 5 | −2.8 | 621.4 | 43.1 |
| DYGGR (SEQ ID NO: 160) | 19 | 566.2 | 5 | 1.5 | 567.3 | 37.0 |
| KIYDK(+42.01)F (SEQ ID NO: 161) | 19 | 854.5 | 6 | −4 | 428.2 | 47.3 |
| DFSAIICAPR (SEQ ID NO: 162) | 18 | 1091.5 | 10 | 7.4 | 546.8 | 44.8 |
| GLSN(+.98)QR (SEQ ID NO: 163) | 18 | 674.3 | 6 | −12 | 675.3 | 47.9 |
| N(+.98)RKAILDLPGVGK (SEQ ID NO: 164) | 17 | 1380.8 | 13 | −7.9 | 1381.8 | 95.3 |
| IN(+.98)DYGGRVPR (SEQ ID NO: 165) | 17 | 1146.6 | 10 | 4.8 | 574.3 | 31.3 |
| HHHHHHSKK (SEQ ID NO: 166) | 16 | 1183.6 | 9 | 0.4 | 1184.6 | 98.9 |
| KSGK(+42.01)SAK (SEQ ID NO: 113) | 16 | 746.4 | 7 | −4.7 | 374.2 | 11.9 |
| DDATNKK (SEQ ID NO: 167) | 16 | 790.4 | 7 | −3.4 | 396.2 | 16.4 |
| K(+42.01)ELARVVIN(+.98)DYGGR (SEQ ID NO: 122) | 16 | 1631.9 | 14 | −3.8 | 545.0 | 51.1 |
| TPK(+42.01)SEIAK(+42.01) (SEQ ID NO: 70) | 16 | 956.5 | 8 | 3.3 | 479.3 | 34.7 |
| SK(+42.01)EK(+42.01)Q(+.98)EK (SEQ ID NO: 109) | 16 | 960.5 | 7 | −16.1 | 481.2 | 34.3 |
| EKQ(+.98)EK (SEQ ID NO: 168) | 16 | 661.3 | 5 | 17.1 | 662.3 | 34.1 |
| EQ(+.98)LKELAR (SEQ ID NO: 169) | 15 | 986.5 | 8 | −14.3 | 494.3 | 48.3 |
| PGVGK (SEQ ID NO: 170) | 15 | 456.3 | 5 | −2.8 | 457.3 | 49.6 |
| KVFVSTIL (SEQ ID NO: 96) | 15 | 905.6 | 8 | −3.4 | 453.8 | 54.9 |

### EXAMPLE 1 REFERENCES

1. Hollstein et al., Science. 1991 Jul. 5; 253(5015):49-53.
2. Magewu and Jones, Mol Cell Biol. 1994 June; 14(6):4225-32.
3. Iengar, Nucleic Acids Res. 2012 August; 40(14):6401-13.
4. Simon et al., Nucleic Acids Res 2017; 45 (D1): D777-D783.
5. Lewis et al., Proc Natl Acad Sci USA. 2016 Jul. 19; 113(29):8194-9.
6. Lindahl and Nyberg, Biochemistry. 1974 Jul. 30; 13(16):3405-10.
7. Coulondre et al., Nature. 1978 Aug. 24; 274(5673):775-80.
8. Duncan and Miller, Nature. 1980 Oct. 9; 287(5782):560-1.
9. Wang et al., Biochim Biophys Acta. 1982 Jun. 30; 697(3):371-7.
10. Shen et al., Nucleic Acids Res. 1994 Mar. 25; 22(6):972-6.
11. Cadet et al., Cold Spring Harb Perspect Biol. 2013 Feb. 1; 5(2).
12. Sangaraju et al., J Am Soc Mass Spectrom. 2014 July; 25(7):1124-35.
13. You et al., Acc Chem Res. 2016 Feb. 16; 49(2):205-13.
14. Jumpathong et al., Proc Natl Acad Sci USA. 2015 Sep. 1; 112(35):E4845-53.
15. Gates, Chem Res Toxicol. 2009 November; 22(11):1747-60.
16. Totsuka et al., Cancer Sci. 2021 January; 112(1):7-15.
17. Blount and Ames, Anal Biochem. 1994 June; 219(2):195-200.
18. Beckman et al., Free Radic Biol Med. 2000 August; 29(3-4):357-67.
19. Jaruga et al., Free Radic Biol Med. 2008 Dec. 15; 45(12):1601-9.
20. Mullins et al., Methods. 2013 November; 64(1):59-66.
21. Minko et al., DNA Repair (Amst). 2020 January; 85:102741.
22. Waters and Swann, J Biol Chem. 1998 Aug. 7; 273(32):20007-14.
23. Bennett et al., J Am Chem Soc. 2006 Sep. 27; 128(38):12510-9.
24. Liu et al., Chem Res Toxicol. 2002 August; 15(8):1001-9.
25. Coey et al., Nucleic Acids Res. 2016 Dec. 1; 44(21):10248-10258.
26. Horst and Fritz, EMBO J. 1996 Oct. 1; 15(19):5459-69.
27. Begley and Cunningham R P, Protein Eng. 1999 April; 12(4):333-40.
28. Mol et al., J Mol Biol. 2002 Jan. 18; 315(3):373-84.
29. Yoon et al., Nucleic Acids Res. 2003 Sep. 15; 31(18):5399-404.
30. Hardeland et al., J Biol Chem. 2000 Oct. 27; 275(43):33449-56.
31. Kladova et al., Biochemistry (Mosc). 2020 April; 85(4):480-489.
32. Mechetin et al., Int J Mol Sci. 2020 Apr. 28; 21(9):3118.
33. Mchugh and Knowland, Nucleic Acids Res. 23, 16664-16706.
34. Kirk, Biochem J. 1967 November; 105(2):673-7.
35. Sturm and Taylor, Nucleic Acids Res. 1981 Sep. 25; 9(18):4537-46.
36. Richards et al., Adv Enzyme Regul. 1984; 22:157-85.
37. Kavli et al., DNA Repair (Amst). 2007 Apr. 1; 6(4):505-16.
38. Olinski et al., Mutat Res. 2010 December; 705(3):239-45.
39. Dube et al., Biochim Biophys Acta. 1979 Feb. 27; 561(2):369-82.
40. Ivarie, Nucleic Acids Res. 1987 Dec. 10; 15(23):9975-83.
41. Pu and Struhl, Nucleic Acids Res. 1992 Feb. 25; 20(4):771-5.
42. Rogstad et al., Biochemistry. 2002 Jun. 25; 41(25):8093-102.
43. Mancini et al., Am J Hum Genet. 1997 July; 61(1):80-7.
44. Cooper et al., Hum Genomics. 2010 August; 4(6):406-10.
45. Poulos et al., Nucleic Acids Res. 2017 Jul. 27; 45(13):7786-7795.
46. Tornaletti and Pfeifer, Oncogene. 1995 Apr. 20; 10(8):1493-9.
47. Rideout et al., Science. 1990 Sep. 14; 249(4974):1288-90.
48. Jang et al., Genes (Basel). 2017 May 23; 8(6):148.
49. Dosanjh et al., Biochemistry, 30, 11595-11599.
50. Shen et al., Nucleic Acids Res., 20, 5119-25.
51. Sowers et al., Mutat Res. 1989 November; 215(1):131-8.
52. Ehrlich et al., Biosci Rep. 1986 April; 6(4):387-93.
53. Schmutte et al., Cancer Res. 1995 Sep. 1; 55(17):3742-6.
54. Briggs and Heyn, Methods Mol Biol. 2012; 840:143-54.
55. Do et al., Clin Chem. 2013 September; 59(9):1376-83.
56. Costello et al., Nucleic Acids Res. 2013 Apr. 1; 41(6):e67.
57. Arbeithuber et al., DNA Res. 2016 December; 23(6):547-559.
58. Kim et al., J Mol Diagn. 2017 January; 19(1):137-146.
59. Chen et al., Science. 2017 Feb. 17; 355(6326):752-756.
60. Kim et al., J Mol Diagn. 2017 January; 19(1):137-146.
61. Philippsen et al., Eur J Biochem. 1975 Sep. 1; 57(1):55-68.

### Example 2

### Characterization of a Novel Thermostable DNA Lyase

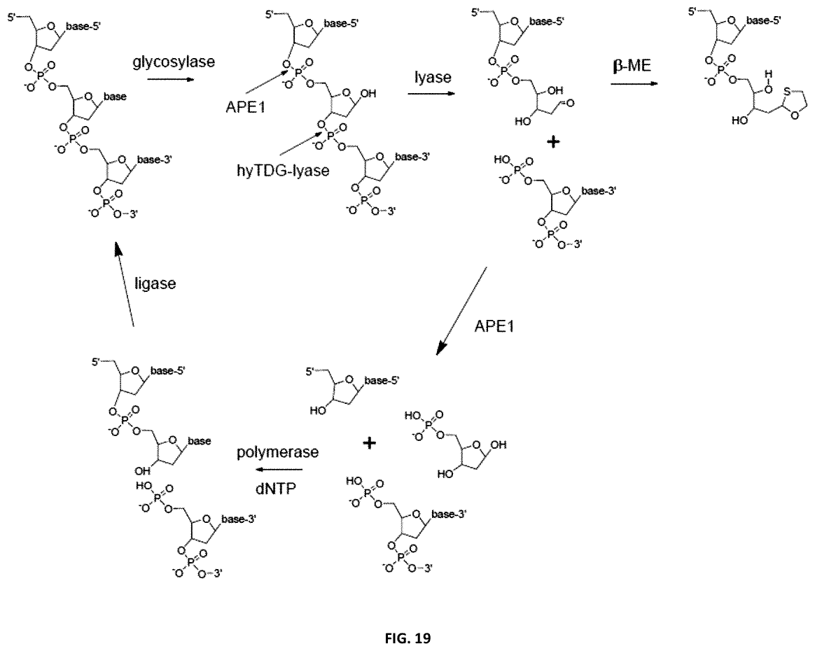

Substantial research efforts are currently focused on DNA repair enzymes because of the importance of DNA damage and repair to human disease. Most endogenous DNA damage is repaired by the base excision repair (BER) pathway (1-5). The BER pathway is initiated by a series of lesion-specific glycosylases that recognize and remove a damaged base from DNA. The resulting abasic site is then cleaved by a lyase domain connected to the glycosylase in the case of bifunctional glycosylases, or a separate lyase in the case of the monofunctional glycosylases. The repair cycle is then completed by insertion of one or more nucleotides by a DNA polymerase and the phosphodiester backbone is restored by a DNA ligase (FIG. 19).

In addition to understanding fundamentally important DNA repair pathways, glycosylases and other DNA repair proteins are potential pharmacological targets for the treatment of infectious diseases as well as tumors which overexpress DNA repair enzymes, particularly those resistant to chemotherapy or radiation (6-10). DNA repair enzymes are also of interest in the sequencing of DNA damage and in removing damage from DNA prior to next generation DNA sequencing (11-15).

The measurement of monofunctional glycosylase activity usually requires the cleavage of the DNA phosphodiester backbone following the glycosylase removal of a target base and the separation of cleaved oligonucleotides by gel electrophoresis or chromatography. The cleavage of oligonucleotides containing abasic sites can be accomplished using alkali, however, alkaline conditions can damage some modified bases including those that are the target of the glycosylase assay (16-19). Bifunctional glycosylases and apurinic-apyrimidinic (AP) endonucleases can also be used to cleave abasic sites generated by monofunctional glycosylases, however, finding experimental conditions including buffer composition and temperature that are simultaneously compatible with both enzymes presents a challenge.

Recently, a hybrid thymine DNA glycosylase, hyTDG (20) described herein, was created by combining a 29-amino acid sequence from the human TDG that enhances overall glycosylase activity (e.g., SEQ ID NO:2) (21) with the catalytic domain of the MIG (22-25). This glycosylase has activity against a broad range of uracil analogs mispaired with guanine. It was shown that a single amino acid change in MIG converted it from a glycosylase to a lyase (25). A Y163 to K163 substitution was inserted into a hyTDG to create a hyTDG-lyase. The data presented here demonstrates a hyTDG-lyase is active over a broad temperature range and is compatible with multiple buffer conditions.

A. Results

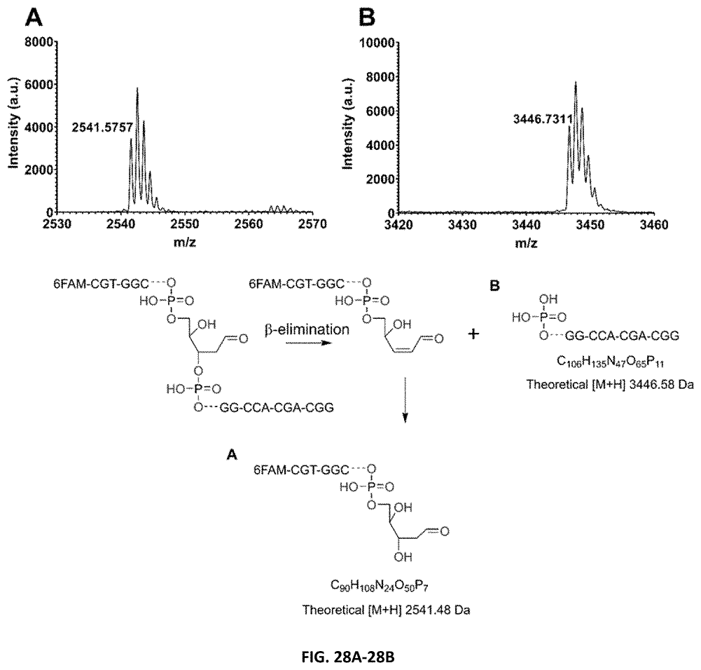

A Y163K mutant of a hybrid thymine DNA glycosylase (hyTDG) was constructed and is referred to as the hyTDG-lyase. The mutant protein had an apparent molecular weight of 26.5 kDa (FIG. 27). The amino acid sequence of the hyTDG-lyase is shown in FIG. 20A (SEQ TD NO:186 and SEQ ID NO:189). The amino acid sequence of hyTDG-lyase was confirmed by analysis of tryptic peptides by LC-MS/MS. One peptide, NRKAILDLPGVGKK (SEQ TD NO: 188), containing the 163K substitution is underlined in FIG. 20A. The corresponding mass spectrum of this peptide is shown in FIG. 19B. Several other peptides derived from hyTDG-lyase were observed and are listed in Table 2.

TABLE 2
Identified peptide sequences for hyTDG-lyase by MS.

| PEPTIDE | −10 lgP | Mass | Length | PPm | m/z |
|---|---|---|---|---|---|
| TPK(+42.01)SEIAK(+42.01)DIK(+42.01)EIGLSNQR (SEQ ID NO: 16) | 112.69 | 2252.2012 | 19 | −5.1 | 1127.1022 |
| SEIAK(+42.01)DIKEIGLSNQR (SEQ ID NO: 18) | 108.7 | 1841.9846 | 16 | −3.5 | 921.9964 |
| SEIAK(+42.01)DIK(+42.01)EIGLSNQR (SEQ ID NO: 18) | 101.19 | 1883.9952 | 16 | 1.3 | 943.0061 |
| SEIAKDIK(+42.01)EIGLSNQR (SEQ ID NO: 18) | 96.82 | 1841.9846 | 16 | 3.9 | 922.0032 |
| DFNLGLM(+15.99)DFSAIIC(+57.02)APR (SEQ ID NO: 174) | 87.55 | 1954.9281 | 17 | 2.5 | 652.6516 |
| YFGGSYENLNYNH(+42.01)K (SEQ ID NO: 11) | 86.22 | 1746.7638 | 14 | 2.9 | 874.3917 |
| SKEK(+42.01)QEK(+42.01)ITDTFK (SEQ ID NO: 21) | 86.06 | 1664.8621 | 13 | −1 | 833.4375 |
| VVINDYGGR (SEQ ID NO: 10) | 85.38 | 991.5087 | 9 | 2.2 | 496.7627 |
| DPYVILITS(+42.01)ILLR (SEQ ID NO: 8) | 85.17 | 1556.9177 | 13 | 4.9 | 779.47 |
| YFGGSYENLNY(+42.01)NHK (SEQ ID NO: 11) | 84.6 | 1746.7638 | 14 | 0.6 | 874.3897 |
| YFGGS(+42.01)YENLNYNHK (SEQ ID NO: 11) | 82.65 | 1746.7638 | 14 | -0.9 | 874.3884 |
| TTAGHVK(+42.01)K(+42.01)IYDK(+42.01)FFVK (SEQ ID NO: 22) | 79.81 | 2007.0829 | 16 | 4.8 | 670.0381 |
| AAMVDANFVR (SEQ ID NO: 14) | 78.04 | 1092.5386 | 10 | 1.4 | 547.2773 |
| ALWELAETLVPGGK (SEQ ID NO: 192) | 77.03 | 1482.8082 | 14 | -0.1 | 742.4113 |
| YFGGSYENLNYNHK (SEQ ID NO: 11) | 76.42 | 1704.7532 | 14 | 1.1 | 853.3848 |
| VINRYFGGSYENLNYNHK (SEQ ID NO: 193) | 72.79 | 2187.0498 | 18 | 1.7 | 730.0251 |
| WINDY(+42.01)GGR (SEQ ID NO: 10) | 72.45 | 1033.5193 | 9 | 2.3 | 517.7681 |
| SK(+42.01)EK(+42.01)QEK(+42.01)ITDTFK (SEQ ID NO: 21) | 72.39 | 1706.8727 | 13 | 1.9 | 854.4453 |
| ALWELAETLVPGGK(+42.01)C(+57.02)R (SEQ ID NO: 194) | 72.38 | 1840.9506 | 16 | 0.7 | 921.4832 |
| Y(+42.01)KC(+57.02)FEDILK(+42.01)TPK(+42.01)SEIAK | 71.89 | 2195.1184 | 17 | 0.4 | 732.7137 |
| (SEQ ID NO: 195) | | | | | |
| NRK(+42.01)AILDLPGVGK(+42.01)K (SEQ ID NO: 164) | 71.71 | 1591.9409 | 14 | 2.6 | 531.6556 |
| AILDLPGVGK (SEQ ID NO: 196) | 71.39 | 981.5858 | 10 | 2.5 | 491.8014 |
| VFVSTILTFWNTDR (SEQ ID NO: 13) | 71.07 | 1697.8777 | 14 | 1 | 849.947 |
| TPK(+42.01)SEIAK(+42.01)DIK (SEQ ID NO: 50) | 70.81 | 1312.7238 | 11 | 2.3 | 657.3707 |
| TTAGHVK(+42.01)K(+42.01)IYDK (SEQ ID NO: 25) | 70.55 | 1443.7721 | 12 | 2.1 | 722.8948 |
| AAM(+15.99)VDANFVR (SEQ ID NO: 14) | 69.68 | 1108.5334 | 10 | 1.8 | 555.275 |
| K(+42.01)VDRLDDATNK (SEQ ID NO: 39) | 69.47 | 1315.6731 | 11 | 2.4 | 439.566 |
| TPK(+42.01)S(+42.01)EIAK(+42.01)DIK (SEQ ID NO: 50) | 69.14 | 1354.7344 | 11 | 2.3 | 678.376 |
| KVFVSTILTFWNTDR (SEQ ID NO: 9) | 68.57 | 1825.9727 | 15 | 0.5 | 609.6652 |
| NRK(+42.01)AILDLPGVGK (SEQ ID NO: 164) | 67.5 | 1421.8354 | 13 | -0.6 | 474.9521 |
| K(+42.01)AILDLPGVGK(+42.01)K (SEQ ID NO: 26) | 67.2 | 1321.7969 | 12 | 1.9 | 661.907 |
| VVINDYGGRVPR (SEQ ID NO: 59) | 67.04 | 1343.731 | 12 | 0 | 672.8727 |
| YK(+42.01)C(+57.02)FEDILK(+42.01)TPK (SEQ ID NO: 197) | 66.95 | 1624.817 | 12 | 6.5 | 813.4211 |
| VINRYFGGSYENLN(+.98)YNHKALWELAETLVPGGK (SEQ ID NO: 198) | 66.53 | 3652.8313 | 32 | 6.1 | 731.578 |
| IYDKFFVK(+42.01)YK (SEQ ID NO: 42) | 66.28 | 1391.7489 | 10 | −4.5 | 696.8786 |
| GH(+42.01)HHHHHSK(+42.01)K(+42.01)SGK (SEQ ID NO: 199) | 66.08 | 1638.7876 | 13 | -0.3 | 410.704 |
| KAAMVDANFVR (SEQ ID NO: 24) | 65.78 | 1220.6335 | 11 | 1 | 407.8855 |
| IYDK(+42.01)FFVK(+42.01)YK (SEQ ID NO: 42) | 65.67 | 1433.7594 | 10 | -0.4 | 717.8867 |
| KAILDLPGVGK(+42.01)K (SEQ ID NO: 26) | 65.61 | 1279.7864 | 12 | -0.7 | 427.6024 |
| EKQEK(+42.01)ITDTFK (SEQ ID NO: 30) | 65.54 | 1407.7245 | 11 | -0.6 | 704.8691 |
| S(+42.01)EIAK(+42.01)DIK(+42.01)EIGLSNQR (SEQ ID NO: 18) | 65.37 | 1926.0057 | 16 | -0.1 | 643.0091 |
| VDRLDDATNK (SEQ ID NO: 23) | 64.88 | 1145.5676 | 10 | 2.8 | 382.8642 |
| EK(+42.01)QEK(+42.01)ITDTFK(+42.01)VK (SEQ ID NO: 29) | 64.59 | 1718.9091 | 13 | 1 | 860.4626 |
| DPYVILIT(+42.01)SILLRR (SEQ ID NO: 19) | 64.58 | 1713.0188 | 14 | 2 | 572.0147 |
| YFGGSYENLN(+.98)YNHK (SEQ ID NO: 11) | 64.12 | 1705.7372 | 14 | 9.1 | 853.8836 |
| SKEK(+42.01)Q(+.98)EK(+42.01)ITDTFK (SEQ ID NO: 21) | 64.03 | 1665.8461 | 13 | 17.3 | 833.9447 |
| DFN(+.98)LGLM(+15.99)DFSAIIC(+57.02)APR (SEQ ID NO: 174) | 63.78 | 1955.9121 | 17 | 11.6 | 978.9747 |
| TC(+57.02)AAVM(+15.99)C(+57.02)LAFGK (SEQ ID NO: 200) | 63.78 | 1343.6036 | 12 | 3.7 | 672.8116 |
| YKC(+57.02)FEDILK(+42.01)TPK (SEQ ID NO: 197) | 63.76 | 1582.8065 | 12 | 1.7 | 792.4119 |
| NRK(+42.01)AILDLPGVGKK (SEQ ID NO: 188) | 63.75 | 1549.9303 | 14 | 1.3 | 517.6514 |
| YFGGSYENLNYN(+.98)HK (SEQ ID NO: 11) | 63.64 | 1705.7372 | 14 | 5.7 | 853.8807 |
| VINRYFGGSYENLN(+.98)YNHK (SEQ ID NO: 193) | 63.31 | 2188.0337 | 18 | 5.3 | 730.3557 |
| SEIAKDIKEIGLSNQR (SEQ ID NO: 18) | 63.26 | 1799.9741 | 16 | 0.6 | 600.999 |
| VINRYFGGSYEN(+.98)LNYNHK (SEQ ID NO: 193) | 63.2 | 2188.0337 | 18 | 9.2 | 730.3585 |
| SEIAK(+42.01)DIK(+42.01)EIGLS(+42.01)NQR (SEQ ID NO: 18) | 63.11 | 1926.0057 | 16 | -0.1 | 643.0091 |
| SKEKQEKITDTFK (SEQ ID NO: 21) | 63.09 | 1580.8409 | 13 | 3.4 | 527.9561 |
| SEIAKDIKEIGLSNQRAEQLK (SEQ ID NO: 204) | 62.9 | 2369.2913 | 21 | 1.8 | 593.3312 |
| KIYDK(+42.01)FFVK (SEQ ID NO: 46) | 62.44 | 1228.6855 | 9 | 1.3 | 410.5697 |
| K(+42.01)AAMVDANFVR (SEQ ID NO: 24) | 62.35 | 1262.6442 | 11 | 1.6 | 632.3304 |
| K(+42.01)AILDLPGVGK (SEQ ID NO: 26) | 62.32 | 1151.6914 | 11 | 0.7 | 576.8534 |
| K(+42.01)IYDKFFVK(+42.01)YK (SEQ ID NO: 205) | 62.27 | 1561.8544 | 11 | 6.9 | 781.9399 |
| S(+42.01)KEK(+42.01)QEK(+42.01)ITDTFK (SEQ ID NO: 21) | 61.92 | 1706.8727 | 13 | 1.2 | 569.9655 |
| K(+42.01)AAM(+15.99)VDANFVR (SEQ ID NO: 24) | 61.84 | 1278.639 | 11 | 3.4 | 640.329 |
| YFGGSY(+42.01)ENLNY(+42.01)NHK (SEQ ID NO: 11) | 61.81 | 1788.7743 | 14 | 1.6 | 895.3958 |
| KAAM(+15.99)VDANFVR (SEQ ID NO: 24) | 61.65 | 1236.6284 | 11 | 2 | 413.2176 |
| QEK(+42.01)ITDTFK(+42.01)VK (SEQ ID NO: 44) | 61.64 | 1419.7609 | 11 | 1.9 | 710.889 |
| VINRYFGGSYENLNY(+42.01)NHK (SEQ ID NO: 193) | 61.43 | 2229.0603 | 18 | 8.6 | 558.2772 |
| FFVK(+42.01)YK (SEQ ID NO: 62) | 61.2 | 872.4796 | 6 | -0.6 | 873.4863 |
| YKC(+57.02)FEDILK (SEQ ID NO: 201) | 61.19 | 1214.6005 | 9 | −1.2 | 405.8736 |
| VVIN(+.98)DYGGR (SEQ ID NO: 10) | 60.88 | 992.4927 | 9 | -0.3 | 497.2534 |
| YFGGS(+42.01)YENLNY(+42.01)NHK (SEQ ID NO: 11) | 60.85 | 1788.7743 | 14 | 3.4 | 597.2674 |
| Y(+42.01)FGGSYEN(+.98)LNY(+42.01)NHK (SEQ ID NO: 11) | 60.68 | 1789.7583 | 14 | 10.1 | 895.8954 |
| IYDK(+42.01)FFVK (SEQ ID NO: 55) | 60.5 | 1100.5906 | 8 | 2.3 | 551.3038 |
| KAILDLPGVGKK (SEQ ID NO: 206) | 60.49 | 1237.7758 | 12 | 0.5 | 413.5994 |
| K(+42.01)VDRLDDATNKK (SEQ ID NO: 41) | 60.39 | 1443.7681 | 12 | -0.6 | 361.9491 |
| LDDATNK(+42.01)KR (SEQ ID NO: 57) | 60.21 | 1101.5778 | 9 | −7.2 | 368.1972 |
| VFVSTILTFWNTDRR (SEQ ID NO: 31) | 60.2 | 1853.9788 | 15 | 1.4 | 619.001 |
| QEK(+42.01)ITDTFK (SEQ ID NO: 40) | 59.59 | 1150.5869 | 9 | 1.9 | 576.3018 |
| VVINDY(+42.01)GGRVPR (SEQ ID NO: 59) | 59.56 | 1385.7415 | 12 | 1.3 | 693.8789 |
| QEK(+42.01)ITDTFK(+42.01)VKR (SEQ ID NO: 37) | 59.55 | 1575.8621 | 12 | 0.8 | 788.939 |
| K(+42.01)AILDLPGVGKK (SEQ ID NO: 206) | 59.23 | 1279.7864 | 12 | 0.6 | 427.603 |
| QEK(+42.01)ITDTFK(+42.01)VK(+42.01)R (SEQ ID NO: 37) | 58.93 | 1617.8726 | 12 | 1.1 | 809.9445 |
| QEK(+42.01)ITDTFKVK (SEQ ID NO: 44) | 58.83 | 1377.7504 | 11 | -0.4 | 460.2572 |
| VSTILTFWNTDR (SEQ ID NO: 34) | 58.69 | 1451.7408 | 12 | 1.7 | 726.8789 |
| VIN(+.98)RYFGGSYENLNYNHK (SEQ ID NO: 193) | 58.63 | 2188.0337 | 18 | 8.8 | 730.3583 |
| AEQLKELAR (SEQ ID NO: 47) | 58.37 | 1056.5928 | 9 | 4.9 | 529.3063 |
| DIKEIGLSNQR (SEQ ID NO: 36) | 58.2 | 1271.6833 | 11 | -0.1 | 636.8489 |
| ITDTFK(+42.01)VK(+42.01)R (SEQ ID NO: 45) | 57.78 | 1190.6659 | 9 | 1.8 | 397.8966 |
| Y(+42.01)FGGSYENLNY(+42.01)NHK (SEQ ID NO: 11) | 57.4 | 1788.7743 | 14 | −2.2 | 597.264 |
| C(+57.02)FEDILK (SEQ ID NO: 202) | 57.26 | 923.4422 | 7 | 0.8 | 462.7288 |
| YFGGSYENLNYNHKALWELAETLVPGGK (SEQ ID NO: 7) | 57.23 | 3169.5508 | 28 | 5.2 | 793.3991 |
| TPK(+42.01)SEIAK (SEQ ID NO: 70) | 56.95 | 914.5073 | 8 | 0.3 | 458.261 |
| QEK(+42.01)ITDTFKVKR (SEQ ID NO: 37) | 56.78 | 1533.8514 | 12 | 2.5 | 512.2924 |
| IYDKFFVK (SEQ ID NO: 55) | 56.76 | 1058.5801 | 8 | 2.5 | 530.2986 |
| LDDATNK (SEQ ID NO: 58) | 56.68 | 775.3712 | 7 | -0.5 | 776.3781 |
| DIK(+42.01)EIGLSNQR (SEQ ID NO: 36) | 56.51 | 1313.6938 | 11 | 2.3 | 657.8557 |
| LDDATNKKR (SEQ ID NO: 57) | 56.27 | 1059.5673 | 9 | 0.3 | 530.7911 |
| KAILDLPGVGK (SEQ ID NO: 26) | 56.14 | 1109.6808 | 11 | 2.4 | 555.849 |
| EIGLSNQRAEQLKELAR (SEQ ID NO: 207) | 55.83 | 1954.0596 | 17 | 0.8 | 489.5226 |
| ITDTFKVK(+42.01)R (SEQ ID NO: 45) | 55.49 | 1148.6553 | 9 | 0.2 | 383.8924 |
| VDRLDDATNK(+42.01)K(+42.01)R (SEQ ID NO: 121) | 55.33 | 1513.7848 | 12 | 2.7 | 505.6035 |
| EK(+42.01)QEK(+42.01)ITDTFK (SEQ ID NO: 30) | 55.24 | 1449.7351 | 11 | 0.4 | 725.8751 |
| LDDATNKK(+42.01)R (SEQ ID NO: 57) | 54.84 | 1101.5778 | 9 | −1.8 | 368.1992 |
| KPK(+42.01)C(+57.02)EK(+42.01)C(+57.02)GMSK | 54.8 | 1435.6621 | 11 | 1.2 | 718.8392 |
| (SEQ ID NO: 114) | | | | | |
| EKQ(+.98)EK(+42.01)ITDTFK(+42.01)VK (SEQ ID NO: 29) | 54.74 | 1677.8824 | 13 | 10.5 | 839.9573 |
| ITDTFK(+42.01)VKR (SEQ ID NO: 45) | 54.4 | 1148.6553 | 9 | 2.8 | 383.8934 |
| AILDLPGVGKK (SEQ ID NO: 208) | 54.33 | 1109.6808 | 11 | -0.1 | 370.9008 |
| KVFVSTILTFWN(+.98)TDR (SEQ ID NO: 9) | 54.05 | 1826.9567 | 15 | 11.5 | 914.4962 |
| C(+57.02)EK(+42.01)C(+57.02)GMSK (SEQ ID NO: 209) | 53.97 | 1040.4089 | 8 | 1.9 | 521.2127 |
| ITDTFKVKR (SEQ ID NO: 45) | 53.83 | 1106.6448 | 9 | 3.6 | 554.3317 |
| K(+42.01)SGK(+42.01)SAK(+42.01)SK (SEQ ID NO: 88) | 53.47 | 1045.5768 | 9 | −2.6 | 1046.5813 |
| KVDRLDDATNK (SEQ ID NO: 39) | 53.12 | 1273.6626 | 11 | 1.3 | 637.8394 |
| SAK(+42.01)SK(+42.01)EK(+42.01)QEK (SEQ ID NO: 63) | 52.91 | 1287.667 | 10 | 1.1 | 644.8415 |
| LC(+57.02)SYYEK (SEQ ID NO: 82) | 52.81 | 961.4215 | 7 | −1 | 481.7175 |
| SGK(+42.01)SAK(+42.01)SK(+42.01)EK (SEQ ID NO: 76) | 52.75 | 1174.6194 | 10 | 0.6 | 588.3173 |
| DFPWR (SEQ ID NO: 90) | 52.67 | 719.3391 | 5 | 0.6 | 360.677 |
| K(+42.01)IY(+42.01)DK(+42.01)FFVK (SEQ ID NO: 46) | 52.51 | 1312.7067 | 9 | 5.7 | 657.3644 |
| KVDRLDDATNKK (SEQ ID NO: 41) | 52.46 | 1401.7576 | 12 | 0.1 | 351.4467 |
| TDTFK (SEQ ID NO: 112) | 52.46 | 610.2962 | 5 | 1.1 | 611.3041 |
| K(+42.01)IYDKFFVK (SEQ ID NO: 46) | 51.95 | 1228.6855 | 9 | 3.5 | 615.3522 |
| K(+42.01)IYDK(+42.01)FFVK (SEQ ID NO: 46) | 51.5 | 1270.696 | 9 | -0.3 | 636.3551 |
| YFGGSYEN(+.98)LN(+.98)YNHK (SEQ ID NO: 11) | 51.19 | 1706.7212 | 14 | 11.6 | 854.3778 |
| AEQLK(+42.01)ELAR (SEQ ID NO: 47) | 51.08 | 1098.6033 | 9 | 2.2 | 550.3101 |
| SEIAKDIK(+42.01)EIGLSN(+.98)QR (SEQ ID NO: 18) | 51.03 | 1842.9686 | 16 | 13.6 | 922.5042 |
| ITDTFK (SEQ ID NO: 65) | 50.84 | 723.3803 | 6 | 0 | 724.3876 |
| IYDKFFVKYK (SEQ ID NO: 42) | 50.66 | 1349.7383 | 10 | 4.2 | 450.9219 |
| LDDATNK(+42.01)K(+42.01)R (SEQ ID NO: 57) | 50.58 | 1143.5884 | 9 | 0.8 | 572.8019 |
| DIK(+42.01)EIGLS(+42.01)NQR (SEQ ID NO: 36) | 50.49 | 1355.7045 | 11 | 0.4 | 678.8598 |
| GH(+42.01)HHHHHSK(+42.01)K (SEQ ID NO: 210) | 50.48 | 1324.6285 | 10 | 0.8 | 442.5505 |
| TPK(+42.01)S(+42.01)EIAK (SEQ ID NO: 70) | 50.4 | 956.5178 | 8 | 3 | 479.2676 |
| GH(+42.01)HHHHHS(+42.01)KK(+42.01)SGK (SEQ ID NO: 199) | 50.27 | 1638.7876 | 13 | −1 | 410.7038 |
| EIGLSNQR (SEQ ID NO80) | 50.11 | 915.4774 | 8 | 0.8 | 458.7463 |
| ITDTFK(+42.01)VK(+42.01)RK (SEQ ID NO: 211) | 49.99 | 1318.7609 | 10 | -0.1 | 660.3876 |
| IY(+42.01)DK(+42.01)FFVK (SEQ ID NO: 55) | 49.51 | 1142.6012 | 8 | 3.2 | 572.3097 |
| TTAGHVK(+42.01)K (SEQ ID NO: 71) | 49.37 | 882.4923 | 8 | -0.2 | 442.2534 |
| ILTFWNTDR (SEQ ID NO: 52) | 49.18 | 1164.5928 | 9 | 1.9 | 583.3047 |
| VINRYFGGSYEN(+.98)LNYN (SEQ ID NO: 203) | 49.18 | 1922.8799 | 16 | 8.2 | 962.4551 |
| TTAGHVK(+42.01) (SEQ ID NO: 84) | 48.92 | 754.3973 | 7 | 0 | 378.206 |
| DIK(+42.01)EIGLSNQRAEQLKELAR (SEQ ID NO: 212) | 48.74 | 2352.2761 | 20 | 6.9 | 589.0804 |
| EK(+42.01)QEKITDTFK (SEQ ID NO: 30) | 48.71 | 1407.7245 | 11 | 1.4 | 470.2494 |
| YK(+42.01)C(+57.02)FEDILK (SEQ ID NO: 201) | 48.66 | 1256.6111 | 9 | -0.9 | 629.3123 |
| KIYDKFFVK (SEQ ID NO: 46) | 48.45 | 1186.6749 | 9 | 1.9 | 396.5663 |
| QEKITDTFK (SEQ ID NO: 40) | 47.96 | 1108.5764 | 9 | 3.3 | 370.534 |
| SEIAK(+42.01)DIK (SEQ ID NO: 67) | 47.94 | 944.5178 | 8 | 1.1 | 473.2667 |
| RKVDRLDDATNK (SEQ ID NO: 213) | 47.47 | 1429.7637 | 12 | -0.4 | 358.4481 |
| GH(+42.01)HHHHHSK(+42.01) (SEQ ID NO: 214) | 47.35 | 1196.5336 | 9 | 0.4 | 399.852 |
| KVFVSTILTFWNTDRR (SEQ ID NO: 28) | 47.28 | 1982.0737 | 16 | 0.9 | 661.6991 |
| LC(+57.02)SYYEK(+42.01)C(+57.02)ST (SEQ ID NO: 215) | 46.89 | 1351.5425 | 10 | −2.4 | 676.7769 |
| C(+57.02)EK(+42.01)C(+57.02)GM(+15.99)SK (SEQ ID NO:) | 46.84 | 1056.4038 | 8 | -0.3 | 529.209 |
| YDKFFVK (SEQ ID NO: 216) | 46.76 | 945.496 | 7 | 2.2 | 473.7563 |
| SEIAKDIK (SEQ ID NO: 67) | 46.5 | 902.5073 | 8 | 2.1 | 903.5164 |
| SGK(+42.01)SAK(+42.01)SK (SEQ ID NO: 89) | 46.44 | 875.4712 | 8 | 1.3 | 438.7434 |
| LDDATNK(+42.01)K (SEQ ID NO: 54) | 46.3 | 945.4767 | 8 | 1.9 | 473.7465 |
| TTAGHVK (SEQ ID NO: 84) | 46.11 | 712.3868 | 7 | 0.5 | 357.2008 |
| LDDATNK(+42.01)K(+42.01)RK (SEQ ID NO: 217) | 45.97 | 1271.6833 | 10 | 1.7 | 636.85 |
| EK(+42.01)Q(+.98)EK(+42.01)ITDTFK (SEQ ID NO: 30) | 45.9 | 1450.7191 | 11 | 13 | 726.3763 |
| S(+42.01)EIAK(+42.01)DIK (SEQ ID NO: 67) | 45.65 | 986.5284 | 8 | 8.2 | 987.5438 |
| VDRLDDATNKK(+42.01)R (SEQ ID NO: 218) | 45.61 | 1471.7743 | 12 | 0.8 | 491.5991 |
| SEIAK(+42.01)DIK(+42.01)EIGLSN(+.98)QR (SEQ ID NO: 18) | 45.45 | 1884.9792 | 16 | 10.5 | 943.5068 |
| LC(+57.02)SYY(+42.01)EK (SEQ ID NO: 82) | 45.31 | 1003.4321 | 7 | 0.8 | 502.7237 |
| LC(+42.01)SYYEK (SEQ ID NO: 82) | 45.28 | 946.4106 | 7 | −2.5 | 474.2114 |
| SAK(+42.01)SK(+42.01)EK (SEQ ID NO: 108) | 45.25 | 860.4603 | 7 | -0.2 | 431.2374 |
| EIGLS(+42.01)NQR (SEQ ID NO: 48) | 45.22 | 957.4879 | 8 | −2.1 | 479.7502 |
| RTTAGHVK(+42.01)K(+42.01)IYDK (SEQ ID NO: 219) | 45.2 | 1599.8733 | 13 | −2.2 | 800.9421 |
| T(+42.01)TAGHVK (SEQ ID NO: 84) | 45.19 | 754.3973 | 7 | −2.1 | 378.2052 |
| Y(+42.01)FGGSYEN(+.98)LNYNHK (SEQ ID NO: 11) | 45.06 | 1747.7478 | 14 | 11.6 | 583.5966 |
| VINRYFGGSYENLN (SEQ ID NO: 220) | 45.02 | 1644.7896 | 14 | −1.6 | 823.4008 |
| EIGLSN(+.98)QR (SEQ ID NO: 48) | 44.89 | 916.4614 | 8 | 5.7 | 459.2406 |
| TLVPGGK (SEQ ID NO: 103) | 44.81 | 670.4014 | 7 | 0.3 | 671.4088 |
| ITDTFKVK (SEQ ID NO: 64) | 44.56 | 950.5436 | 8 | 2.3 | 476.2802 |
| GH(+42.01)HHHHHS(+42.01)KK (SEQ ID NO: 210) | 44.48 | 1324.6285 | 10 | 4.3 | 442.552 |
| RDFPWR (SEQ ID NO: 68) | 44.47 | 875.4402 | 6 | 1.8 | 438.7281 |
| T(+42.01)TAGHVK(+42.01)K (SEQ ID NO: 84) | 44.26 | 924.5029 | 8 | 3.3 | 463.2602 |
| TC(+57.02)AAVM(+15.99)C(+57.02)LAFGK(+42.01)K | 44.21 | 1513.7091 | 13 | 5.9 | 757.8663 |
| (SEQ ID NO: 221) | | | | | |
| ITDTFK(+42.01)VK (SEQ ID NO: 64) | 43.94 | 992.5542 | 8 | 2.6 | 497.2857 |
| K(+42.01)PK(+42.01)C(+57.02)EK (SEQ ID NO: 222) | 43.87 | 872.4426 | 6 | −2.1 | 873.448 |
| WIND (SEQ ID NO: 223) | 43.7 | 558.3013 | 5 | 2.7 | 559.3101 |
| LC(+57.02)S(+42.01)YYEK (SEQ ID NO: 82) | 43.62 | 1003.4321 | 7 | 1.1 | 502.7239 |
| AEQLK (SEQ ID NO: 93) | 42.99 | 587.3279 | 5 | 0.3 | 588.3353 |
| VINRYFGGSYENLNYN (SEQ ID NO: 203) | 42.92 | 1921.8959 | 16 | −1.9 | 641.638 |
| KSGK(+42.01)SAK(+42.01)SK (SEQ ID NO: 88) | 42.86 | 1003.5662 | 9 | −1 | 502.7899 |
| IT(+42.01)DTFK (SEQ ID NO: 65) | 42.23 | 765.3909 | 6 | 1.1 | 383.7031 |
| VINRYFGGSYEN(+.98)LN(+.98)YNHK (SEQ ID NO: 193) | 41.86 | 2189.0178 | 18 | 14.1 | 548.2695 |
| N(+.98)RK(+42.01)AILDLPGVGKK (SEQ ID NO: 188) | 41.8 | 1550.9143 | 14 | 13.5 | 388.7411 |
| EK(+42.01)QEKITDTFKVK (SEQ ID NO: 29) | 41.38 | 1634.8879 | 13 | -0.7 | 545.9695 |
| AILDLPGVGK(+42.01)K (SEQ ID NO: 208) | 40.91 | 1151.6914 | 11 | 1.7 | 576.8539 |
| KVDRLDDATNK(+42.01)K (SEQ ID NO: 41) | 40.58 | 1443.7681 | 12 | 1.7 | 482.2641 |
| FFVK(+42.01)Y(+42.01)K (SEQ ID NO: 62) | 40.18 | 914.4902 | 6 | -0.3 | 458.2522 |
| S(+42.01)GK(+42.01)SAK (SEQ ID NO: 126) | 40.11 | 660.3442 | 6 | 0 | 661.3515 |
| LC(+57.02)SY(+42.01)YEK (SEQ ID NO: 82) | 40.01 | 1003.4321 | 7 | -0.6 | 502.723 |
| TAGHVK(+42.01)K (SEQ ID NO: 138) | 39.82 | 781.4446 | 7 | -0.3 | 391.7295 |
| VINRYFGGSYEN (SEQ ID NO: 224) | 39.68 | 1417.6626 | 12 | −1.6 | 709.8375 |
| YFGGSYEN(+.98)LNYNHK (SEQ ID NO: 11) | 39.67 | 1705.7372 | 14 | 13.7 | 569.5941 |
| VFVSTILTFWN(+.98)TDR (SEQ ID NO: 13) | 39.04 | 1698.8617 | 14 | 17.5 | 850.453 |
| YFGGSYENLNYN (SEQ ID NO: 225) | 38.95 | 1439.5994 | 12 | 4.1 | 720.8099 |
| SEIAKDIKEIGLSN(+.98)OR (SEQ ID NO: 18) | 38.7 | 1800.9581 | 16 | 12.1 | 901.4973 |
| LDLPGVGKK (SEQ ID NO: 226) | 38.66 | 925.5596 | 9 | 2.1 | 463.7881 |
| EK(+42.01)Q(+.98)EKITDTFK (SEQ ID NO: 30) | 38.33 | 1408.7085 | 11 | 13.3 | 470.583 |
| AAMVDAN(+.98)FVR (SEQ ID NO: 14) | 38.26 | 1093.5226 | 10 | 18 | 365.5214 |
| RTTAGHVK(+42.01) (SEQ ID NO: 227) | 37.43 | 910.4985 | 8 | 3 | 456.2579 |
| SEIAK (SEQ ID NO: 99) | 37.41 | 546.3013 | 5 | 1.2 | 547.3092 |
| YFGGSYEN(+.98)LNY(+42.01)NHK (SEQ ID NO: 11) | 37.24 | 1747.7478 | 14 | 12 | 583.5969 |
| TFWNTDR (SEQ ID NO: 72) | 37.2 | 938.4246 | 7 | 1.6 | 470.2203 |
| EIGLSN (SEQIDNO: 81) | 37.16 | 631.3177 | 6 | 0.5 | 632.3253 |
| ITS(+42.01)ILLR (SEQ ID NO: 228) | 36.87 | 856.5382 | 7 | 0.1 | 429.2764 |
| IK(+42.01)EIGLSNQR (SEQ ID NO: 83) | 36.8 | 1198.667 | 10 | 5.5 | 600.3441 |
| SGK(+42.01)S(+42.01)AK(+42.01)SK (SEQ ID NO: 89) | 36.78 | 917.4818 | 8 | −1.2 | 459.7476 |
| AAMVDANFVRVINRYFGGSYENLNYNHK (SEQ ID NO: 229) | 36.76 | 3261.5776 | 28 | −2 | 653.3215 |
| K(+42.01) S(+42.01)GK(+42.01)SAK (SEQ ID NO: 113) | 36.45 | 830.4498 | 7 | −1.9 | 831.4555 |
| WELAETLVPGGK (SEQ ID NO: 38) | 36.44 | 1298.687 | 12 | 1.7 | 650.3519 |
| S(+42.01)EIAK (SEQ ID NO: 99) | 36.35 | 588.3119 | 5 | -0.4 | 589.3189 |
| FED ILK (SEQ ID NO: 111) | 35.91 | 763.4116 | 6 | 2.3 | 382.7139 |
| INDYGGR (SEQ ID NO: 132) | 35.73 | 793.3718 | 7 | −5 | 397.6912 |
| K(+42.01)SGK(+42.01)SAK (SEQ ID NO: 113) | 35.68 | 788.4392 | 7 | 0.3 | 395.227 |
| AAMVDAN (SEQ ID NO: 101) | 35.53 | 690.3007 | 7 | −5.8 | 691.304 |
| S(+42.01)KEK(+42.01)QEK (SEQ ID NO: 109) | 34.96 | 959.4923 | 7 | 1.3 | 480.7541 |
| S(+42.01)GK(+42.01)SAK(+42.01)SK (SEQ ID NO: 230) | 34.39 | 917.4818 | 8 | 2 | 459.7491 |
| AAM(+15.99)VDANFVRVINRYFGGSYENLNYNHK (SEQ ID NO: 229) | 33.26 | 3277.5728 | 28 | -0.1 | 656.5217 |
| VFVSTILT (SEQ ID NO: 231) | 33.23 | 878.5113 | 8 | 1.6 | 879.52 |
| NY(+42.01)NH(+42.01)KALWELAETLVPGGK (SEQ ID NO: 232) | 33.08 | 2223.1323 | 19 | −1.6 | 742.0502 |
| YFGGSYEN(+,98)LNYNH (SEQ ID NO: 27) | 33.03 | 1577.6422 | 13 | 13.5 | 789.8391 |
| NYN(+.98)H(+42.01)K(+42.01)ALWELAETLVPGGK | 32.92 | 2224.1165 | 19 | 1.4 | 742.3805 |
| (SEQ ID NO: 232) | | | | | |
| K(+42.01)IYDK (SEQ ID NO: 123) | 32.89 | 707.3854 | 5 | 0.3 | 708.3929 |
| C(+57.02)GM(+15.99)S(+42.01)KLC(+42.01)SYYEK | 32.73 | 1567.6357 | 12 | 7.5 | 784.831 |
| (SEQ ID NO: 233) | | | | | |
| VM(+15.99)C(+57.02)LAFGKK(+42.01)AAMVDANFVR | 32.7 | 2185.0845 | 19 | −5.1 | 547.2756 |
| (SEQ ID NO: 234) | | | | | |
| RTTAGHVK(+42.01)K (SEQ ID NO: 134) | 32.36 | 1038.5934 | 9 | -0.6 | 520.3036 |
| TTAGHVK(+42.01)K(+42.01)IYDK(+42.01)F (SEQ ID NO: 80) | 32.17 | 1632.8511 | 13 | 1.4 | 545.2917 |
| VINDYGGR (SEQ ID NO: 125) | 32.12 | 892.4402 | 8 | −1.2 | 447.2269 |
| DIK(+42.01)EIGLSN(+.98)QR (SEQ ID NO: 36) | 32.07 | 1314.6779 | 11 | 15 | 439.2398 |
| K(+42.01)VDRLDDATN(+.98)K(+42.01)K (SEQ ID NO: 41) | 31.67 | 1486.7627 | 12 | 14.7 | 744.3995 |
| LDLPGVGK (SEQ ID NO: 117) | 31.58 | 797.4647 | 8 | 1.5 | 798.4731 |
| DATNK (SEQ ID NO: 137) | 30.91 | 547.2602 | 5 | -0.8 | 548.267 |
| DKFFVK (SEQ ID NO: 235) | 30.72 | 782.4326 | 6 | 1.1 | 392.224 |
| GH(+42.01)HHHHHS(+42.01)KK(+42.01) (SEQ ID NO: 210) | 30.42 | 1366.6392 | 10 | −2.1 | 456.5527 |
| SYYEK (SEQ ID NO: 236) | 30.26 | 688.3068 | 5 | 3.1 | 689.3162 |
| N(+.98)RK(+42.01)AILDLPGVGK (SEQ ID NO: 164) | 29.84 | 1422.8195 | 13 | 15.4 | 712.428 |
| C(+57.02)AAVM(+15.99)C(+42.01)LAFGK(+42.01)K | 29.39 | 1397.6505 | 12 | −2.6 | 699.8307 |
| (SEQ ID NO: 237) | | | | | |
| VFVSTIL (SEQ ID NO: 118) | 29.23 | 777.4636 | 7 | 0.7 | 778.4714 |
| Q(+.98)EK(+42.01)ITDTFK(+42.01)VK (SEQ ID NO: 44) | 29.17 | 1420.7449 | 11 | 14.2 | 711.3898 |
| DDATNK (SEQ ID NO: 167) | 28.91 | 662.2871 | 6 | -0.1 | 663.2943 |
| DDATNKKR (SEQ ID NO: 238) | 28.85 | 946.4832 | 8 | 1 | 474.2493 |
| DLPGVGKK (SEQ ID NO: 239) | 28.66 | 812.4756 | 8 | 3 | 407.2463 |
| N(+.98)YNH(+42.01)K(+42.01)ALWELAETLVPGGK | 28.65 | 2224.1165 | 19 | 12.6 | 742.3888 |
| (SEQ ID NO: 232) | | | | | |
| K(+42.01)AILDLPGVGK(+42.01)K(+42.01)T (SEQ ID NO: 240) | 28.62 | 1464.8551 | 13 | -0.2 | 733.4347 |
| VK(+42.01)K(+42.01)IYDK (SEQ ID NO: 133) | 28.6 | 976.5593 | 7 | 0.7 | 489.2873 |
| TDTFK(+42.01)VK (SEQ ID NO: 241) | 27.44 | 879.4702 | 7 | −3.8 | 880.4741 |
| ILDLPGVGKK (SEQ ID NO: 242) | 27.33 | 1038.6437 | 10 | 5.4 | 520.3319 |
| ALWELAETLVPGGK(+42.01) (SEQ ID NO: 15) | 27.32 | 1524.8187 | 14 | 14.4 | 763.4276 |
| YFGGSY(+42.01)EN(+.98)LNY(+42.01)NHK (SEQ ID NO: 11) | 27.19 | 1789.7583 | 14 | 12 | 597.6005 |
| C(+57.02)AAVMC(+42.01)LAFGK(+42.01)K (SEQ ID NO: 237) | 27.06 | 1381.6556 | 12 | 1.9 | 691.8364 |
| S(+42.01)K(+42.01)EK(+42.01)QEK (SEQ ID NO: 109) | 27.01 | 1001.5029 | 7 | 0.6 | 501.7591 |
| VVIN(+.98)DYGGRVPR (SEQ ID NO: 59) | 27.01 | 1344.715 | 12 | 15.7 | 449.2526 |
| GGSYEN(+.98)LNYNHK (SEQ ID NO: 243) | 26.94 | 1395.6055 | 12 | 8.6 | 698.816 |
| DFPWRHTR (SEQ ID NO: 244) | 26.41 | 1113.5468 | 8 | 1.3 | 557.7814 |
| C(+57.02)AAVM(+15.99)C(+42.01)LAFGK (SEQ ID NO: 245) | 25.9 | 1227.545 | 11 | 2 | 614.781 |
| EQLK(+42.01)ELAR (SEQ ID NO: 169) | 25.53 | 1027.5662 | 8 | 3.2 | 514.792 |
| EKQEKITDTFK (SEQ ID NO: 30) | 25.08 | 1365.714 | 11 | −19.6 | 456.2364 |
| YFGGSY(+42.01)ENLNYNHK (SEQ ID NO: 11) | 24.75 | 1746.7638 | 14 | −1.3 | 583.2611 |
| AAMVDANFVRVIN(+.98)RYFGGSYENLNYNHK (SEQ ID NO: 229) | 24.57 | 3262.5618 | 28 | 4.2 | 653.5223 |
| DFNLGLMDF (SEQ ID NO: 74) | 24.35 | 1070.4742 | 9 | 3.3 | 536.2462 |
| K(+42.01)VDRLDDATN(+.98)KK (SEQ ID NO: 41) | 23.52 | 1444.7521 | 12 | 10.5 | 723.3909 |
| RLDDATNK (SEQ ID NO: 246) | 23.23 | 931.4723 | 8 | −3.6 | 466.7417 |
| SEIAK(+42.01)DIKEIGLSNQ(+.98)R (SEQ ID NO: 18) | 23.07 | 1842.9686 | 16 | 12.5 | 615.3378 |
| VILITS(+42.01)ILLR (SEQ ID NO: 78) | 22.81 | 1181.7747 | 10 | −3.7 | 591.8924 |
| EIGLSNQ(+.98)RA (SEQ ID NO: 140) | 22.11 | 987.4985 | 9 | 0 | 494.7565 |
| C(+57.02)AAVMC(+42.01)LAFGK (SEQ ID NO: 245) | 21.57 | 1211.55 | 11 | 1.2 | 606.783 |
| QLKELAR (SEQ ID NO: 247) | 21.41 | 856.513 | 7 | 2 | 429.2646 |
| DLPGVGK (SEQ ID NO: 107) | 21.37 | 684.3806 | 7 | 1.3 | 685.3888 |
| DYGGR (SEQ ID NO: 160) | 21.14 | 566.2449 | 5 | 5.8 | 567.2554 |
| LSNQR (SEQ ID NO: 248) | 20.97 | 616.3293 | 5 | −2.6 | 617.335 |
| SAK(+42.01)SK (SEQ ID NO: 249) | 20.81 | 561.3122 | 5 | -0.1 | 562.3194 |
| KVFVSTILTFWN(+.98)TDRR (SEQ ID NO: 28) | 20.52 | 1983.0577 | 16 | 9.6 | 662.0329 |
| DFNLGL (SEQ ID NO: 115) | 20.46 | 677.3384 | 6 | −4.7 | 678.3425 |
| LDDAT(+42.01)NKK(+42.01)R (SEQ ID NO: 57) | 20.39 | 1143.5884 | 9 | −4.2 | 572.7991 |
| RK(+42.01)VDR (SEQ ID NO: 144) | 20.27 | 714.4136 | 5 | -0.5 | 358.2139 |
| LDDATNK(+42.01)KRK (SEQ ID NO: 217) | 20.26 | 1229.6727 | 10 | -0.5 | 615.8433 |
| KSGK(+42.01)S(+42.01)AKS(+42.01)K (SEQ ID NO: 88) | 20.12 | 1045.5768 | 9 | 3.8 | 523.7977 |
| TDTFKVK (SEQ ID NO: 241) | 18.43 | 837.4596 | 7 | 3.4 | 419.7385 |
| SK(+42.01)K(+42.01)SGK (SEQ ID NO: 250) | 18.21 | 717.4021 | 6 | -0.3 | 359.7082 |
| AAMVDAN(+.98)FVRVIN(+.98)RYFGGSYENLNYNHK | 17.89 | 3263.5457 | 28 | 5.4 | 816.8981 |
| (SEQ ID NO: 229) | | | | | |
| LVPGGK (SEQ ID NO: 147) | 17.65 | 569.3536 | 6 | 1.4 | 570.3617 |
| C(+57.02)AAVMC(+42.01)LAFGKK (SEQ ID NO: 237) | 17.43 | 1339.645 | 12 | -0.1 | 447.5556 |
| HTRDPYVILIT (SEQ ID NO: 251) | 17.41 | 1326.7296 | 11 | 8.4 | 1327.748 |
| AGHVK (SEQ ID NO: 252) | 17.15 | 510.2914 | 5 | 0.5 | 511.2989 |
| S(+42.01)KLC(+42.01)SYYEK (SEQ ID NO: 253) | 16.63 | 1203.5481 | 9 | −10.2 | 602.7752 |
| LS(+42.01)NQR (SEQ ID NO: 248) | 15.88 | 658.3398 | 5 | 1.9 | 659.3484 |
| LK(+42.01)ELAR (SEQ ID NO: 254) | 15.46 | 770.465 | 6 | -0.6 | 771.4718 |
| GHVK(+42.01)K (SEQ ID NO: 255) | 15.38 | 609.3598 | 5 | −3.6 | 610.3649 |
| DDATNK(+42.01)KR (SEQ ID NO: 287) | 15.18 | 988.4938 | 8 | 1.8 | 495.2551 |
| MS(+42.01)K(+42.01)LC(+42.01)SYYEK (SEQ ID NO: 189) | 15.13 | 1376.5992 | 10 | 13.1 | 689.3159 |

Lyases and endonucleases can cleave abasic sites on the 5′-side or the 3′-side of the abasic site (FIG. 20). To investigate the mechanism of cleavage by the hyTDG-lyase, a 5′-FAM labelled duplex containing a U:G mispair was incubated with the lyase at 65 TC for 1 h and the resulting oligonucleotide cleavage products were examined by MALDI-Tof-Tof-MS (FIG. 21). Lyase cleavage results in two oligonucleotide fragments. The oligonucleotide fragment arising from the 3′-end of the substrate had an observed m/z of 3446.58, consistent with a 5′-phosphate (FIG. 21B). The 5′-fragment with the 5′-FAM label had an observed m/z of 2601.2450, which is 60 mass units higher than the α-β-unsaturated sugar fragment seen for endo III cleavage. The water and β-mercaptoethanol present in the hyTDG-lyase purification protocol was contemplated to reacted with the abasic site aldehyde to generate the product shown in FIG. 21A. Recently, Gates and coworkers (26) presented evidence that β-mercaptoethanol could form an adduct with an abasic site, in accord with the data presented here. In FIG. 21A, a structure consistent with the observed mass is presented, although other structural isomers are possible.

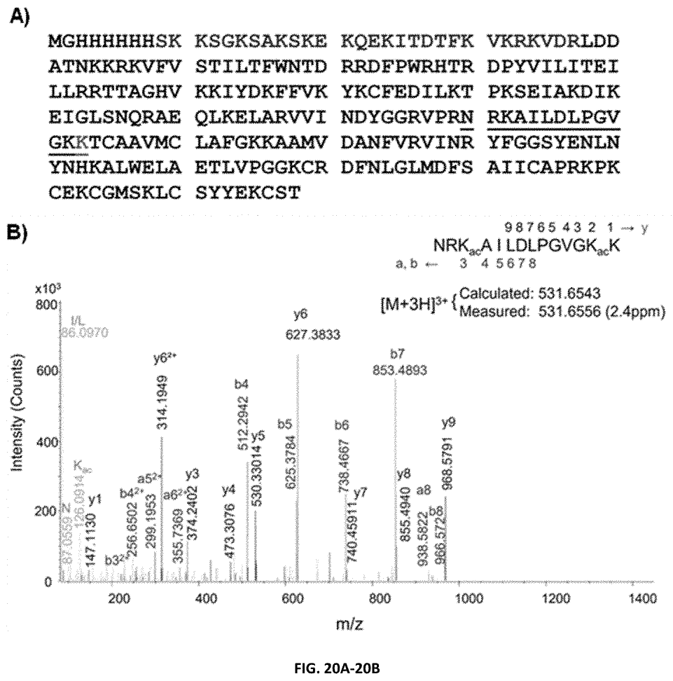

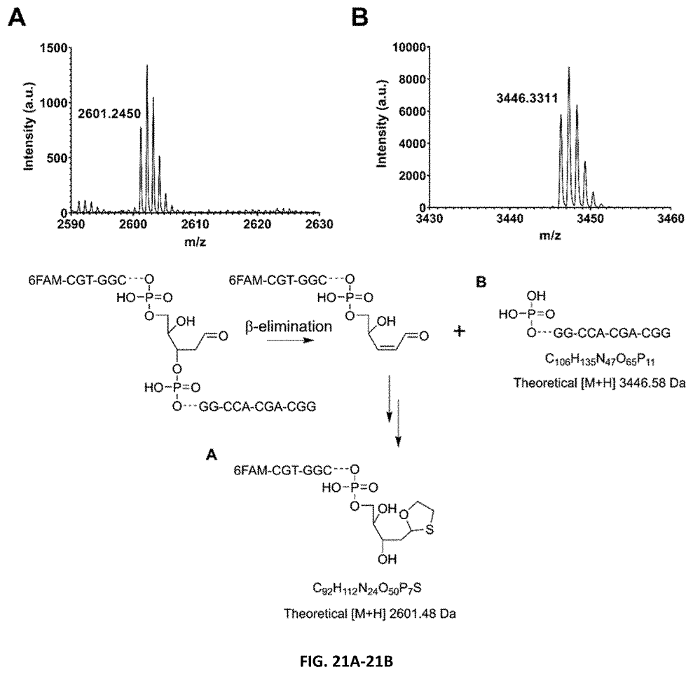

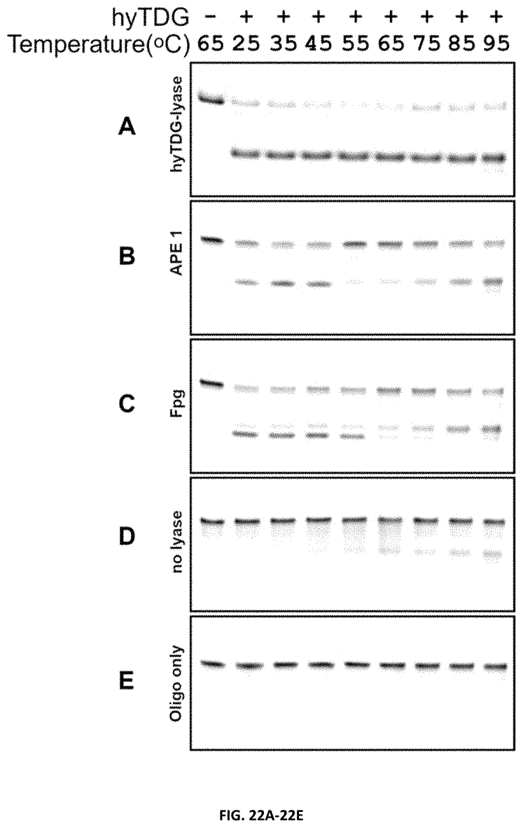

To test the lyase activity of the mutant protein, an oligonucleotide duplex was constructed containing a T:G mispair and a 5′-FAM label. This duplex was incubated with hyTDG at 65° C. for 1 h to generate an abasic site. The hyTDG-lyase was then added and the reaction mixture incubated at defined temperatures from 25° C. to 95° C. Substrate oligonucleotides were resolved by gel electrophoresis and imaged with a Storm imager (FIG. 22A). The hyTDG-lyase effectively cleaved the abasic-site containing oligonucleotide from 25° C. to 95° C.

To compare the activity of our hyTDG-lyase, abasic site-containing duplexes were also incubated with apurinic/apyrimidinic (abasic) endonuclease 1 (APE 1) (FIG. 22B) and formamidopyrimidine DNA glycosylase (Fpg) (FIG. 22C). APE 1 cleaves from 25° C. to 45° C. generating a 3′-OH on the 5′-side of the abasic site (FIG. 19). At higher temperatures above 45° C., cleavage is observed due to nonenzymatic β-elimination. Fpg cleaves the abasic site-containing oligonucleotide from 25° C. to 55° C. Spontaneous β-elimination is seen at higher temperatures as with APE 1. Spontaneous β-elimination of abasic site-containing oligonucleotides is observed at temperatures above 55° C. (FIG. 22D) in the absence of any lyase or endonuclease. Intact oligonucleotides containing no abasic sites do not spontaneously cleave under these conditions (FIG. 22E).

Next, the inventors examined the activity of the hyTDG-lyase in various buffer systems (FIG. 23A). Using an oligonucleotide duplex containing a U:G mispair, the U was cleaved with uracil DNA-glycosylase (UDG) to generate an abasic site. In TDG buffer, the abasic site can be cleaved by addition of NaOH, or by the lyases hyTDG-lyase and APE 1. In contrast, in UDG buffer, the abasic site-containing oligonucleotide is cleaved by NaOH as well as the hyTDG-lyase, but not by APE 1.

While the data shown in FIG. 23A indicates that abasic sites are effectively cleaved by added NaOH, some modified bases are degraded by NaOH (FIG. 23B). An oligonucleotide duplex containing 5-formylcytosine (5foC) opposite to G incubated with hTDG, followed by NaOH, hyTDG-lyase or APE 1 treatment (FIG. 23B). Incubation of the 5foC-containing oligonucleotide with NaOH results in both base hydrolysis and β-elimination. Incubation with hTDG in either TDG or UDG buffers followed by hyTDG-lyase resulted in cleavage of approximately half of the 5foC-containing oligonucleotides. Incubation of the 5foC-containing oligonucleotide with hTDG followed by APE 1 in TDG buffer, but not in UDG buffer resulted in predominant cleavage.

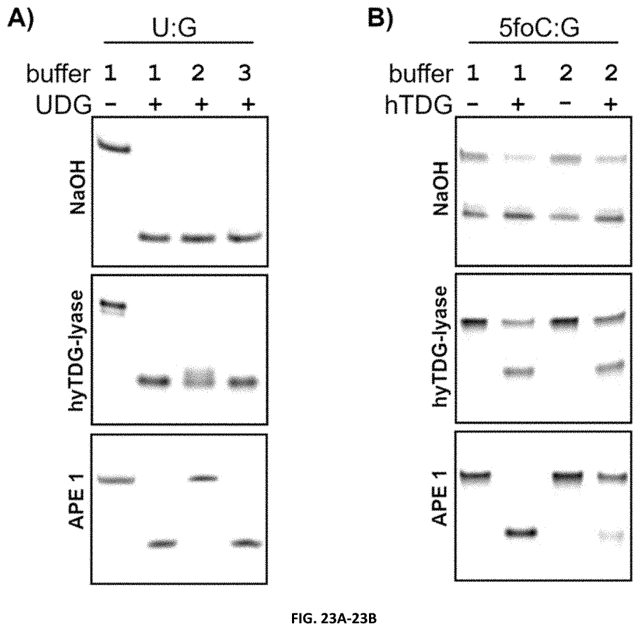

The hyTDG glycosylase is highly specific for uracil analogs mispaired with G. The Y163K mutation converts the enzyme from a glycosylase to a lyase, but would not be expected to have a substantial impact on the preference of the lyase for an abasic site opposite G. To test the opposite-base preferences of hyTDG-lyase, a single-stranded oligonucleotide containing a uracil base, as well as U:G, U:A, U:C or U:T duplexes were incubated with UDG (FIG. 24A), followed by incubation with added NaOH or hyTDG-lyase. The NaOH control showed that UDG had completely removed uracil from all the oligonucleotides. The hyTDG-lyase was able to cleave all the abasic sites from the U:G substrate, but only approximately half of the other substrates in 1 h.

To determine if the approximately 50% cleavage of the remaining substrates at 1 h was the result of a slower rate of cleavage, a real-time fluorescence assay was used in which the target oligonucleotide has a 5′-FAM label, and the complementary strand has a BHQ1-fluorescence quencher on the 3′-end. The substrate duplex contained either a U:G or a U:A base pair. Uracil was removed by UDG to generate the corresponding AP:G or AP:A abasic sites. Cleavage of the abasic site allows separation of the 5′-FAM sequence from the 3′-quencher resulting in increased fluorescence that can be measured in a qPCR machine as a function of time (FIG. 24B). Data was obtained for three different experiments. Data was fit to a single exponential where Y=A(1−e^{-kt}), Y is normalized fluorescence, A is the normalized maximum percent fluorescence, k is the apparent rate constant (min⁻¹) and t is time (min). The average k for AP:G was observed to be 0.0569+/−0.011 min⁻¹, and for AP:A 0.123+/−0.002 min⁻¹. In both cases, cleavage went to completion by 200 min, and the rate of the AP:G cleavage is roughly twice that of AP:A (FIG. 24B), in accord with the gel electrophoresis results (FIG. 24A).

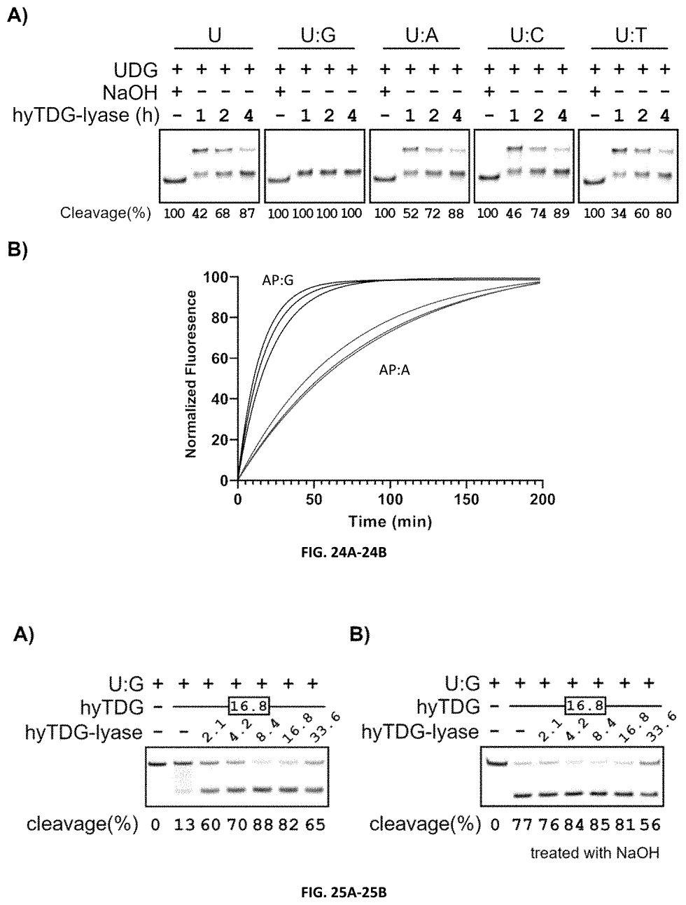

To determine if the hyTDG glycosylase and the hyTDG-lyase could be used together to cleave substrates containing U:G mispairs, a series of experiments was performed where the molar ratio of the two proteins was varied. A 5′-FAM labelled, U:G-containing duplex (2.5 pmol) was incubated in TDG buffer at 65° C. with 16.8 pmol hyTDG and increasing amounts of hyTDG-lyase for 1 h. The progress of the reaction was monitored by gel electrophoresis (FIG. 25A). Increasing hyTDG-lyase resulted in more overall cleavage with a maximum of 88% with 8.4 pmol of hyTDG lyase. With two equivalents of hyTDG-lyase, overall cleavage dropped to 65%, suggesting that the hyTDG-lyase could bind to the U:G site, blocking glycosylase cleavage by hyTDG. To further examine potential competition between hyTDG and hyTDG-lyase, the above experiment was repeated but after 1 h, NaOH was added to cleave all abasic sites (FIG. 25B). Comparing A and B, increases cleavage is observed following addition of NaOH when 2.1 pmoles of hyTDG-lyase was in the reaction. This result suggests that hyTDG could excise the U but remain bound to the abasic site product, blocking subsequent cleavage by the hyTDG-lyase. When the mole ratio of the lyase was twice that of the glycosylase, the hyTDG-lyase could bind to the U:G mispair, preventing hyTDG excision of U. Empirically, the most cleavage was observed when the ratio of the glycosylase to lyase was approximately two.

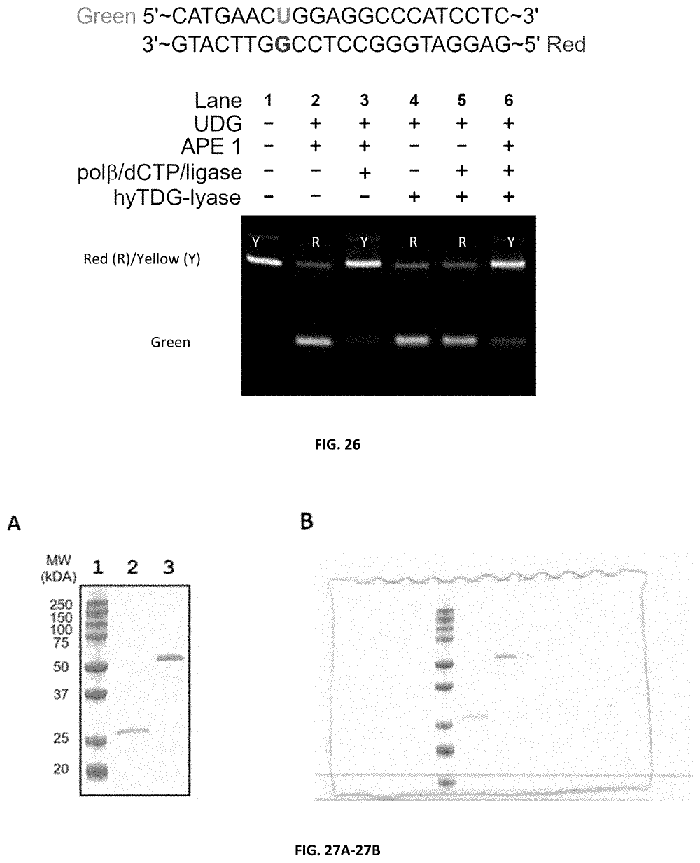

In a final experiment, the inventors examined the participation of hyTDG-lyase in a short patch base excision repair (SP-BER) cycle (FIG. 26). A dual fluorescent duplex system was constructed in which the U-containing strand was labelled with fluorescein on the 5′-end (green) and the complementary strand with Cy5 on its 5′-end (red). When both oligonucleotides comigrate on the gel, the band appears yellow in color (FIG. 26, lane 1). As a positive control, incubation with UDG and APE 1 results in cleavage of the U-containing strand and the appearance of a lower, green gel band (FIG. 26, lane 2). Addition of polymerase β (pol β) and dCTP results in the insertion of C opposite G and DNA ligase completes repair of the phosphodiester backbone (FIG. 26, lane 3). Addition of UDG and hyTDG-lyase results in the removal of uracil and cleavage of the abasic site (FIG. 26, lane 4). In contrast to the previous example, however, the addition of the repair complex comprised of DNA polβ, dCTP and DNA ligase does not result in completed repair. As shown in FIG. 19, the hyTDG-lyase cleaves on the 3′-side of the basic site, generating a substrate that can be neither ligated or extended (FIG. 26, lane 5). Addition of APE 1 allows cleavage of the fragment remaining bound to the 3′-end, generating an extendable 3′-hydroxyl.

DNA repair enzymes are essential for protecting the human genome (1-5). DNA repair enzymes are also potential pharmacological targets in the treatment of infectious diseases and cancer (6-10). The repair of endogenous DNA damage is usually accomplished by the BER pathway. The BER pathway is initiated by a series of lesion-specific glycosylases that recognize and excise single-base lesion from the DNA generating an abasic site. The resulting abasic sites can then be cleaved by lyases or endonucleases. If a 3′-hydroxyl is present at the repair gap, a dNTP can be inserted by a polymerase, and if a 5′-phosphate is present the nick can be ligated by a DNA ligase completing the repair cycle (FIG. 19).

Most glycosylase assays require not only base excision, but cleavage of the abasic site as well. Cleaved DNA fragments can easily be separated by gel electrophoresis or chromatography and quantified. Multiple approaches for oligonucleotide cleavage have been used in such assays in the past including the addition of endonucleases, bifunctional glycosylase lyases, and alkaline-induced β-elimination. A significant challenge, however, is that various enzymes are active in different buffers, and finding the right combination of glycosylase, lyase buffer, and temperature can be challenging. The addition of NaOH following a glycosylase reaction is an effective method for cleaving the backbone, however, some modified bases of biological interest are themselves alkaline labile (16-19), resulting in false positive results. Additionally, added NaOH, particularly in the presence of tris buffer, can interfere with gel electrophoresis.

Previously, Begley and Cunningham showed that a single Y to K mutation could abolish the glycosylase activity of MIG and convert it to a lyase (25). We therefore made the corresponding Y163K mutation to our hyTDG to generate the hyTDG-lyase. We confirmed the amino acid sequence of the recombinant protein using nLC-MS/MS analysis of the tryptic peptides generated by trypsin digestion (FIG. 20B).

To examine the mode of cleavage of abasic site-containing oligonucleotides, cleavage fragments were examined using MALDI-Tof-Tof-MS (FIG. 21). Nucleases and lyases can cleave on the 5′-side (i.e. APE 1) or the 3′-side (i.e., endo III), generating a variety of 3′-ends. Upon the basis of the observed MALDI-Tof-Tof-MS fragments, it was determined that hyTDG-lyase cleaves on the 3′-side of the abasic site (FIG. 21). The mass of the observed product was, however, 60 mass unit higher than expected. This mass difference was attributed to an adduct with β-mercaptoethanol present in the purification buffer as shown in FIG. 21.

Using a 5′-FAM labeled oligonucleotide duplex containing an abasic site generated by UDG cleavage of a U:G mispair, the hyTDG-lyase was found to be active from 25° C. to 95° C. In contrast, the endonuclease APE 1 was not active above 45° C., and the bifunctional glycosylase/lyase Fpg was active only to 55° C. The thermal stability of the hyTDG-lyase and extended range of activity across a span of temperature could make this enzyme valuable in thermal cycling and other applications.

The inventors found that the hyTDG is active in multiple buffers including the buffer used for TDG (10 mM K₂PO₄, 30 mM NaCl, 40 mM KCl, pH 7.8) as well as the common buffer for UDG (20 mM tris-HCl, 1 mM DTT, 1 mM EDTA). In contrast APE 1 is active in TDG buffer and NEBuffer™ 1, but not UDG buffer.

The inventors examined the cleavage of oligonucleotides containing 5foC under a variety of conditions. Derivatives of 5mC, generated by Tet mediated oxidation, including 5foC, are putative intermediates in epigenetic reprogramming pathways in mammals (29-32). 5foC is demonstrated as alkaline labile, in accord with a previous report (19) and therefore if alkaline cleavage is used, cleaved bands will be observed in the absence of enzymes. In both TDG and UDG buffers, hTDG can excise 5foC and the resulting abasic site can be cleaved by hyTDG-lyase. The combination of hyTDG or APE 1 with TDG buffer generates overall greater cleavage, however, in UDG buffer, APE 1 cleavage is significantly diminished. Previously, it was shown that APE 1 could enhance the activity of hTDG by displacing it from an abasic site and facilitating turnover (33), in accord with the results reported here. The data does not suggest, however, that hyTDG-lyase can facilitate hTDG turnover.

The hyTDG glycosylase is highly specific to uracil analogs mispaired with G. It was suspected that the hyTDG-lyase would also retain affinity for mispairs with G. Cleavage of abasic sites opposite G, A, C and T as well as an abasic site in single-stranded DNA were examined. Under conditions where hyTDG-lyase completely cleaves an abasic site opposite G at 1 h, the other substrates are cleaved at or less than 50%. hyTDG-lyase cleavage of AP:A and AP:G was examined using a real-time fluorescence assay. The rate of AP:A cleavage is approximately 50% of that for AP:G cleavage, consistent with the gel assays. Assay conditions with therefore require careful consideration for using hyTDG-lyase as a general lyase. However, if the target is deaminated cytosine analogs mispaired with G, shorter reaction times would function well.

The inventor also investigated whether the combination of hyTDG and hyTDG-lyase could facilitate the cleavage of DNA containing mispairs of interest to cancer etiology or if they might inhibit one another due to their affinity for U:G and T:G mispairs. The inventors found that hyTDG and hyTDG-lyase can function together, with optimum cleavage at a mole ratio of 2 to 1. When compared to cleavage induced by alkali, the data suggest that at a ratio of hyTDG to hyTDG-lyase of 8 to 1, hyTDG can occupy an abasic site, blocking hyTDG-lyase cleavage. When using both enzymes, cleavage is optimal at a 2 to 1 ratio. If the hyTDG-lyase is present at greater than a 2 to 1 ratio over the glycosylase, the hyTDG-lyase can occupy a U:G or T:G site, blocking the activity of the hyTDG glycosylase.

In a final study the inventors examined a complete BER cycle using a dual fluorescent reporter system. In this system using a U:G substrate, incubation with UDG, APE 1, polβ, dCTP and DNA ligase results in uracil excision, cleavage of the abasic site, repair synthesis and ligation. When incubated with UDG and hyTDG, a repair gap is formed, but repair synthesis cannot occur due to the sugar fragment blocking the 3′-hydroxyl of the repair gap. Addition of APE 1 can remove the blocking sugar fragment, allowing completion of the BER cycle. The different properties of APE 1 and hyTDG-lyase could potentially be exploited in assays quantifying specific types of DNA damage, for example, those that rely upon the incorporation of fluorescent or biotinylated dNTP analogs (34-37). The hyTDG-lyase described here could be a valuable tool for examining glycosylase activity and potential pharmacological inhibition, identifying DNA damage at sequence resolution as well as preparing DNA for NGS sequencing studies.

B. Methods and Procedures

DNA repair enzymes Uracil-DNA Glycosylase (UDG, #M0280S), human Apurinic/apyrimidinic Endonuclease 1 (APE 1, #M0282S), Formamidopyrimidine DNA Glycosylase (Fpg, #M0240S) and E. coli DNA ligase (ligase, #M0205S), Endonuclease III (Endo III, #M0268S) were obtained from New England Biolabs (NEB). Human DNA polymerase β (polβ, #NBP1-72434-0.5 mg) was purchased from Novus Biologicals. The hTDG (27) and the hyTDG (20) were prepared as previously described.

The following buffers were used in this study: CutSmart™ buffer (NEB, #B6004): 50 mM potassium phosphate, 20 mM tris-acetate, 10 mM magnesium acetate, 100 mg/mL bovine serum albumin, pH 7.9; UDG buffer (NEB, #B0280SVIAL): 20 mM tris-hydrochloric acid, 1 mM dithiothreitol, 1 mM EDTA, pH 8.0; NEBuffer™1 (NEB, #B7001): 1 mM dithiothreitol, 10 mM bis tris-propane hydrochloric acid, 10 mM magnesium chloride, pH 7.0; TDG buffer: 10 mM dipotassium hydrogen phosphate, 30 mM sodium chloride, 40 mM potassium chloride, pH 7.7.

Preparation of the expression vector, and site directed mutagenesis to generate hyTDG-lyase. To introduce Y163K point mutation to hyTDG (20), site directed mutagenesis PCR was performed using a Q5 Site-Directed Mutagenesis Kit (NEB, #E0554) and pET-28a(+)-his-hyTDG plasmid DNA as template, and with forward primer 5′-TGTGGGCAAAAAAACCTGCGCGG-3′ (SEQ ID NO: 190), where desired bases are underlined, and reverse primer 5′-CCCGGCAGATCCAGAATCG-3′ (SEQ ID NO:191) according to the manufacturer's protocol for the kit, with an annealing temperature of 69° C. A fraction of the PCR product was used for kinase/ligation/digestion reactions and further transformed into DH5a competent cells provided with the kit according to the manufacturer's protocol. Antibiotic resistant clones were selected on Luria broth (LB)-agar plates containing kanamycin (50 μg/mL) and inoculated in 5 mL LB. After overnight culture, plasmid DNA was purified from the NEB® 5-alpha Competent cells, using a plasmid DNA mini prep kit (NEB, #T1010) following manufacturer's instructions. The coding sequence was confirmed by Sanger sequencing for N-terminal 6×His tagged hyTDG-lyase.

Expression and purification of hyTDG-lyase. Plasmid DNA was transformed to E. coli strain BL21 (DE3) (NEB, #C2527). Transformants were selected on agar plates (+1.4%, Fisher Scientific, #BP9723-500) containing kanamycin (50 μg/mL). Expression of the target protein was confirmed by SDS-PAGE and Coomassie brilliant blue staining in a small-scale culture after induction with IPTG (1 mM). Selected clones were further cultured in 100 mL LB (Fisher Scientific, #BP9723-500) containing kanamycin (50 μg/mL) at 37° C. on a shaker (250 rpm) until the optical density reaches to 0.4-0.8 at 600 nanometers.

Expression of his tagged hyTDG-lyase was induced with IPTG (1 mM) at 250 rpm, 30° C. for 6 hours. The cells were harvested by centrifugation at 4100 rpm for 5 min and stored −80° C. until use. The purification of the target protein was performed as previously described (20) with slight modification. Briefly, the cell pellet was thawed and suspended in 4 mL of lysis buffer and sonicated on ice. After removal of cell debris by centrifugation, supernatant was loaded on previously equilibrated HisPur Ni-NTA Resin (Thermo Scientific, #88221) and incubated for 1.5 h at 4° C. on a see-saw shaker. The suspension of HisPur Ni NTA Resin beads and cell lysate was centrifuged using centrifuge column (Pierce, #89896) at 1000 g, 4° C. for 5 min. The beads were washed with 3 mL of wash buffer A (2×), 3 mL of wash buffer B (2×), and 3 mL of wash buffer C (6×). The bound protein was eluted from the beads in 1.2 mL of elution buffer. The protein concentration was quantified with a Bradford protein assay (Bio-Rad, #5000006) using bovine serum albumin as a standard. The purified protein was resolved by gel electrophoresis (12% Tris-Glycine PAGE (Bio-Rad, #4561044) and Coomassie blue staining) and the purity of the target protein band was determined by densitometry using ImageJ software (version 1.53e), using picture obtained after separation of the protein.

Proteomic verification of protein sequence. Proteomics performed as previously described (20). Ten micrograms of hyTDG-lyase protein were separated in SDS-PAGE. The gel bands with molecular weight around 26.5 kDa were removed from the gel and destained with 50% methanol in water. Gel bands were dried under reduced pressure and suspended in 50 μL of acetic anhydride and 200 μL of acetic acid to acetylate protein lysine residues and incubated at 37° C. on a shaker for 1 h. Liquid was decanted and the gel bands were washed three times with deionized water (1 mL). Washed gel bands were dried and ground into a fine powder with a tip-sealed 200 μL pipette tip. One-hundred microliter buffer (50 mM NH₄HCO₃) was added, and the pH of the resultant jelly was adjusted to be approximate 8 using NH₃.H₂O. Two microgram of trypsin was added to the sample and digested over-night at 37° C. Digested peptides were extracted with acetonitrile, dried, and resuspended in 50 μL of 1% formic acid for nLC-MS/MS analysis.

Peptide mixtures were separated by reversed-phase liquid chromatography using an Easy-nanoLC equipped with an autosampler (Thermo Fisher Scientific). A PicoFrit 25 cm length×75-μm id, ProteoPep™ analytical column packed with a mixed (1:1) packing material (Waters XSelect HSS T3, 5μ, and Waters YMC ODS-AQ, S-5, 100 Å) was used to separate peptides by reversed-phase liquid chromatography (solvent A, 0.1% formic acid in water; solvent B, 0.1% formic acid in acetonitrile), with a 100 min gradient from 2 to 45% of solvent B with a flow rate of 300 μL/min. The QExactive mass analyzer was set to acquire data at a resolution of 35,000 in full scan mode and 17,500 in MS/MS mode. The top 15 most intense ions in each MS survey scan were automatically selected for MS/MS.

Peptides were identified with PEAK® 8.5 (Bioinformatics Solutions Inc., On, Canada) to perform a de novo sequencing assisted database search against the hyTDG-lyase protein sequence. Acetylation of lysine, serine, threonine, cysteine, tyrosine and histidine (K, S, T, C, Y and H), oxidation of methionine and deamination of asparagine and glutamine were set as variable modifications. The false discovery rate (FDR) was estimated by the ratio of decoy hits over target hits among peptide spectrum match (PSMs). The maximum allowed −10 log P is >=15.

Oligonucleotide synthesis. All oligonucleotides were synthesized on an Expedite 8909 synthesizer using phosphoramidites from Glen Research (Sterling, Va.). 5′-FAM labelled 18 base oligonucleotides containing U or T were synthesized using standard phosphoramidites (Bz-dA, Bz-dC, iBu-dG, dT) and a 6-fluorescein (FAM) phosphoramidite without DMT. 3′BHQ1 CPG column was used for the synthesis of complementary G oligonucleotide. The oligonucleotides were deprotected in ammonium hydroxide at 60° C. for 15 h. A 5′-FAM labelled 18 base oligonucleotides containing 5foC was synthesized using standard phosphoramidites (Bz-dA, Bz-dC, dT), dmf-dG and a 6-fluorescein phosphoramidite with DMT. Oligonucleotide were deprotected in ammonium hydroxide at room temperature for 17 h.

HPLC purification of oligonucleotides was performed on a Hewlett Packard 1050 HPLC with a PDA detector. DMT-on oligonucleotides were purified using a Hamilton PRP-1 column (10×250 mm) and a gradient of acetonitrile in 10 mM potassium phosphate, pH 7.4. Detritylation of complementary G and 5foC oligonucleotides were performed using 2% trifluoroacetic acid and 0.4% acetic acid, respectively. DMT-off oligonucleotides were purified using a Phenomenex Clarity-RP column (4.6×250 mm) and a gradient of acetonitrile in water.

Glycosylase assays. Annealed oligonucleotides (U:G, T:G or 5foC:G, 2.5 pmol) were incubated with enzymes, UDG (2.5 units, 37° C.), hyTDG (16.8 pmol, 65° C.) or hTDG (31 pmol, 37° C.) for 1 h. Reactions for UDG were performed in 1×UDG buffer, and hTDG and hyTDG reactions in 1×TDG buffer, otherwise as mentioned specifically.

To perform sequential reactions with a glycosylase and a lyase, oligonucleotides (2.5 pmol) were incubated with a glycosylase for 1 h at an appropriate temperature. Lyase reactions were performed by adding APE 1 (5 units, 37° C., 1 h) or hyTDG-lyase (0.06-33.6 pmol) at a specified temperature for 1 h. Alkaline cleavage was induced with NaOH (160 mM) 96° C., 10 min.

Gel electrophoresis. To separate 5′-FAM labelled 18 base oligonucleotides after glycosylase excision and AP-site cleavage reactions, samples were mixed with an equal volume of formamide and loaded to the 20% polyacrylamide gel containing 6 M urea and run at 180 V for 35-45 min in 1×TBE buffer. To separate the dual labeled (FAM and Cy5) 79 base oligonucleotide after repair reactions, samples were mixed with an equal volume of formamide, heated to 95° C. for 1 min and loaded onto a 15% polyacrylamide gel containing 8 M urea and run at 180 V for 50 min in 1×TBE buffer. Gels were visualized using a Storm 860 gel imager. When appropriate the FAM and Cy5 scans were adjusted for brightness and contrast, pseudo colored, and overlayed.

Real-time cleavage assay. Reactions were conducted in a total of 25 μL containing TDG buffer. Duplex oligonucleotides (25 pmol) with a U:G mispair, a 5′-FAM label and a 3′-BHQ1 quencher were pre-treated with UDG (1 unit) for 1 h at 37° C. to generate an abasic site. Samples were briefly cooled on ice and hyTDG-lyase (25 pmol) was added and each reaction was placed into a 96-well plate in a Roche 480 qPCR instrument and heated to 65° C. Fluorescence was monitored initially every 5 s for −2 min then every 40 s for the remainder of the 2 h experiment. The maximum observed fluorescence in each well was normalized to 100% at the end of the experiment.

MALDI mass spectrometry. A 20 μM stock solution containing one equivalent of an 18 base U-containing oligonucleotide and two equivalents of the complementary oligo with a G directly opposite U in TDG buffer. From this stock solution, a 5 μL aliquot (100 pmol) was treated in a 25 μL reaction containing 25 pmol of hyTDG and 12.5 pmol of hyTDG-lyase in 1×TDG buffer and heated at 65° C. for 2 h. Reaction samples were the desalted using Bio-Rad micro Bio-spin 6 columns (Hercules, Calif.), eluted, dried in vacuo, and resuspended in 5 μL distilled water with 2 μL of ammonium cation exchange resin for 40 min (37). Aliquots (1 μL) were then placed on a MALDI plate and spotted with 1 μL of 3-hydroxpicolinic acid matrix (70 mg/mL 3-HPA, 10 mg/mL diammonium citrate, in 50/50 ACN/distilled water and 0.1% trifluoracetic acid).

Samples were analyzed with a high-resolution MALDI-Tof-Tof (Bruker, MA) Ultraflextreme to identify cleavage products following glycosylase and lyase reactions. The reflectron positive ion mode was used with the ‘ultra’ laser beam parameter set, and laser fluency manually optimized for oligonucleotide standards. Pulsed Ion Extraction was set to 170 ns, IS2 voltage: 17.85 kV and Lens: 7.50 kV. Mass accuracy was calibrated using Bruker's low molecular weight oligonucleotide standard mixture prior to data acquisition using a cubic enhanced fit. A minimum of 1000 spectra were acquired per spot. The data was exported into Mmass, using the Bruker CompassXport software, and then baseline corrected and Savitsky-Golay smoothed. MALDI spectra are plotted using the PRISM software.

Short patch repair with a fluorescent oligonucleotide. Construction of 5′-FAM labelled 79 base oligonucleotide duplex was described previously (38). The upper strand was 5′-FAM labelled and contained U, while complementary strand was 5′-Cy5 labelled and contained a G opposite the U to produce a U:G mispair. An enzymatic repair reaction was performed in three sequential steps: glycosylase treatment, cleavage, and repair. Each 12.5 μl reaction initially consisted of 79 base U:G-containing oligonucleotide (2.5 pmol), UDG (2.5 units), dCTP (20 μM), NAD+ (26 μM), and 1× CutSmart™ buffer. In the glycosylase (UDG) reaction step, samples were incubated for 1 h at 37° C. to allow for removal of U and creation of AP sites. Next, cleavage was performed by adding APE 1 (5 units) or hyTDG-lyase (26.9 pmol) to the glycosylase reactions. Samples were incubated for 30 min at 37° C. to allow for cleavage of the phosphodiester backbone. Repair reactions were completed by adding polβ (6.2 pmol) and E. coli ligase (5 units) to the reaction. When indicated, APE 1 (5 units) was added to determine if APE 1 could repair the 3′ end cleaved by hyTDG-lyase and allow for extension by polβ. Samples were again incubated at 37° C. for 1 h. Finally, samples were resolved by gel electrophoresis as mentioned above.

Abbreviations. UDG, uracil-DNA glycosylase; TDG, thymine DNA glycosylase; hTDG, human TDG; hyTDG, hybrid TDG; 5foC, 5-formyl cytosine; MIG, thymine DNA glycosylase from Methanobacterium thermoautotrophicum; BER, base excision repair; 5foC, 5-formylcytosine; BHQ1, black hole fluorescence quencher 1; FAM, 6-carboxyfluorescein; MS, mass spectrometry;

### EXAMPLE 2 REFERENCES

1. Friedberg (2016) DNA Repair (Amst) 37, 35-39.
2. Howard and Wilson, (2018) DNA Repair (Amst) 71, 101-107.
3. Mullins et al., (2019) Trends Biochem Sci 44, 765-781.
4. Zhao et al., (2021) Int Rev Cell Mol Biol 364, 163-193.
5. Bordin et al., (2021) DNA Repair (Amst) 99, 103051.
6. Li et al., (2018) Oncotarget 9, 31719-31743.
7. Visnes et al., (2018) DNA Repair (Amst) 71, 118-126.
8. Kurthkoti et al., (2020) Future Med Chem 12, 339-355.
9. Hans et al., (2020) Int J Mol Sci 21, 9226.
10. Grundy and Parsons (2020) Essays Biochem 64, 831-843.
11. Briggs and Heyn, (2012) Methods in Molecular Biology 840, 143-154.
12. Do et al., (2013) Clin. Chem. 59, 1376-1383.
13. Costello et al., (2013) Nucleic Acids Res. 41, 1-12.
14. Arbeithuber et al., (2016) DNA Res. 23, 547-559.
15. Chen et al., (2017) Science. 355, 752-756.
16. D'Incalci et al., (1985) Cancer Res 45, 3197-3202.
17. Mattes et al., (1986) Biochim Biophys Acta 868, 71-76.
18. Higurashi et al., (2003) J Biol Chem 278, 51968-51973.
19. Tian et al., (2013) Chem Commun (Camb) 49, 9968-9970.
20. Hsu et al., (2022) J Biol Chem 24, 101638.
21. Coey et al., (2016) Nucleic Acids Res 44, 10248-10258.
22. Horst et al., (1996) EMBO J 15, 5459-5469.
23. Begley et al., (2003) DNA Repair (Amst) 2, 107-120.
24. Mol et al., (2002) J Mol Biol 315, 373-384.
25. Begley and Cunningham (1999) Protein Eng 12, 333-340.
26. Haldar et al., (2022) Chem Res Toxicol., 35(2):218-232.
27. Hardeland et al., (2000) J Biol Chem 275, 33449-33456.
28. Hsu et al., (2017) Trends Cancer Res 12, 111-132.
29. Pfaffeneder et al., (2011) Angew Chem Int Ed Engl 50, 7008-7012.
30. Ito et al., (2011) Science 333, 1300-1303.
31. Maiti and Drohat, (2011) J Biol Chem 286, 35334-35338.
32. Pfeifer et al., (2020) J Mol Biol 432, 1718-1730.
33. Fitzgerald and Drohat, (2008) J Biol Chem 283, 32680-32690.
34. Anderson et al., (2005) Biotechniques 38, 257-264.
35. Howell et al., (2010) Nucleic Acids Res 38, doi:10.1093.
36. Holton et al., (2018) DNA Repair (Amst) 2018; 66-67: 42-49.
37. Gassman and Holton, (2019) Curr Opin Biotechnol 55, 30-35.
37. Darwanto et al., (2009) Anal Biochem 394, 13-23.
38. Hsu et al., (2022) A Combinatorial System to Examine the Enzymatic Repair of Multiply Damaged DNA Substrates. (submitted).

1. A hybrid glycosylase polypeptide comprising an amino terminal human thymine DNA glycosylase (TDG) activator segment linked to a catalytic domain of a thermophile TDG.

2. The polypeptide of claim 1, wherein the amino terminal human activator segment has an amino acid sequence of SEQ ID NO:2 or a variant thereof.

3. The polypeptide of claim 1, wherein the catalytic domain of a thermophile TDG has an amino acid sequence that is 90% identical to SEQ ID NO:3.

4. The polypeptide of claim 1 further comprising a tag.

5. (canceled)

6. The polypeptide of claim 1, wherein the polypeptide has an amino acid sequence of SEQ ID NO:1.

7. The polypeptide of claim 1, wherein the polypeptide comprising an amino acid sequence that is 90% identical to SEQ ID NO:1.

8. (canceled)

9. The polypeptide of claim 7, wherein the polypeptide has an amino acid sequence of SEQ ID NO:1.

10. A nucleic acid encoding a polypeptide of claim 1.

11. A kit comprising a polypeptide of claim 1.

12.-16. (canceled)

17. A hybrid glycosylase polypeptide comprising an amino terminal human thymine DNA glycosylase (TDG) activator segment linked to a catalytic domain of a thermophile TDG comprising a substitution of Y126K corresponding to SEQ ID NO:3.

18. The polypeptide of claim 17, wherein the amino terminal human activator segment has an amino acid sequence of SEQ ID NO:2 or a variant thereof.

19. The polypeptide of claim 17, wherein the catalytic domain of a thermophile TDG has an amino acid sequence that is 90% identical to SEQ ID NO:189, wherein amino 155 is a lysine.

20. The polypeptide of claim 17 further comprising a tag.

21. (canceled)

22. The polypeptide of claim 17, wherein the polypeptide has an amino acid sequence of SEQ ID NO:189.

23. A hybrid lyase polypeptide comprising an amino acid sequence that is 90% identical to SEQ ID NO:189 wherein amino acid 155 is a lysine.

24. (canceled)

25. (canceled)

26. A nucleic acid encoding a polypeptide of claim 17.

27. (canceled)

28. A nucleic acid encoding a polypeptide of claim 7.

29. A nucleic acid encoding a polypeptide of claim 24.


SEQUENCE LISTING (255 sequences)

SEQ ID NO: 1 - protein, 250 aa, Artificial Sequence
  recombinant peptide
SKKSGKSAKSKEKQEKITDTFKVKRKVDRLDDATNKKRKVFVSTILTFWNTDRRDFPWRHTRDPYVILITEILLRRTTAGHVKKIYDKFFVKYKCFEDILKTPKSEIAKDIKEIGLSNQRAEQLKELARVVINDYGGRVPRNRKAILDLPGVGKYTCAAVMCLAFGKKAAMVDANFVRVINRYFGGSYENLNYNHKALWELAETLVPGGKCRDFNLGLMDFSAIICAPRKPKCEKCGMSKLCSYYEKCST

SEQ ID NO: 2 - protein, 29 aa, Artificial Sequence
  recombinant peptide
SKKSGKSAKSKEKQEKITDTFKVKRKVDR

SEQ ID NO: 3 - protein, 221 aa, Artificial Sequence
  recombinant peptide
LDDATNKKRKVFVSTILTFWNTDRRDFPWRHTRDPYVILITEILLRRTTAGHVKKIYDKFFVKYKCFEDILKTPKSEIAKDIKEIGLSNQRAEQLKELARVVINDYGGRVPRNRKAILDLPGVGKYTCAAVMCLAFGKKAAMVDANFVRVINRYFGGSYENLNYNHKALWELAETLVPGGKCRDFNLGLMDFSAIICAPRKPKCEKCGMSKLCSYYEKCST

SEQ ID NO: 4 - DNA, 780 bp, Artificial Sequence
  recombinant peptide
atggggcaccaccaccaccaccacagcaagaaaagcggcaagagcgcgaagagcaaagagaagcaggaaaaaatcaccgacaccttcaaagtgaagcgtaaagttgatcgtctggacgatgcgaccaacaagaaacgtaaagtgttcgttagcaccattctgaccttttggaacaccgaccgtcgtgattttccgtggcgtcacacccgtgacccgtatgtgatcctgattaccgagatcctgctgcgtcgtaccaccgcgggtcacgtgaagaaaatttacgacaagttctttgttaagtacaaatgcttcgaagatatcctgaagaccccgaaaagcgagattgcgaaggatatcaaagaaattggcctgagcaaccagcgtgcggagcaactgaaagaactggcgcgtgtggttatcaacgactatggtggccgtgtgccgcgtaaccgtaaggcgattctggatctgccgggtgtgggcaaatacacctgcgcggcggttatgtgcctggcgtttggcaagaaagcggcgatggttgacgcgaacttcgtgcgtgttatcaaccgttactttggtggcagctatgagaacctgaactacaaccacaaggcgctgtgggagctggcggaaaccctggttccgggtggcaaatgccgtgacttcaacctgggtctgatggattttagcgcgatcatttgcgcgccgcgtaagccgaaatgcgagaagtgcggcatgagcaaactgtgcagctactatgaaaaatgcagcacctaataa

SEQ ID NO: 5 - protein, 17 aa, Artificial Sequence
  recombinant peptide
HTRDPYVILITEILLRR

SEQ ID NO: 6 - protein, 16 aa, Artificial Sequence
  recombinant peptide
HTRDPYVILITEILLR

SEQ ID NO: 7 - protein, 28 aa, Artificial Sequence
  recombinant peptide
YFGGSYENLNYNHKALWELAETLVPGGK

SEQ ID NO: 8 - protein, 13 aa, Artificial Sequence
  recombinant peptide
DPYVILITEILLR

SEQ ID NO: 9 - protein, 15 aa, Artificial Sequence
  recombinant peptide
KVFVSTILTFWNTDR

SEQ ID NO: 10 - protein, 9 aa, Artificial Sequence
  recombinant peptide
VVINDYGGR

SEQ ID NO: 11 - protein, 14 aa, Artificial Sequence
  recombinant peptide
YFGGSYENLNYNHK

SEQ ID NO: 12 - protein, 34 aa, Artificial Sequence
  recombinant peptide
AILDLPGVGKYTCAAVMCLAFGKKAAMVDANFVR

SEQ ID NO: 13 - protein, 14 aa, Artificial Sequence
  recombinant peptide
VFVSTILTFWNTDR

SEQ ID NO: 14 - protein, 10 aa, Artificial Sequence
  recombinant peptide
AAMVDANFVR

SEQ ID NO: 15 - protein, 14 aa, Artificial Sequence
  recombinant peptide
ALWELAETLVPGGK

SEQ ID NO: 16 - protein, 19 aa, Artificial Sequence
  recombinant peptide
TPKSEIAKDIKEIGLSNQR

SEQ ID NO: 17 - protein, 10 aa, Artificial Sequence
  recombinant peptide
AAMVDANFVR

SEQ ID NO: 18 - protein, 16 aa, Artificial Sequence
  recombinant peptide
SEIAKDIKEIGLSNQR

SEQ ID NO: 19 - protein, 14 aa, Artificial Sequence
  recombinant peptide
DPYVILITEILLRR

SEQ ID NO: 20 - protein, 15 aa, Artificial Sequence
  recombinant peptide
TRDPYVILITEILLR

SEQ ID NO: 21 - protein, 13 aa, Artificial Sequence
  recombinant peptide
SKEKQEKITDTFK

SEQ ID NO: 22 - protein, 16 aa, Artificial Sequence
  recombinant peptide
TTAGHVKKIYDKFFVK

SEQ ID NO: 23 - protein, 10 aa, Artificial Sequence
  recombinant peptide
VDRLDDATNK

SEQ ID NO: 24 - protein, 11 aa, Artificial Sequence
  recombinant peptide
KAAMVDANFVR

SEQ ID NO: 25 - protein, 12 aa, Artificial Sequence
  recombinant peptide
TTAGHVKKIYDK

SEQ ID NO: 26 - protein, 11 aa, Artificial Sequence
  recombinant peptide
KAILDLPGVGK

SEQ ID NO: 27 - protein, 13 aa, Artificial Sequence
  recombinant peptide
YFGGSYENLNYNH

SEQ ID NO: 28 - protein, 16 aa, Artificial Sequence
  recombinant peptide
KVFVSTILTFWNTDRR

SEQ ID NO: 29 - protein, 13 aa, Artificial Sequence
  recombinant peptide
EKQEKITDTFKVK

SEQ ID NO: 30 - protein, 11 aa, Artificial Sequence
  recombinant peptide
EKQEKITDTFK

SEQ ID NO: 31 - protein, 15 aa, Artificial Sequence
  recombinant peptide
VFVSTILTFWNTDRR

SEQ ID NO: 32 - protein, 13 aa, Artificial Sequence
  recombinant peptide
GKKAAMVDANFVR

SEQ ID NO: 33 - protein, 24 aa, Artificial Sequence
  recombinant peptide
YTCAAVMCLAFGKKAAMVDANFVR

SEQ ID NO: 34 - protein, 12 aa, Artificial Sequence
  recombinant peptide
VSTILTFWNTDR

SEQ ID NO: 35 - protein, 8 aa, Artificial Sequence
  recombinant peptide
VVINDYGG

SEQ ID NO: 36 - protein, 11 aa, Artificial Sequence
  recombinant peptide
DIKEIGLSNQR

SEQ ID NO: 37 - protein, 12 aa, Artificial Sequence
  recombinant peptide
QEKITDTFKVKR

SEQ ID NO: 38 - protein, 12 aa, Artificial Sequence
  recombinant peptide
WELAETLVPGGK

SEQ ID NO: 39 - protein, 11 aa, Artificial Sequence
  recombinant peptide
KVDRLDDATNK

SEQ ID NO: 40 - protein, 9 aa, Artificial Sequence
  recombinant peptide
QEKITDTFK

SEQ ID NO: 41 - protein, 12 aa, Artificial Sequence
  recombinant peptide
KVDRLDDATNKK

SEQ ID NO: 42 - protein, 10 aa, Artificial Sequence
  recombinant peptide
IYDKFFVKYK

SEQ ID NO: 43 - protein, 11 aa, Artificial Sequence
  recombinant peptide
KIYDKFFVKYK

SEQ ID NO: 44 - protein, 11 aa, Artificial Sequence
  recombinant peptide
QEKITDTFKVK

SEQ ID NO: 45 - protein, 9 aa, Artificial Sequence
  recombinant peptide
ITDTFKVKR

SEQ ID NO: 46 - protein, 9 aa, Artificial Sequence
  recombinant peptide
KIYDKFFVK

SEQ ID NO: 47 - protein, 9 aa, Artificial Sequence
  recombinant peptide
AEQLKELAR

SEQ ID NO: 48 - protein, 8 aa, Artificial Sequence
  recombinant peptide
EIGLSNQR

SEQ ID NO: 49 - protein, 10 aa, Artificial Sequence
  recombinant peptide
TILTFWNTDR

SEQ ID NO: 50 - protein, 11 aa, Artificial Sequence
  recombinant peptide
TPKSEIAKDIK

SEQ ID NO: 51 - protein, 23 aa, Artificial Sequence
  recombinant peptide
AILDLPGVGKYTCAAVMCLAFGK

SEQ ID NO: 52 - protein, 9 aa, Artificial Sequence
  recombinant peptide
ILTFWNTDR

SEQ ID NO: 53 - protein, 11 aa, Artificial Sequence
  recombinant peptide
TILTFWNTDRR

SEQ ID NO: 54 - protein, 8 aa, Artificial Sequence
  recombinant peptide
LDDATNKK

SEQ ID NO: 55 - protein, 8 aa, Artificial Sequence
  recombinant peptide
IYDKFFVK

SEQ ID NO: 56 - protein, 14 aa, Artificial Sequence
  recombinant peptide
IAKDIKEIGLSNQR

SEQ ID NO: 57 - protein, 9 aa, Artificial Sequence
  recombinant peptide
LDDATNKKR

SEQ ID NO: 58 - protein, 7 aa, Artificial Sequence
  recombinant peptide
LDDATNK

SEQ ID NO: 59 - protein, 12 aa, Artificial Sequence
  recombinant peptide
VVINDYGGRVPR

SEQ ID NO: 60 - protein, 24 aa, Artificial Sequence
  recombinant peptide
KAILDLPGVGKYTCAAVMCLAFGK

SEQ ID NO: 61 - protein, 7 aa, Artificial Sequence
  recombinant peptide
EIGLSNQ

SEQ ID NO: 62 - protein, 6 aa, Artificial Sequence
  recombinant peptide
FFVKYK

SEQ ID NO: 63 - protein, 10 aa, Artificial Sequence
  recombinant peptide
SAKSKEKQEK

SEQ ID NO: 64 - protein, 8 aa, Artificial Sequence
  recombinant peptide
ITDTFKVK

SEQ ID NO: 65 - protein, 6 aa, Artificial Sequence
  recombinant peptide
ITDTFK

SEQ ID NO: 66 - protein, 11 aa, Artificial Sequence
  recombinant peptide
VDRLDDATNKK

SEQ ID NO: 67 - protein, 8 aa, Artificial Sequence
  recombinant peptide
SEIAKDIK

SEQ ID NO: 68 - protein, 6 aa, Artificial Sequence
  recombinant peptide
RDFPWR

SEQ ID NO: 69 - protein, 8 aa, Artificial Sequence
  recombinant peptide
MVDANFVR

SEQ ID NO: 70 - protein, 8 aa, Artificial Sequence
  recombinant peptide
TPKSEIAK

SEQ ID NO: 71 - protein, 8 aa, Artificial Sequence
  recombinant peptide
TTAGHVKK

SEQ ID NO: 72 - protein, 7 aa, Artificial Sequence
  recombinant peptide
TFWNTDR

SEQ ID NO: 73 - protein, 13 aa, Artificial Sequence
  recombinant peptide
KAILDLPGVGKYT

SEQ ID NO: 74 - protein, 9 aa, Artificial Sequence
  recombinant peptide
DFNLGLMDF

SEQ ID NO: 75 - protein, 12 aa, Artificial Sequence
  recombinant peptide
KAILDLPGVGKY

SEQ ID NO: 76 - protein, 10 aa, Artificial Sequence
  recombinant peptide
SGKSAKSKEK

SEQ ID NO: 77 - protein, 13 aa, Artificial Sequence
  recombinant peptide
VSTILTFWNTDRR

SEQ ID NO: 78 - protein, 10 aa, Artificial Sequence
  recombinant peptide
VILITEILLR

SEQ ID NO: 79 - protein, 7 aa, Artificial Sequence
  recombinant peptide
VDANFVR

SEQ ID NO: 80 - protein, 13 aa, Artificial Sequence
  recombinant peptide
TTAGHVKKIYDKF

SEQ ID NO: 81 - protein, 6 aa, Artificial Sequence
  recombinant peptide
EIGLSN

SEQ ID NO: 82 - protein, 7 aa, Artificial Sequence
  recombinant peptide
LCSYYEK

SEQ ID NO: 83 - protein, 10 aa, Artificial Sequence
  recombinant peptide
IKEIGLSNQR

SEQ ID NO: 84 - protein, 7 aa, Artificial Sequence
  recombinant peptide
TTAGHVK

SEQ ID NO: 85 - protein, 9 aa, Artificial Sequence
  recombinant peptide
AMVDANFVR

SEQ ID NO: 86 - protein, 9 aa, Artificial Sequence
  recombinant peptide
VFVSTILTF

SEQ ID NO: 87 - protein, 22 aa, Artificial Sequence
  recombinant peptide
KAILDLPGVGKYTCAAVMCLAF

SEQ ID NO: 88 - protein, 9 aa, Artificial Sequence
  recombinant peptide
KSGKSAKSK

SEQ ID NO: 89 - protein, 8 aa, Artificial Sequence
  recombinant peptide
SGKSAKSK

SEQ ID NO: 90 - protein, 5 aa, Artificial Sequence
  recombinant peptide
DFPWR

SEQ ID NO: 91 - protein, 13 aa, Artificial Sequence
  recombinant peptide
YTCAAVMCLAFGK

SEQ ID NO: 92 - protein, 7 aa, Artificial Sequence
  recombinant peptide
ITEILLR

SEQ ID NO: 93 - protein, 5 aa, Artificial Sequence
  recombinant peptide
AEQLK

SEQ ID NO: 94 - protein, 11 aa, Artificial Sequence
  recombinant peptide
VKKIYDKFFVK

SEQ ID NO: 95 - protein, 8 aa, Artificial Sequence
  recombinant peptide
TFWNTDRR

SEQ ID NO: 96 - protein, 8 aa, Artificial Sequence
  recombinant peptide
KVFVSTIL

SEQ ID NO: 97 - protein, 12 aa, Artificial Sequence
  recombinant peptide
CGMSKLCSYYEK

SEQ ID NO: 98 - protein, 10 aa, Artificial Sequence
  recombinant peptide
ILTFWNTDRR

SEQ ID NO: 99 - protein, 5 aa, Artificial Sequence
  recombinant peptide
SEIAK

SEQ ID NO: 100 - protein, 10 aa, Artificial Sequence
  recombinant peptide
KVFVSTILTF

SEQ ID NO: 101 - protein, 7 aa, Artificial Sequence
  recombinant peptide
AAMVDAN

SEQ ID NO: 102 - protein, 6 aa, Artificial Sequence
  recombinant peptide
YFGGSY

SEQ ID NO: 103 - protein, 7 aa, Artificial Sequence
  recombinant peptide
TLVPGGK

SEQ ID NO: 104 - protein, 10 aa, Artificial Sequence
  recombinant peptide
VFVSTILTFW

SEQ ID NO: 105 - protein, 10 aa, Artificial Sequence
  recombinant peptide
KVDRLDDATN

SEQ ID NO: 106 - protein, 6 aa, Artificial Sequence
  recombinant peptide
VVINDY

SEQ ID NO: 107 - protein, 7 aa, Artificial Sequence
  recombinant peptide
DLPGVGK

SEQ ID NO: 108 - protein, 7 aa, Artificial Sequence
  recombinant peptide
SAKSKEK

SEQ ID NO: 109 - protein, 7 aa, Artificial Sequence
  recombinant peptide
SKEKQEK

SEQ ID NO: 110 - protein, 5 aa, Artificial Sequence
  recombinant peptide
WNTDR

SEQ ID NO: 111 - protein, 6 aa, Artificial Sequence
  recombinant peptide
FEDILK

SEQ ID NO: 112 - protein, 5 aa, Artificial Sequence
  recombinant peptide
TDTFK

SEQ ID NO: 113 - protein, 7 aa, Artificial Sequence
  recombinant peptide
KSGKSAK

SEQ ID NO: 114 - protein, 11 aa, Artificial Sequence
  recombinant peptide
KPKCEKCGMSK

SEQ ID NO: 115 - protein, 6 aa, Artificial Sequence
  recombinant peptide
DFNLGL

SEQ ID NO: 116 - protein, 7 aa, Artificial Sequence
  recombinant peptide
IGLSNQR

SEQ ID NO: 117 - protein, 8 aa, Artificial Sequence
  recombinant peptide
LDLPGVGK

SEQ ID NO: 118 - protein, 7 aa, Artificial Sequence
  recombinant peptide
VFVSTIL

SEQ ID NO: 119 - protein, 10 aa, Artificial Sequence
  recombinant peptide
KQEKITDTFK

SEQ ID NO: 120 - protein, 6 aa, Artificial Sequence
  recombinant peptide
LDDATN

SEQ ID NO: 121 - protein, 12 aa, Artificial Sequence
  recombinant peptide
VDRLDDATNKKR

SEQ ID NO: 122 - protein, 14 aa, Artificial Sequence
  recombinant peptide
KELARVVINDYGGR

SEQ ID NO: 123 - protein, 5 aa, Artificial Sequence
  recombinant peptide
KIYDK

SEQ ID NO: 124 - protein, 8 aa, Artificial Sequence
  recombinant peptide
KAAMVDAN

SEQ ID NO: 125 - protein, 8 aa, Artificial Sequence
  recombinant peptide
VINDYGGR

SEQ ID NO: 126 - protein, 6 aa, Artificial Sequence
  recombinant peptide
SGKSAK

SEQ ID NO: 127 - protein, 6 aa, Artificial Sequence
  recombinant peptide
HTRDPY

SEQ ID NO: 128 - protein, 6 aa, Artificial Sequence
  recombinant peptide
KVFVST

SEQ ID NO: 129 - protein, 10 aa, Artificial Sequence
  recombinant peptide
EKQEKITDTF

SEQ ID NO: 130 - protein, 5 aa, Artificial Sequence
  recombinant peptide
ITDTF

SEQ ID NO: 131 - protein, 6 aa, Artificial Sequence
  recombinant peptide
FWNTDR

SEQ ID NO: 132 - protein, 7 aa, Artificial Sequence
  recombinant peptide
INDYGGR

SEQ ID NO: 133 - protein, 7 aa, Artificial Sequence
  recombinant peptide
VKKIYDK

SEQ ID NO: 134 - protein, 9 aa, Artificial Sequence
  recombinant peptide
RTTAGHVKK

SEQ ID NO: 135 - protein, 5 aa, Artificial Sequence
  recombinant peptide
FFVKY

SEQ ID NO: 136 - protein, 5 aa, Artificial Sequence
  recombinant peptide
VFVST

SEQ ID NO: 137 - protein, 5 aa, Artificial Sequence
  recombinant peptide
DATNK

SEQ ID NO: 138 - protein, 7 aa, Artificial Sequence
  recombinant peptide
TAGHVKK

SEQ ID NO: 139 - protein, 5 aa, Artificial Sequence
  recombinant peptide
FVKYK

SEQ ID NO: 140 - protein, 9 aa, Artificial Sequence
  recombinant peptide
EIGLSNQRA

SEQ ID NO: 141 - protein, 5 aa, Artificial Sequence
  recombinant peptide
TTAGH

SEQ ID NO: 142 - protein, 6 aa, Artificial Sequence
  recombinant peptide
NDYGGR

SEQ ID NO: 143 - protein, 13 aa, Artificial Sequence
  recombinant peptide
EIGLSNQRAEQLK

SEQ ID NO: 144 - protein, 5 aa, Artificial Sequence
  recombinant peptide
RKVDR

SEQ ID NO: 145 - protein, 7 aa, Artificial Sequence
  recombinant peptide
DATNKKR

SEQ ID NO: 146 - protein, 6 aa, Artificial Sequence
  recombinant peptide
WNTDRR

SEQ ID NO: 147 - protein, 6 aa, Artificial Sequence
  recombinant peptide
LVPGGK

SEQ ID NO: 148 - protein, 5 aa, Artificial Sequence
  recombinant peptide
KFFVK

SEQ ID NO: 149 - protein, 12 aa, Artificial Sequence
  recombinant peptide
AILDLPGVGKYT

SEQ ID NO: 150 - protein, 12 aa, Artificial Sequence
  recombinant peptide
IGLSNQRAEQLK

SEQ ID NO: 151 - protein, 31 aa, Artificial Sequence
  recombinant peptide
YFGGSYENLNYNHKALWELAETLVPGGKCRD

SEQ ID NO: 152 - protein, 7 aa, Artificial Sequence
  recombinant peptide
AEQLKEL

SEQ ID NO: 153 - protein, 9 aa, Artificial Sequence
  recombinant peptide
FSAIICAPR

SEQ ID NO: 154 - protein, 16 aa, Artificial Sequence
  recombinant peptide
DIKEIGLSNQRAEQLK

SEQ ID NO: 155 - protein, 5 aa, Artificial Sequence
  recombinant peptide
VPGGK

SEQ ID NO: 156 - protein, 6 aa, Artificial Sequence
  recombinant peptide
RTTAGH

SEQ ID NO: 157 - protein, 5 aa, Artificial Sequence
  recombinant peptide
KELAR

SEQ ID NO: 158 - protein, 17 aa, Artificial Sequence
  recombinant peptide
INRYFGGSYENLNYNHK

SEQ ID NO: 159 - protein, 5 aa, Artificial Sequence
  recombinant peptide
KVFVS

SEQ ID NO: 160 - protein, 5 aa, Artificial Sequence
  recombinant peptide
DYGGR

SEQ ID NO: 161 - protein, 6 aa, Artificial Sequence
  recombinant peptide
KIYDKF

SEQ ID NO: 162 - protein, 10 aa, Artificial Sequence
  recombinant peptide
DFSAIICAPR

SEQ ID NO: 163 - protein, 6 aa, Artificial Sequence
  recombinant peptide
GLSNQR

SEQ ID NO: 164 - protein, 13 aa, Artificial Sequence
  recombinant peptide
NRKAILDLPGVGK

SEQ ID NO: 165 - protein, 10 aa, Artificial Sequence
  recombinant peptide
INDYGGRVPR

SEQ ID NO: 166 - protein, 9 aa, Artificial Sequence
  recombinant peptide
HHHHHHSKK

SEQ ID NO: 167 - protein, 7 aa, Artificial Sequence
  recombinant peptide
DDATNKK

SEQ ID NO: 168 - protein, 5 aa, Artificial Sequence
  recombinant peptide
EKQEK

SEQ ID NO: 169 - protein, 8 aa, Artificial Sequence
  recombinant peptide
EQLKELAR

SEQ ID NO: 170 - protein, 5 aa, Artificial Sequence
  recombinant peptide
PGVGK

SEQ ID NO: 171 - protein, 258 aa, Artificial Sequence
  recombinant peptide
MGHHHHHHSKKSGKSAKSKEKQEKITDTFKVKRKVDRLDDATNKKRKVFVSTILTFWNTDRRDFPWRHTRDPYVILITEILLRRTTAGHVKKIYDKFFVKYKCFEDILKTPKSEIAKDIKEIGLSNQRAEQLKELARVVINDYGGRVPRNRKAILDLPGVGKYTCAAVMCLAFGKKAAMVDANFVRVINRYFGGSYENLNYNHKALWELAETLVPGGKCRDFNLGLMDFSAIICAPRKPKCEKCGMSKLCSYYEKCST

SEQ ID NO: 172 - protein, 7 aa, Artificial Sequence
  recombinant peptide
LPGVGKY

SEQ ID NO: 173 - protein, 6 aa, Artificial Sequence
  recombinant peptide
  misc_feature: X is lysine K or serine S
  misc_feature (6)..(6): Xaa can be any naturally occurring amino acid
LPGVGX

SEQ ID NO: 174 - protein, 17 aa, Artificial Sequence
  recombinant peptide
DFNLGLMDFSAIICAPR

SEQ ID NO: 175 - protein, 8 aa, Artificial Sequence
  recombinant peptide
DYKDDDDK

SEQ ID NO: 176 - protein, 8 aa, Artificial Sequence
  recombinant peptide
DLYDDDDK

SEQ ID NO: 177 - protein, 8 aa, Artificial Sequence
  recombinant peptide
MGHHHHHH

SEQ ID NO: 178 - DNA, 18 bp, Artificial Sequence
  synthetic oligonucleotide
  misc_feature: n is any nucleotide
  misc_feature (7)..(7): n is a, c, g, or t
cgtggcnggccacgacgg

SEQ ID NO: 179 - DNA, 18 bp, Artificial Sequence
  synthetic oligonucleotide
ccgtcgtggccggccacg

SEQ ID NO: 180 - DNA, 18 bp, Artificial Sequence
  synthetic oligonucleotide
  misc_feature (7)..(7): n = U
cgtggcnggccacgacgg

SEQ ID NO: 181 - DNA, 18 bp, Artificial Sequence
  synthetic oligonucleotide
ccgtcgtggccggccacg

SEQ ID NO: 182 - DNA, 18 bp, Artificial Sequence
  synthetic oligonucleotide
  misc_feature (7)..(7): n = U
cgtggcnggccacgacgg

SEQ ID NO: 183 - DNA, 18 bp, Artificial Sequence
  synthetic oligonucleotide
ccgtcgtggccagccacg

SEQ ID NO: 184 - DNA, 18 bp, Artificial Sequence
  synthetic oligonucleotide
cgtggctggccacgacgg

SEQ ID NO: 185 - DNA, 18 bp, Artificial Sequence
  synthetic oligonucleotide
ccgtcgtggccagccacg

SEQ ID NO: 186 - protein, 258 aa, Artificial Sequence
  recombinant peptide
MGHHHHHHSKKSGKSAKSKEKQEKITDTFKVKRKVDRLDDATNKKRKVFVSTILTFWNTDRRDFPWRHTRDPYVILITEILLRRTTAGHVKKIYDKFFVKYKCFEDILKTPKSEIAKDIKEIGLSNQRAEQLKELARVVINDYGGRVPRNRKAILDLPGVGKKTCAAVMCLAFGKKAAMVDANFVRVINRYFGGSYENLNYNHKALWELAETLVPGGKCRDFNLGLMDFSAIICAPRKPKCEKCGMSKLCSYYEKCST

SEQ ID NO: 187 - protein, 221 aa, Artificial Sequence
  recombinant peptide
LDDATNKKRKVFVSTILTFWNTDRRDFPWRHTRDPYVILITEILLRRTTAGHVKKIYDKFFVKYKCFEDILKTPKSEIAKDIKEIGLSNQRAEQLKELARVVINDYGGRVPRNRKAILDLPGVGKKTCAAVMCLAFGKKAAMVDANFVRVINRYFGGSYENLNYNHKALWELAETLVPGGKCRDFNLGLMDFSAIICAPRKPKCEKCGMSKLCSYYEKCST

SEQ ID NO: 188 - protein, 14 aa, Artificial Sequence
  recombinant peptide
NRKAILDLPGVGKK

SEQ ID NO: 189 - protein, 250 aa, Artificial Sequence
  recombinant peptide
SKKSGKSAKSKEKQEKITDTFKVKRKVDRLDDATNKKRKVFVSTILTFWNTDRRDFPWRHTRDPYVILITEILLRRTTAGHVKKIYDKFFVKYKCFEDILKTPKSEIAKDIKEIGLSNQRAEQLKELARVVINDYGGRVPRNRKAILDLPGVGKKTCAAVMCLAFGKKAAMVDANFVRVINRYFGGSYENLNYNHKALWELAETLVPGGKCRDFNLGLMDFSAIICAPRKPKCEKCGMSKLCSYYEKCST

SEQ ID NO: 190 - DNA, 23 bp, Artificial Sequence
  synthetic oligonucleotide
tgtgggcaaaaaaacctgcgcgg

SEQ ID NO: 191 - DNA, 19 bp, Artificial Sequence
  synthetic oligonucleotide
cccggcagatccagaatcg

SEQ ID NO: 192 - protein, 14 aa, Artificial Sequence
  recombinant peptide
ALWELAETLVPGGK

SEQ ID NO: 193 - protein, 18 aa, Artificial Sequence
  recombinant peptide
VINRYFGGSYENLNYNHK

SEQ ID NO: 194 - protein, 16 aa, Artificial Sequence
  recombinant peptide
ALWELAETLVPGGKCR

SEQ ID NO: 195 - protein, 17 aa, Artificial Sequence
  recombinant peptide
YKCFEDILKTPKSEIAK

SEQ ID NO: 196 - protein, 10 aa, Artificial Sequence
  recombinant peptide
AILDLPGVGK

SEQ ID NO: 197 - protein, 12 aa, Artificial Sequence
  recombinant peptide
YKCFEDILKTPK

SEQ ID NO: 198 - protein, 32 aa, Artificial Sequence
  recombinant peptide
VINRYFGGSYENLNYNHKALWELAETLVPGGK

SEQ ID NO: 199 - protein, 13 aa, Artificial Sequence
  recombinant peptide
GHHHHHHSKKSGK

SEQ ID NO: 200 - protein, 12 aa, Artificial Sequence
  recombinant peptide
TCAAVMCLAFGK

SEQ ID NO: 201 - protein, 9 aa, Artificial Sequence
  recombinant peptide
YKCFEDILK

SEQ ID NO: 202 - protein, 7 aa, Artificial Sequence
  recombinant pepetide
CFEDILK

SEQ ID NO: 203 - protein, 16 aa, Artificial Sequence
  recombinant peptide
VINRYFGGSYENLNYN

SEQ ID NO: 204 - protein, 21 aa, Artificial Sequence
  recombinant peptide
SEIAKDIKEIGLSNQRAEQLK

SEQ ID NO: 205 - protein, 11 aa, Artificial Sequence
  recombinant peptide
KIYDKFFVKYK

SEQ ID NO: 206 - protein, 12 aa, Artificial Sequence
  recombinant peptide
KAILDLPGVGKK

SEQ ID NO: 207 - protein, 17 aa, Artificial Sequence
  recombinant peptide
EIGLSNQRAEQLKELAR

SEQ ID NO: 208 - protein, 11 aa, Artificial Sequence
  recombinant peptide
AILDLPGVGKK

SEQ ID NO: 209 - protein, 8 aa, Artificial Sequence
  recombinant peptide
CEKCGMSK

SEQ ID NO: 210 - protein, 10 aa, Artificial Sequence
  recombinant peptide
GHHHHHHSKK

SEQ ID NO: 211 - protein, 10 aa, Artificial Sequence
  recombinant peptide
ITDTFKVKRK

SEQ ID NO: 212 - protein, 20 aa, Artificial Sequence
  recombinant peptide
DIKEIGLSNQRAEQLKELAR

SEQ ID NO: 213 - protein, 12 aa, Artificial Sequence
  recombinant peptide
RKVDRLDDATNK

SEQ ID NO: 214 - protein, 9 aa, Artificial Sequence
  recombinant peptide
GHHHHHHSK

SEQ ID NO: 215 - protein, 10 aa, Artificial Sequence
  recombinant peptide
LCSYYEKCST

SEQ ID NO: 216 - protein, 7 aa, Artificial Sequence
  recombinant peptide
YDKFFVK

SEQ ID NO: 217 - protein, 10 aa, Artificial Sequence
  recombinant peptide
LDDATNKKRK

SEQ ID NO: 218 - protein, 12 aa, Artificial Sequence
  recombinant peptide
VDRLDDATNKKR

SEQ ID NO: 219 - protein, 13 aa, Artificial Sequence
  recombinant peptide
RTTAGHVKKIYDK

SEQ ID NO: 220 - protein, 14 aa, Artificial Sequence
  recombinant peptide
VINRYFGGSYENLN

SEQ ID NO: 221 - protein, 13 aa, Artificial Sequence
  recombinant peptide
TCAAVMCLAFGKK

SEQ ID NO: 222 - protein, 6 aa, Artificial Sequence
  recombinant peptide
KPKCEK

SEQ ID NO: 223 - protein, 5 aa, Artificial Sequence
  recombinant peptide
VVIND

SEQ ID NO: 224 - protein, 12 aa, Artificial Sequence
  recombinant peptide
VINRYFGGSYEN

SEQ ID NO: 225 - protein, 12 aa, Artificial Sequence
  recombinant peptide
YFGGSYENLNYN

SEQ ID NO: 226 - protein, 9 aa, Artificial Sequence
  recombinant peptide
LDLPGVGKK

SEQ ID NO: 227 - protein, 8 aa, Artificial Sequence
  recombinant peptide
RTTAGHVK

SEQ ID NO: 228 - protein, 7 aa, Artificial Sequence
  recombinant peptide
ITSILLR

SEQ ID NO: 229 - protein, 28 aa, Artificial Sequence
  recombinant peptide
AAMVDANFVRVINRYFGGSYENLNYNHK

SEQ ID NO: 230 - protein, 8 aa, Artificial Sequence
  recombinant peptide
SGKSAKSK

SEQ ID NO: 231 - protein, 8 aa, Artificial Sequence
  recombinant peptide
VFVSTILT

SEQ ID NO: 232 - protein, 19 aa, Artificial Sequence
  recombinant peptide
NYNHKALWELAETLVPGGK

SEQ ID NO: 233 - protein, 12 aa, Artificial Sequence
  recombinant peptide
CGMSKLCSYYEK

SEQ ID NO: 234 - protein, 19 aa, Artificial Sequence
  recombinant peptide
VMCLAFGKKAAMVDANFVR

SEQ ID NO: 235 - protein, 6 aa, Artificial Sequence
  recombinant peptide
DKFFVK

SEQ ID NO: 236 - protein, 5 aa, Artificial Sequence
  recombinant peptide
SYYEK

SEQ ID NO: 237 - protein, 12 aa, Artificial Sequence
  recombinant peptide
CAAVMCLAFGKK

SEQ ID NO: 238 - protein, 8 aa, Artificial Sequence
  recombinant peptide
DDATNKKR

SEQ ID NO: 239 - protein, 8 aa, Artificial Sequence
  recombinant peptide
DLPGVGKK

SEQ ID NO: 240 - protein, 13 aa, Artificial Sequence
  recombinant peptide
KAILDLPGVGKKT

SEQ ID NO: 241 - protein, 7 aa, Artificial Sequence
  recombinant peptide
TDTFKVK

SEQ ID NO: 242 - protein, 10 aa, Artificial Sequence
  recombinant peptide
ILDLPGVGKK

SEQ ID NO: 243 - protein, 12 aa, Artificial Sequence
  recombinant peptide
GGSYENLNYNHK

SEQ ID NO: 244 - protein, 8 aa, Artificial Sequence
  recombinant peptide
DFPWRHTR

SEQ ID NO: 245 - protein, 11 aa, Artificial Sequence
  recombinant peptide
CAAVMCLAFGK

SEQ ID NO: 246 - protein, 8 aa, Artificial Sequence
  recombinant peptide
RLDDATNK

SEQ ID NO: 247 - protein, 7 aa, Artificial Sequence
  recombinant peptide
QLKELAR

SEQ ID NO: 248 - protein, 5 aa, Artificial Sequence
  recombinant peptide
LSNQR

SEQ ID NO: 249 - protein, 5 aa, Artificial Sequence
  recombinant peptide
SAKSK

SEQ ID NO: 250 - protein, 6 aa, Artificial Sequence
  recombinant peptide
SKKSGK

SEQ ID NO: 251 - protein, 11 aa, Artificial Sequence
  recombinant peptide
HTRDPYVILIT

SEQ ID NO: 252 - protein, 5 aa, Artificial Sequence
  recombinant peptide
AGHVK

SEQ ID NO: 253 - protein, 9 aa, Artificial Sequence
  recombinant peptide
SKLCSYYEK

SEQ ID NO: 254 - protein, 6 aa, Artificial Sequence
  recombinant peptide
LKELAR

SEQ ID NO: 255 - protein, 5 aa, Artificial Sequence
  recombinant peptide
GHVKK